(19) United States
(12) Patent Application Publication
(10) Pub. No.: US 2006/0246543 A1
(43) Pub. Date: Nov. 2, 2006

(54) Slim compositions and methods of use thereof

(75) Inventors: Michael J. Grusby, Newton, MA (US); Takashi Tanaka, Kanagawa (JP)

Correspondence Address: LAHIVE & COCKFIELD, 28 STATE STREET, BOSTON, MA, 02109, US

(73) Assignee: President and Fellows of Harvard College, Cambridge, MA (US)

(21) Appl. No.: 11/368,166

(22) Filed: Mar. 3, 2006

Related U.S. Application Data
(60) Provisional application No. 60/658,532, filed on Mar. 3, 2005.

(51) Int. Cl. A61K 38/17; C07K 14/47; C07H 21/04; C12P 21/06

(52) U.S. Cl. 435/69.1; 435/320.1; 435/325; 514/12; 530/350; 536/23.5

(57) ABSTRACT

This invention is based, at least in part, on the discovery of a novel nuclear protein which contains both PDZ and LIM domains, SLIM (STAT-interacting LIM). SLIM interacts with activated STAT molecules. The invention also provides methods of using these novel SLIM compositions. The invention also provides therapeutic methods involving the SLIM nucleic acid and protein molecules of the invention.

### RELATED APPLICATIONS

This application claims the benefit of and priority to U.S. Provisional Patent Application Ser. No. 60/658,532, filed Mar. 3, 2005, the entire contents of which is incorporated herein by reference.

### GOVERNMENT FUNDING

Work described herein was supported, at least in part, by National Institutes of Health (NIH) under grants GM-062135 and AI-506296. The government may therefore have certain rights in this invention.

### BACKGROUND OF THE INVENTION

Signal transducers and activators of transcription (STAT) proteins are a family of latent cytoplasmic transcription factors that are activated by tyrosine phosphorylation in response to a variety of cytokines, growth factors and hormones (reviewed in Levy, D. E. & Darnell, J. E. J. (2002) Nat Rev Mol Cell Biol 3, 651-662). Once activated, STAT proteins translocate into the nucleus and help coordinate gene transcription. One striking feature of STAT signaling is its rapid and transient activation and deactivation cycle (Haspel, R. L. & Darnell, J. E. J. (1999) Proc Natl Acad Sci U S A 96, 10188-10193), although the molecular mechanisms responsible for this remain poorly understood.

Several mechanisms for the regulation of STAT signaling have been proposed (Shuai, K. & Liu, B. (2003) Nat Rev Immunol 3, 900-911). For example, numerous tyrosine phosphatases have been reported to act at different levels in the signaling cascade. In addition, the suppressor of cytokine signaling (SOCS) and protein inhibitor of STAT (PIAS) families of proteins have been shown to bind to and inhibit either the cytokine receptor-associated Janus kinase (JAK) or activated STAT molecule, respectively. Other posttranslational modifications of STAT proteins, such as arginine methylation (Mowen, K. A., et al. (2001) Cell 104, 731-741) and ubiquitination (Kim, T. K. & Maniatis, T. (1996) Science 273, 1717-1719; Wang, K. S., Zorn, E. & Ritz, J. (2001) Blood 97, 3860-3866) have also been suggested as important means to regulate STAT signaling, although these mechanisms remain poorly defined.

STAT4 is one of seven mammalian STAT family members and is activated following stimulation by IL-12 or IFN-α (Nguyen, K. B. et al. (2002) Science 297, 2063-2066). STAT4 is essential for IL-12-mediated differentiation of naïve Th cells into IFNγ-secreting Th1 cells as evidenced by the phenotype of STAT4-deficient mice (Kaplan, M. H., et al. (1996) Nature 382, 174-177; Thierfelder, W. E. et al. (1996) Nature 382, 171-174). An understanding of the mechanism by which STAT4 signaling is regulated and methods for modulating STAT4-mediated signaling are lacking in the art.

### SUMMARY OF THE INVENTION

This invention is based, at least in part, on the discovery of a novel nuclear protein which contains both PDZ and LIM domains, SLIM (STAT-interacting LIM). SLIM interacts with activated STAT molecules. The invention also provides methods of using these novel SLIM compositions. The invention also provides therapeutic methods involving the SLIM nucleic acid and protein molecules of the invention.

In one aspect the invention provides an isolated nucleic acid molecule, comprising the coding sequence of the nucleotide sequence set forth in SEQ ID NO.:1, or a complement thereof. In one embodiment, the nucleic acid molecule is RNA. In another embodiment, the nucleic acid molecule is hybridized to a complementary nucleic acid molecule to form a double-stranded molecule.

Another aspect of the invention provides an isolated nucleic acid molecule comprising the nucleotide sequence set forth in SEQ ID NO.:1, or a complement thereof.

Yet another aspect of the invention provides an isolated nucleic acid molecule which has at least 95% identity to the nucleotide sequence set forth in SEQ ID NO.:1 over its full length and which encodes a polypeptide that binds to a STAT molecule. In one embodiment, the STAT is STAT4. In another embodiment, the STAT is STAT1.

One aspect of the invention provides an isolated nucleic acid molecule which has at least 95% identity to the nucleotide sequence set forth in SEQ ID NO.:1 over its full length and which encodes a polypeptide that modulates an activity selected from the group consisting of: STAT ubiquitination, STAT phosphorylation, IFN-γ production, STAT signaling, and Th1 cell differentiation. In one embodiment, the STAT is STAT4. In another embodiment, the STAT is STAT I.

Another aspect of the invention provides an isolated nucleic acid molecule comprising a nucleotide sequence encoding a polypeptide comprising the amino acid sequence set forth in SEQ ID NO.:2.

Another aspect of the invention provides an isolated nucleic acid molecule comprising the coding sequence of SEQ ID NO:1 and a nucleotide sequence encoding a non-SLIM polypeptide.

Yet another aspect of the invention provides an isolated nucleic acid molecule which is complementary to the nucleic acid molecule of any one of the isolated nucleic acid molecules of the invention. Another aspect of the invention provides a vector comprising the nucleic acid molecule of any one of the isolated nucleic acid molecules of the invention. In one embodiment, the vector is an expression vector. Yet another aspect of the invention provides a host cell containing the vectors of the invention.

One aspect of the invention provides a method for producing a polypeptide that binds to STAT, comprising culturing the host cells of the invention in a suitable medium until the polypeptide is produced. In one embodiment, the STAT is STAT4. In another embodiment, the STAT is STAT1. In a further embodiment, the method comprises isolating the polypeptide from the medium or the host cell.

One aspect of the invention provides an isolated polypeptide produced using the methods of the invention.

Another aspect of the invention provides an isolated polypeptide, comprising the amino acid sequence encoded by a nucleic acid molecule comprising the coding region of SEQ ID NO:1.

Yet another aspect of the invention provides an isolated polypeptide comprising the amino acid sequence set forth in SEQ ID NO.:2.

One aspect of the invention provides an isolated protein consisting of the amino acid sequence of SEQ ID NO.:2.

Another aspect of the invention provides an isolated polypeptide comprising an amino acid sequence which has at least 95% amino acid identity to the polypeptide set forth in SEQ ID NO:2 and binds to a STAT molecule. In one embodiment, the STAT is STAT4. In another embodiment, the STAT is STAT1. In another embodiment, the polypeptide has at least 95% amino acid identity across the full length of the polypeptide set forth in SEQ ID NO:2 and modulates an activity selected from the group consisting of: STAT ubiquitination, STAT phosphorylation, IFN-γ production, STAT signaling, and Th1 cell differentiation.

One aspect of the invention provides a fusion protein comprising the amino acid sequence of SEQ ID NO:2 operatively linked to a non-SLIM polypeptide.

Another aspect of the invention provides an antibody that specifically binds to a polypeptide encoded by the amino acid sequence set forth in SEQ ID NO.:2. In one embodiment, the antibody is a polyclonal or monoclonal antibody. In another embodiment, the antibody is a fully human antibody. In a further embodiment, the antibody is a humanized or chimeric antibody. In yet another embodiment, the antibody is an intracellular antibody. In one embodiment, the antibody is coupled to a detectable label.

One aspect of the invention provides a transgenic mouse comprising in its genome an exogenous DNA molecule that functionally disrupts a nucleic acid molecule encoding a polypeptide comprising the amino acid sequence of SEQ ID NO.:2 in said mouse, wherein said mouse exhibits a phenotype characterized by increased IFN-γ production and increased phosphorylation of STAT4 relative to a wild-type mouse.

Another aspect of the invention provides an isolated cell from the transgenic mouse. In one embodiment, the cell is selected from the group consisting of fertilized egg cells, embryonic stem cells and lymphoid cells.

One aspect of the invention provides a method for identifying a compound that modulates the activity of a polypeptide comprising the consensus amino acid sequences shown in SEQ ID NO.:3, comprising providing an indicator composition that comprises a nucleic acid molecule encoding the polypeptide operatively linked to a nucleotide sequence controlling its expression and a target molecule; contacting the indicator composition with a library of test compounds; determining the effect of the test compound on the expression and/or activity of the polypeptide in the indicator composition; and selecting from the library of test compounds a compound of interest that modulates the expression and/or activity of the polypeptide; to thereby identify a compound that modulates the activity of the polypeptide comprising the consensus amino acid sequences shown in SEQ ID NO.:3. In one embodiment, the activity of the polypeptide is E3 ligase ubiquitin activity.

Another aspect of the invention provides a method for identifying a compound which inhibits the E3 ubiquitin ligase activity of a polypeptide comprising the consensus amino acid sequence shown in SEQ ID NO.:3 comprising contacting in the presence of the compound, the polypeptide and a target molecule under conditions which allow ubiquitination of the target molecule by the polypeptide; and detecting the target molecule in which the ability of the compound to inhibit the ubiquitination of the target molecule by the polypeptide is indicated by a decrease in ubiquitination of the target molecule as compared to the amount of ubiquitination of the target molecule in the absence of the compound. In one embodiment, the polypeptide further comprises a consensus PDZ domain comprising the amino acid sequence shown SEQ ID NO:24.

Yet another aspect of the invention provides a method for identifying a compound which inhibits the interaction of a polypeptide comprising the consensus amino acid sequence shown in SEQ ID NO.:3 with a STAT molecule comprising contacting in the presence of the compound, the polypeptide and the STAT molecule under conditions which allow binding of the STAT molecule to the polypeptide to form a complex; and detecting the formation of a complex of the polypeptide and the STAT molecule in which the ability of the compound to inhibit interaction between the polypeptide and the STAT molecule is indicated by a decrease in complex formation as compared to the amount of complex formed in the absence of the compound.

One aspect of the invention provides a method for identifying a compound that modulates the activity of a STAT molecule, comprising providing an indicator composition that comprises a STAT molecule and a polypeptide comprising the consensus amino acid sequence shown in SEQ ID NO.:3 operatively linked to a nucleotide sequence controlling its expression; contacting the indicator composition with a library of test compounds; determining the effect of the test compound on the expression and/or activity of the polypeptide in the indicator composition; and selecting from the library of test compounds a compound of interest that modulates the expression and/or activity of the polypeptide; to thereby identify a compound that modulates the activity of a STAT molecule. In one embodiment, the STAT is STAT4. In another embodiment, the STAT is STAT1.

In one embodiment of the methods of the invention, the indicator composition comprises a polypeptide comprising an amino acid sequence selected from the group consisting of: SEQ ID NOs.:2, 5, 7, 9, 11, 13, 15, and 17, and the effect of the test compound on the activity of the polypeptide is determined in the presence and absence of the test compound. In yet another embodiment, the polypeptide comprises a consensus PDZ domain comprising an amino acid sequence shown SEQ ID NO:24. In one embodiment, the polypeptide comprises a LIM domain shown in SEQ ID NO.:20. In another embodiment, the polypeptide comprises the amino acid sequence shown in SEQ ED NO:2. In yet another embodiment, the polypeptide comprises an amino acid sequence selected from the group consisting of SEQ ID NOs:9, 11, and 13. In another embodiment, the activity is selected from the group consisting of: modulation of the STAT phosphorylation, modulation of IFN-γ production, modulation of STAT ubiquitination, modulation of STAT signaling, and modulation of Th1 cell differentiation. In one embodiment, the modulation of STAT phosphorylation, modulation of STAT ubiquitination, and modulation of STAT signaling, is modulation of STAT4 phosphorylation, modulation of STAT4 ubiquitination, and modulation of STAT4 signaling. In another embodiment, the modulation of STAT phosphorylation, modulation of STAT ubiquitination, and modulation of STAT signaling, is modulation of STAT1 phosphorylation, modulation of STAT1 ubiquitination, and modulation of STAT1 signaling. In one embodiment, the indicator composition is a cell free composition. In another embodiment, the indicator composition is a cell based composition. In one embodiment, the cell is selected from the group consisting of: a T cell, a B cell, and a macrophage. In yet another embodiment, the cell is a Th1 cell. In one embodiment, the methods of the invention further comprise determining the effect of the test compound on an immune response in a subject.

Another aspect of the invention provides a method of modulating IFN-γ production by a cell comprising contacting the cell with an agent that downmodulates the expression and/or activity of a polypeptide comprising an amino acid sequence set forth in any one of SEQ ID NOs.:2, 5, 7, 9, 11, 13, 15, and 17, wherein the agent is selected from the group consisting of: an intracellular antibody that binds to the polypeptide, a nucleic acid molecule that mediates RNAi, a nucleic acid molecule that is antisense to an amino acid sequence set forth in SEQ ID NOs.:2, 5, 7, 9, 11, 13, 15, and 17, a dominant negative of an amino acid sequence set forth in SEQ ID NOs.:2, 5, 7, 9, 11, 13, 15, and 17, and a small molecule antagonist of an amino acid sequence set forth in SEQ ID NOs.:2, 5, 7, 9, 11, 13, 15, and 17, such that IFN-γ production is modulated

Yet another aspect of the invention provides a method of modulating IFN-γ production by a cell comprising contacting the cell with an agent that upmodulates the expression and/or activity of a polypeptide comprising an amino acid sequence set forth in any one of SEQ ID NOs.:2, 5, 7, 9, 11, 13, 15, and 17, wherein the agent is selected from the group consisting of: a nucleic acid molecule encoding a polypeptide comprising an amino acid sequence set forth in SEQ ID NOs.:2, 5, 7, 9, 11, 13, 15, and 17, a polypeptide comprising an amino acid sequence set forth in SEQ ID NOs.: 2, 5, 7, 9, 11, 13, 15, and 17, a small molecule agonist of an amino acid sequence set forth in SEQ ID NOs.:2, 5, 7, 9, 11, 13, 15, and 17, such that IFN-γ production by the cell is modulated. In one embodiment, the cell is a T cell. In another embodiment, the cell is a Th1 cell.

One aspect of the invention provides a method of treating or preventing a disorder that would benefit from treatment with an agent that modulates the activity of a STAT polypeptide, comprising administering to a subject with said disorder an agent that downmodulates the expression and/or activity of a polypeptide comprising an amino acid sequence set forth in any one of SEQ ID NOs.: 2, 5, 7, 9, 11, 13, 15, and 17, wherein the agent is selected from the group consisting of: an intracellular antibody that binds to the polypeptide, a nucleic acid molecule that mediates RNAi, a nucleic acid molecule that is antisense to an amino acid sequence set forth in SEQ ID NOs.: 2, 5, 7, 9, 11, 13, 15, and 17, a dominant negative of an amino acid sequence set forth in SEQ ID NOs.: 2, 5, 7, 9, 11, 13, 15, and 17, a small molecule agonist of an amino acid sequence set forth in SEQ ID NOs.: 2, 5, 7, 9, 11, 13, 15, and 17, and a small molecule antagonist of an amino acid sequence set forth in SEQ ID NOs.: 2, 5, 7, 9, 11, 13, 15, and 17, such that the disorder is treated or prevented. In one embodiment, the STAT polypeptide is STAT4. In another embodiment, the STAT polypeptide is STAT1.

Yet another aspect of the invention provides a method of treating or preventing a disorder that would benefit from treatment with an agent that modulates the activity of a STAT polypeptide, comprising administering to a subject with said disorder an agent that upmodulates the expression and/or activity of a polypeptide comprising an amino acid sequence set forth in any one of SEQ ID NOs.: 2, 5, 7, 9, 11, 13, 15, and 17, wherein the agent is selected from the group consisting of: a nucleic acid molecule encoding a polypeptide comprising an amino acid sequence set forth in SEQ ID NOs.: 2, 5, 7, 9, 11, 13, 15, and 17, a polypeptide comprising an amino acid sequence set forth in SEQ ID NOs.: 2, 5, 7, 9, 11, 13, 15, and 17, a small molecule agonist of an amino acid sequence set forth in SEQ ID NOs.: 2, 5, 7, 9, 11, 13, 15, and 17, such that such that the disorder is treated or prevented.

One aspect of the invention provides a method of modulating protein folding, protein transport and/or protein secretion by a cell comprising contacting the cell with an agent that downmodulates the expression and/or activity of a polypeptide comprising an amino acid sequence set forth in any one of SEQ ID NOs.: 2, 5, 7, 9, 11, 13, 15, and 17, wherein the agent is selected from the group consisting of: an intracellular antibody that binds to the polypeptide, a nucleic acid molecule that mediates RNAi, a nucleic acid molecule that is antisense to an amino acid sequence set forth in SEQ ID NOs.: 2, 5, 7, 9, 11, 13, 15, and 17, a dominant negative of an amino acid sequence set forth in SEQ ID NOs.: 2, 5, 7, 9, 11, 13, 15, and 17, and a small molecule antagonist of an amino acid sequence set forth in SEQ ID NOs.: 2, 5, 7, 9, 11, 13, 15, and 17, such that protein folding, protein transport and/or protein secretion is modulated

Another aspect of the invention provides a method of modulating protein folding, protein transport and/or protein secretion by a cell comprising contacting the cell with an agent that upmodulates the expression and/or activity of a polypeptide comprising an amino acid sequence set forth in any one of SEQ ID NOs.: 2, 5, 7, 9, 11, 13, 15, and 17, wherein the agent is selected from the group consisting of: a nucleic acid molecule encoding a polypeptide comprising an amino acid sequence set forth in SEQ ID NOs.: 2, 5, 7, 9, 11, 13, 15, and 17, a polypeptide comprising an amino acid sequence set forth in SEQ ID NOs.: 2, 5, 7, 9, 11, 13, 15, and 17, a small molecule agonist of an amino acid sequence set forth in SEQ ID NOs.: 2, 5, 7, 9, 11, 13, 15, and 17, such that protein folding, protein transport and/or protein secretion by the cell is modulated.

Yet another aspect of the invention provides a method of modulating protein folding, protein transport and/or protein secretion by a cell comprising contacting the cell with an agent that downmodulates the expression and/or activity of a polypeptide comprising an amino acid sequence set forth in any one of SEQ ID NOs.: 2, 5, 7, 9, 11, 13, 15, and 17, wherein the agent is selected from the group consisting of: an intracellular antibody that binds to the polypeptide, a nucleic acid molecule that mediates RNAi, a nucleic acid molecule that is antisense to an amino acid sequence set forth in SEQ ID NOs.: 2, 5, 7, 9, 11, 13, 15, and 17, a dominant negative of an amino acid sequence set forth in SEQ ID NOs.: 2, 5, 7, 9, 11, 13, 15, and 17, and a small molecule antagonist of an amino acid sequence set forth in SEQ ID NOs.: 2, 5, 7, 9, 11, 13, 15, and 17, such that protein degradation is modulated

Another aspect of the invention provides a method of modulating protein degradation by a cell comprising contacting the cell with an agent that upmodulates the expression and/or activity of a polypeptide comprising an amino acid sequence set forth in any one of SEQ ID NOs.: 2, 5, 7, 9, 11, 13, 15, and 17, wherein the agent is selected from the group consisting of: a nucleic acid molecule encoding a polypeptide comprising an amino acid sequence set forth in SEQ ID NOs.: 2, 5, 7, 9, 11, 13, 15, and 17, a polypeptide comprising an amino acid sequence set forth in SEQ ID NOs.: 2, 5, 7, 9, 11, 13, 15, and 17, a small molecule agonist of an amino acid sequence set forth in SEQ ID NOs.: 2, 5, 7, 9, 11, 13, 15, and 17, such that protein degradation by the cell is modulated.

### BRIEF DESCRIPTION OF THE DRAWINGS

FIGS. 1A-D show that SLIM is a nuclear PDZ-LIM protein which can interact with activated STAT proteins. (A) Top: schematic diagram of SLIM structure. Bottom: predicted amino acid sequence of mouse SLIM. PDZ and LIM domains are boxed and conserved cysteine and histidine residues in the LIM domain are underlined. (B) Northern blot analysis of SLIM expression in mouse tissues (left) and primary cells (right). Full-length SLIM cDNA was used as a probe, and probes for HPRT or β-actin were used as controls. (C) Western blot analysis of SLIM. Cytoplasmic (C) and nuclear (N) extracts from CD4+ T cells, untreated or treated with IL-12 (10 ng/ml) for 30 min, were subjected to immunoblot (IB) with SLIM antisera. (D) SLIM can interact with activated STAT4. 293T cells were transfected with expression plasmids for His-SLIM (WT) or a frame shift (FS) mutant along with STAT4 or STAT4 (Y693). Nuclear extracts, untreated or treated with human IFNα (1000 U/ml) for 30 min, were immunoprecipitated (IP) with anti-His and immunoblotted (IB) with anti-STAT4.

FIGS. 2A-B show that SLIM negatively regulates STAT4-mediated signaling. (A) SLIM inhibits STAT4-mediated transactivation. U3A cells were transfected with a (2×)IRF-1 luciferase reporter construct and expression plasmids for STAT4, with or without SLIM. Luciferase activity was measured with (filled bars) or without (open bars) stimulation with human IFNα(1000 U/ml) for 5 h (left). U3A cells, which were stably transfected with IL-12 receptor β1 and β2 chain expression plasmids, were transfected as indicated and stimulated with human IL-12 (10 ng/ml) for 5 h before luciferase activity was measured (right). (B) SLIM inhibits STAT4-mediated IFN-γ production in response to IL-12 in Th1 cell lines. 2D6 cell clones, stably transfected with empty vector (C1, C2) or SLIM (S1, S2), were stimulated with IL-12 (12.5 ng/ml) for 72 h, at which time IFN-γ production was measured by ELISA.

FIGS. 3A-F show that SLIM is an E3 ligase which can promote the ubiquitination and degradation of STAT proteins. (A) In vitro autoubiquitination assay for SLIM. Recombinant SLIM was incubated in vitro with ubiquitin components as indicated. Ubiquitinated SLIM was detected by immunoblot with avidin-HRP. (B) SLIM promotes ubiquitination of STAT4 in vivo. 293T cells were transfected with expression plasmids for His-ubiquitin, STAT4 and SLIM (WT) or frame shift (FS) mutant, and treated with MG132 (20 nM) for 1 h followed by stimulation with IFNα (1000 U/ml) for 1 h. His-tagged proteins were purified using Ni-NTA beads and immunoblotted with anti-STAT4. (C) SLIM promotes ubiquitination of STAT4 in vitro. STAT4 proteins, immunoprecipitated with anti-STAT4 from ConA-activated thymocytes, were incubated in vitro with ubiquitin components as indicated in the absence or presence of recombinant SLIM. Ubiquitinated STAT4 was detected by immunoblot with anti-STAT4. (D) SLIM decreases the steady state level of STAT4 protein. 293T cells were transfected with a fixed amount of STAT4 and increasing amounts of SLIM expression plasmids. Whole cell extracts were subjected to immunoblot with the indicated antibodies. (E) STAT4 degradation by SLIM is dependent on 26S proteosome activity. 293T cells were transfected with expression plasmids for Flag-STAT4 and SLIM or frame shift mutant. Transfected cells were incubated in the absence or presence of MG132 (20 nM) for 6 h and then stimulated with IFNα (1000 U/ml) for 1 h. Whole cell extracts were prepared, immunoprecipitated with anti-Flag and immunoblotted with anti-STAT4. (F) SLIM neither ubiquitinates nor degrades p53. 293T cells were transfected with expression plasmids for His-ubiquitin, Flag-p53 and SLIM or MDM2. Whole cell extracts were immunoprecipitated and immunoblotted with anti-Flag (left). SLIM or MDM2 were transfected with Flag-p53 into 293T cells and steady state levels of p53 protein were assessed by immunoblotting with anti-Flag (right).

FIGS. 4A-B show that Th1 cell differentiation is enhanced and Stat4 protein levels are increased in SLIM-deficient CD4+ T cells. (A) CD4+ T cells were purified from lymph nodes of wild type (+/+) or SLIM-deficient (−/−) mice and stimulated in vitro with anti-CD3 and anti-CD2S in the presence of IL-12. IFNγ production upon primary (left) and secondary (middle) stimulation was assessed by ELISA. Total spleen cells were cultured in the presence of heat-killed Listeria monocytogenes for 4 days, restimulated with anti-CD3 for 24 h and IFNγ production measured by ELISA (right). (B) CD4+ T cells were purified from spleens of wild type or SLIM-deficient mice. Whole cell extracts were prepared and immunoblotted with anti-STAT4 or HSP90.

### DETAILED DESCRIPTION OF THE INVENTION

This invention is based, at least in part, on the discovery of a novel nuclear protein which contains both PDZ and LIM domains and that interacts with activated STAT4 molecules. In particular, this invention provides isolated nucleic acid molecules encoding SLIM (STAT-interacting LIM) and isolated SLIM proteins. SLIM is an ubiquitin E3 ligase that inhibits the tyrosine and serine phosphorylation of STAT, e.g., STAT4 and/or STAT1, leading to the proteosome-mediated degradation of STAT proteins. Overexpression of SLIM leads to impaired STAT activity, e.g., STAT4 and/or STAT1, due to reduced STAT protein levels, while SLIM deficiency results in increased STAT expression, e.g., STAT4 and/or STAT1, and thus enhanced interferon-γ (IFNγ) production by T helper 1 (Th1) cells. These data are the first to show ubiquitin E3 ligase activity associated with a LIM-domain containing protein and demonstrate that ubiquitination is an important mechanism for negatively regulating the STAT signaling pathway. The invention also provides methods of using these novel SLIM compositions. In particular, the SLIM nucleic acid and protein molecules of the present invention are useful as modulating agents in regulating a variety of cellular processes, e.g., cytokine responses, IFNγ production, and Th1 differentiation. The invention also provides therapeutic methods involving the SLIM nucleic acid and protein molecules of the invention.

The methods of the present invention are not limited to the use of the molecules set forth in SEQ ID NO:1 and SEQ ID NO:2, i.e., murine SLIM, but include structurally related members of the SLIM family, such as for example, rat and human SLIM, or isoforms thereof, which have a SLIM activity, e.g., STAT ubiquitination, STAT phosphorylation, IFN-γ production, STAT signaling, and Th1 cell differentiation.

Accordingly, the “STAT-interacting LIM molecules” or “SLIM molecules” include nucleic acid molecule sharing sequence homology to the nucleic acid molecules shown in SEQ ID NO:1 and SLIM proteins that share amino acid identity with or share distinguishing SLIM structural features, e.g., LIM and/or PDZ domains, of the SLIM proteins shown in SEQ ID NO:2, combined with SLIM function, i.e., those nucleic acid molecules which encode polypeptides or polypeptides having SLIM biological activity. Further structural and functional features of SLIM proteins are provided below. The nucleotide and amino acid sequences of rat SLIM are known and can be found in gi:50925674 (SEQ ID NO:4 and SEQ ID NO:5, respectively), and gi:56090294 (SEQ ID NO:6 and SEQ ID NO:7, respectively); the nucleotide and amino acid sequences of human SLIM are known and can be found in gi:40288188 (SEQ ID NO:8 and SEQ ID NO:9, respectively), gi:18204288 (SEQ ID NO:10 and SEQ ID NO:11, respectively), and gi:47940542 (SEQ ID NO:12 and SEQ ID NO:13, respectively); and additional murine SLIM family members can be found in gi:22122422 (SEQ ID NO:14 and SEQ ID NO:15, respectively) and gi:19354024 (SEQ ID NO:16 and SEQ ID NO:17, respectively).

The term “family” when referring to the polypeptide and nucleic acid molecules of the invention is intended to mean two or more polypeptides or nucleic acid molecules having a common structural domain or motif and having sufficient amino acid or nucleotide sequence homology as defined herein. Such family members can be naturally or non-naturally occurring and can be from either the same or different species. For example, a family can contain a first polypeptide of human origin, as well as at least one other, distinct polypeptide of human origin. Alternatively, a family can contain, e.g., a human polypeptide and at least one ortholog of non-human origin, e.g., a mouse or a monkey polypeptide. Members of a family of polypeptides share common functional characteristics.

As used interchangeably herein, a “SLIM activity,” “biological activity of SLIM,” or “functional activity of SLIM”, refers to an activity exerted by a SLIM protein, polypeptide or nucleic acid molecule on a SLIM responsive cell or tissue, or on a SLIM protein substrate, as determined in vivo, or in vitro, according to standard techniques and methods described herein. In one embodiment, a SLIM activity is a direct activity, such as an association with a SLIM-target molecule. As used herein, a “target molecule” or “binding partner” is a molecule with which a SLIM protein binds or interacts in nature, such that SLIM-mediated function is achieved. An exemplary SLIM target molecule is a STAT molecule, e.g., STAT 1 or STAT4. Alternatively, a SLIM activity is an indirect activity, such as a downstream cellular signaling activity mediated by interaction of the SLIM protein with a SLIM ligand. For example, the SLIM proteins of the present invention can have one or more of the following activities: modulation of STAT ubiquitination, modulation of STAT phosphorylation, modulation of IFN-γ production, modulation of STAT signaling, modulation of Th1 cell differentiation, modulation of protein folding, protein transport, and/or protein secretion, and/or modulation of protein degradation.

Furthermore, based on the discovery that the LIM domain of SLIM (amino acids 282-333 of SEQ ID NO:2 (SEQ ID NO:18)) and/or the PDZ domain of SLIM (amino acids 4-77 of SEQ ID NO:2 (SEQ ID NO:21)) contribute to the biological activity of the SLIM protein, SLIM proteins used in the methods of the invention preferably contain one or both of the following: a LIM domain and/or a PDZ domain.

As used herein, the term “LIM domain” is an art recognized evolutionarily conserved cysteine-histidine rich, zinc-coordinating domain, consisting of two tandemly repeated zinc fingers, that has been identified in a variety of different proteins. Although the LIM domain contains a zinc finger motif, it does not bind to DNA. LIM domain-containing proteins can be either cytoplasmic or nuclear and may contain additional functional motifs. The LIM domain has been shown to mediate protein-protein interactions and has been shown to be involved in a number of biological processes including cell lineage specification, cytoskeletal organization, and organ development. The LIM domain has the consensus amino acid sequence: CX₂ CX₁₆₋₂₃ HX₂ CX₂ CX₂ CX₁₆₋₂₁ CX₂₋₃ (C/H/D) (SEQ ID NO:3) (Retaux, S. and I. Bachy (2002) Mol Neurobiol 26:269; Bach, I. (2000) Mech Develop 91:5, the contents of each of which is incorporated herein by reference). Preferably, a LIM domain comprises a LIM consensus sequence. In one preferred embodiment, a LIM domain comprises the sequence, CKKCSVNISNQAVRIQEGRYPHPGCYTCADCGLNLKMRGHFWVGNELYCEKH (SEQ ID NO:18; amino acids 282-333 of SEQ ID NO:2). In another preferred embodiment, a LIM domain comprises the sequence, CEKCSVNISNQAV RIQEGRYRHPGCYTCADCGLNLKMRGHFWVGNELYCEKH (SEQ ID NO:19; amino acids 283-334 of SEQ ID NO:5 or SEQ ID NO:7). In another preferred embodiment, a LIM domain comprises the sequence, CEKCSVNISNQAV RIQEGRYRHPGCYTCADCGLNLKMRGHFWVGNELYCEKIi (SEQ ID NO:20; amino acids 285-337 of SEQ ID NO:9 or SEQ ID NO:11, or SEQ ID NO:13).

As used herein, a “PDZ domain” (also known a DHR domain or GLGF repeat) is an art recognized motif in a family of proteins that mediate specific protein-protein interactions of approximately 80-90-amino acids in length. The specificity of the PDZ domain is dictated by the primary structure of the PDZ domain as well as its binding target. PDZ domains comprising six beta-strands (betaA to betaF) and two alpha-helices, A and B, compactly arranged in a globular structure containing a “GLGF loop” (Glycine-Leucine-Glycine-Phenylalanine) (see, for example, Cabral, et al. (1996) Nature 382:649). Preferably, a PDZ domain comprises a PDZ consensus sequence (TVXVAGPAPWGFRIXGGRDFHTPIXVTKVXERGKAX₂ADLRPGDIIVAINGXSA EXMLHAEAQSKIRQSXSPLRLQL (SEQ ID NO:24)). In one preferred embodiment, a PDZ domain comprises the sequence, TVDVAGPAPWGFRISGGRDFHTPIIVTKVTERGKAEAADLRPGDIIVAINGQSAE NMLHAEAQSKIRQSASPLRLQL (SEQ ID NO:21; amino acids 4-77 of SEQ ID NO:2). In another preferred embodiment, a PDZ domain comprises the sequence, TVNVVGPAPWGFRISGGRDFHTPIIVTKVTERGKAEAADLRPGDIIVAINGESAES MLHAEAQSKIRQSASPLRLQL (SEQ ID NO:22; amino acids 4-77 of SEQ ID NO:5 or SEQ ID NO:7). In another preferred embodiment, a PDZ domain comprises the sequence, TVDVAGPAPWGFRITGGRDFHTP IMVTKVAERGKAKDADLRPGDIIVAINGESA EGMLHAEAQSKIRQSPSPLRLQL (SEQ ID NO:23; amino acids 4-77 of SEQ ID NO:9 or SEQ ID NO: 11, or SEQ ID NO: 13).

Isolated SLIM polypeptides of the present invention have an amino acid sequence sufficiently identical to the amino acid sequence of SEQ ID NO:2 or are encoded by a nucleotide sequence sufficiently identical to SEQ ID NO: 1. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, polypeptides comprising amino acid sequences having at least about 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identity to SEQ ID NO:2 over its full-length or over the LIM and/or PDZ domain (amino acids 282-333 of SEQ ID NO:2 and/or amino acids 4-77 of SEQ ID NO:2, respectively), are defined herein as sufficiently identical. In a further embodiment, the invention provides an isolated SLIM protein comprising an amino acid sequence at least 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to the amino acid sequences comprising the LIM and/or PDZ domain (amino acids 282-333 of SEQ ID NO:2 and amino acids 4-77 of SEQ ID NO:2, respectively), and having one or more of the amino acid residues specific to the SLIM protein as compared to the human and/or rat SLIM protein. In another embodiment, the invention provides an isolated polypeptide comprising a LIM consensus domain or specific LIM domain sequence described herein that modulates the ubiquitination of STAT. In yet another embodiment, the invention provides an isolated polypeptide comprising a LIM consensus domain that modulates the E3 ubiquitin ligase activity. In another embodiment, the invention provides an isolated polypeptide comprising a PDZ consensus domain that modulates the ubiquitination of STAT. In yet another embodiment, the invention provides an isolated polypeptide comprising a PDZ consensus domain that modulates the E3 ubiquitin ligase activity.

Furthermore, amino acid sequences which are structurally related to SEQ ID NO:2, e.g., either based on sequence homology or the presence of key structural domains, and share a common functional activity, e.g., modulation of STAT ubiquitination, modulation of STAT phosphorylation, modulation of IFN-γ production, modulation of STAT signaling, modulation of Th1 cell differentiation, modulation of protein folding, protein transport, and/or protein secretion, and modulation of protein degradation, are defined herein as sufficiently identical.

The nucleotide and amino acid sequences of the isolated murine SLIM molecule are shown in SEQ ID NOs:1 and 2, respectively.

Certain terms are first defined so that the invention may be more readily understood.

The articles “a” and “an” are used herein to refer to one or to more than one (i.e. to at least one) of the grammatical object of the article. By way of example, “an element” means one element or more than one element.

As used herein, the term “modulated” with respect to SLIM includes changing the expression, activity and/or function of SLIM in such a manner that it differs from the naturally-occurring expression, function and/oror activity of SLIM under the same conditions. For example, the expression, function and/oror activity can be greater or less than that of naturally occurring SLIM, e.g., owing to a change in binding specificity, etc. As used herein, the various forms of the term “modulate” include stimulation (e.g., increasing or upregulating a particular response or activity) and inhibition (e.g., decreasing or downregulating a particular response or activity).

As used herein, the term “compound” includes any agent, e.g., nucleic acid molecules, antisense nucleic acid molecule, peptide, peptidomimetic, small molecule, or other drug, which binds to SLIM proteins or has a stimulatory or inhibitory effect on, for example, SLIM expression or SLIM activity, binding affinity or stability. In one embodiment, the compound may modulate transcription of SLIM.

The term “stimulator” or “stimulatory agent” includes agents, e.g., agonists, which increase the expression and/or activity of SLIM. Exemplary stimulating agents include active protein and nucleic acid molecules, peptides and peptidomimetics of SLIM. Modulatory agents also include naturally occurring modulators, e.g., modulators of expression such as, for example, interferons.

The agents of the invention can directly or indirectly modulate, i.e., increase or decrease, the expression and/or activity of SLIM. Exemplary agents are described herein or can be identified using screening assays that select for such compounds, as described in detail below.

For screening assays of the invention, preferably, the “test compound or agent” screened includes molecules that are not known in the art to modulate SLIM activity and/or expression and/or SLIM biological activity as described herein. Preferably, a plurality of agents are tested using the instant methods.

The term “library of test compounds” is intended to refer to a panel comprising a multiplicity of test compounds.

In one embodiment, the agent or test compound is a compound that directly interacts with SLIM or directly interacts with a molecule with which SLIM interacts (e.g., a compound that inhibits or stimulates the interaction between SLIM and a SLIM target molecule, e.g., DNA or another protein). In another embodiment, the compound is one that indirectly modulates SLIM expression and/or activity, e.g., by modulating the activity of a molecule that is upstream or downstream of SLIM in a signal transduction pathway involving SLIM. Such compounds can be identified using screening assays that select for such compounds, as described in detail below.

As used herein, the term “target molecule” or “binding partner” is a molecule with which SLIM binds or interacts in nature, and which interaction results in a biological response. The target molecule can be a protein or a nucleic acid molecule. Exemplary target molecules of the invention include proteins in the same signaling pathway as the SLIM protein, e.g., proteins which may function upstream (including both stimulators and inhibitors of activity) or downstream of the SLIM protein in a pathway involving for example, modulation of STAT ubiquitination, modulation of STAT phosphorylation, modulation of IFN-γ production, modulation of STAT signaling, modulation of Th1 cell differentiation, modulation of protein folding, protein transport, and/or protein secretion, and/or modulation of protein degradation.

The term “interact” as used herein is meant to include detectable interactions between molecules, such as can be detected using, for example, a yeast two hybrid assay or coimmunoprecipitation. The term interact is also meant to include “binding” interactions between molecules. Interactions may be protein-protein or protein-nucleic acid in nature.

As used herein, the term “contacting” (i.e., contacting a cell e.g. an immune cell, with a compound) is intended to include incubating the compound and the cell together in vitro (e.g., adding the compound to cells in culture) or administering the compound to a subject such that the compound and cells of the subject are contacted in vivo.

As used herein, the term “indicator composition” refers to a composition that includes a protein of interest (e.g., SLIM), for example, a cell that naturally expresses the protein, a cell that has been engineered to express the protein by introducing an expression vector encoding the protein into the cell, or a cell free composition that contains the protein (e.g., purified naturally-occurring protein or recombinantly-engineered protein).

As used herein, the term “cell free composition” refers to an isolated composition which does not contain intact cells. Examples of cell free compositions include cell extracts and compositions containing isolated proteins.

As used herein an “agonist” of the SLIM proteins can retain substantially the same, or a subset, of the biological activities of the naturally occurring form of a SLIM protein. An “antagonist” of a SLIM protein can inhibit one or more of the activities of the naturally occurring form of the SLIM protein by, for example, competitively modulating a cellular activity of a SLIM protein.

As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane, et al. 1998. Science 282:63), and natural product extract libraries. In another embodiment, the compounds are small, organic non-peptidic compounds. In a further embodiment, a small molecule is not biosynthetic.

As used herein, the term “oligonucleotide” includes two or more nucleotides covalently coupled to each other by linkages (e.g., phosphodiester linkages) or substitute linkages.

As used herein, the term “peptide” includes relatively short chains of amino acids linked by peptide bonds. The term “peptidomimetic” includes compounds containing non-peptidic structural elements that are capable of mimicking or antagonizing peptides.

As used herein, the term “reporter gene” includes genes that express a detectable gene product, which may be RNA or protein. Preferred reporter genes are those that are readily detectable. The reporter gene may also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet, et al. (1987), Mol. Cell. Biol. 7:725-737); bacterial luciferase (Engebrecht and Silverman (1984), Proc. Natl. Acad. Sci., USA 1: 4154-4158; Baldwin, et al. (1984), Biochemistry 23: 3663-3667); alkaline phosphatase (Toh, et al. (1989) Eur. J. Biochem. 182: 231-238, Hall, et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO 96/23898).

The term “treatment,” as used herein, is defined as the application or administration of a therapeutic agent to a patient, or application or administration of a therapeutic agent to an isolated tissue or cell line from a patient, who has a disease, disorder, or infection, a symptom of a disease, disorder, or infection or a predisposition toward a disease, disorder, or infection, with the purpose of curing, healing, alleviating, relieving, altering, remedying, ameliorating, improving or affecting the disease, disorder, or infection, the symptoms of disease, disorder, or infection or the predisposition toward a disease, disorder, or infection. A therapeutic agent includes, but is not limited to, nucleic acid molecules, small molecules, peptides, peptidomimetics, antibodies, ribozymes, and sense and antisense oligonucleotides described herein.

As used herein, the term “disorders that would benefit from treatment with an agent that modulates the activity of a STAT polypeptide” includes disorders in which SLIM activity is aberrant or which would benefit from modulation of a SLIM activity. The agent may directly or indirectly increase IFNγ production.

As used herein, the term “signal transducers and activators of transcription” or “STAT” is an art recognized family of unrelated cytoplasmic signaling proteins involved in signal transduction of several cytokines. They function as latent cytoplasmic transcriptional activators that become activated by tyrosine phosphorylation by Janus kinases (JAK proteins) in response to the engagement of various cytokine receptors. Phosphorylated STAT proteins dimerize and subsequently move to the cell nucleus, where they activate transcription by binding to specific DNA elements. Members of the STAT family contain conserved structural features commonly found in transcription factors, e.g., heptad leucine repeats, a helix-turn-helix motif and SH2 and SH3 domains. Different SH2 domains specifically recognize short sequence motifs flanking a tyrosine phosphorylated residue and play a crucial role in signal transduction. SH3 domains are involved in the targeting of signaling components to specific subcellular locations.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurring most frequently in a selected population is often referred to as the reference and/or wild-type form. Other allelic forms are typically designated as alternative or variant alleles. Diploid organisms may be homozygous or heterozygous for allelic forms. A diallelic or biallelic polymorphism has two forms. A trialleleic polymorphism has three forms.

The term “single nucleotide polymorphism” (SNP) refers to a polymorphic site occupied by a single nucleotide, which is the site of variation between allelic sequences. A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNPs may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNPs may also occur in noncoding regions of the nucleotide sequence. This may result in defective protein expression, e.g., as a result of alternative spicing, or it may have no effect.

As used herein, the term “misexpression” includes a non-wild-type pattern of gene expression. Expression as used herein includes transcriptional, post transcriptional, e.g., mRNA stability, translational, and post translational stages. Misexpression includes: expression at non-wild-type levels, i.e., over or under expression; a pattern of expression that differs from wild-type in terms of the time or stage at which the gene is expressed, e.g., increased or decreased expression (as compared with wild-type) at a predetermined developmental period or stage; a pattern of expression that differs from wild-type in terms of decreased expression (as compared with wild-type) in a predetermined cell type or tissue type; a pattern of expression that differs from wild-type in terms of the splicing of the mRNA, amino acid sequence, post-transitional modification, or biological activity of the expressed polypeptide; a pattern of expression that differs from wild-type in terms of the effect of an environmental stimulus or extracellular stimulus on expression of the gene, e.g., a pattern of increased or decreased expression (as compared with wild-type) in the presence of an increase or decrease in the strength of the stimulus. Misexpression includes any expression from a transgenic nucleic acid. Misexpression includes the lack or non-expression of a gene or transgene, e.g., that can be induced by a deletion of all or part of the gene or its control sequences.

As used herein, the term “knockout” refers to an animal or cell therefrom, in which the insertion of a transgene disrupts an endogenous gene in the animal or cell therefrom. This disruption can essentially eliminate, for example, SLIM, in the animal or cell.

In preferred embodiments, misexpression of the gene encoding the SLIM protein is caused by disruption of the SLIM gene. For example, the SLIM gene can be disrupted through removal of DNA encoding all or part of the protein.

As used herein, “disruption of a gene” refers to a change in the gene sequence, e.g., a change in the coding region. Disruption includes: insertions, deletions, point mutations, and rearrangements, e.g., inversions. The disruption can occur in a region of the native SLIM DNA sequence (e.g., one or more exons) and/or the promoter region of the gene so as to decrease or prevent expression of the gene in a cell as compared to the wild-type or naturally occurring sequence of the gene. The “disruption” can be induced by classical random mutation or by site directed methods. Disruptions can be transgenically introduced. The deletion of an entire gene is a disruption. Preferred disruptions reduce SLIM levels to about 50% of wild-type, in heterozygotes or essentially eliminate SLIM in homozygotes.

As used herein, the term “transgenic cell” refers to a cell containing a transgene.

As used herein, a “transgenic animal” refers to a non-human animal, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

As used herein, the term “cells deficient in SLIM” is intended to include cells of a subject that are naturally deficient in SLIM, as wells as cells of a non-human SLIM deficient animal, e.g., a mouse, that have been altered such that they are deficient in SLIM. The term “cells deficient in SLIM” is also intended to include cells isolated from a non-human SLIM deficient animal or a subject that are cultured in vitro.

As used herein, the term “non-human SLIM deficient animal” refers to a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal, such that the endogenous SLIM gene is altered, thereby leading to either no production of SLIM or production of a mutant form of SLIM having deficient SLIM activity. Preferably, the activity of SLIM is entirely blocked, although partial inhibition of SLIM activity in the animal is also encompassed. The term “non-human SLIM deficient animal” is also intended to encompass chimeric animals (e.g., mice) produced using a blastocyst complementation system, such as the RAG-2 blastocyst complementation system, in which a particular organ or organs (e.g., the lymphoid organs) arise from embryonic stem (ES) cells with homozygous mutations of the SLIM gene.

As used herein, the term “T cell” (i.e., T lymphocyte) is intended to include all cells within the T cell lineage, including thymocytes, immature T cells, mature T cells and the like, from a mammal (e.g., human). T cells include mature T cells that express either CD4 or CD8, but not both, and a T cell receptor. The various T cell populations described herein can be defined based on their cytokine profiles and their function.

As used herein “progenitor T cells” (“Thp”) are naïve, pluripotent cells that express CD4.

As used herein, the term “naïve T cells” includes T cells that have not been exposed to cognate antigen and so are not activated or memory cells. Naïve T cells are not cycling and human naïve T cells are CD45RA+. If naïve T cells recognize antigen and receive additional signals depending upon but not limited to the amount of antigen, route of administration and timing of administration, they may proliferate and differentiate into various subsets of T cells, e.g., effector T cells.

As used herein, the term “peripheral T cells” refers to mature, single positive T cells that leave the thymus and enter the peripheral circulation.

As used herein, the term “memory T cell” includes lymphocytes which, after exposure to antigen, become functionally quiescent and which are capable of surviving for long periods in the absence of antigen. Human memory T cells are CD45 RA−.

As used herein, the term “effector T cell” includes T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation while Th2 cells provide help to B cells and are critical in the allergic response (Mosmann and Coffman, 1989, Annu. Rev. Immunol. 7, 145-173; Paul and Seder, 1994, Cell 76, 241-251; Arthur and Mason, 1986, J. Exp. Med. 163, 774-786; Paliard et al., 1988, J. Immunol. 141, 849-855; Finkelman et al., 1988, J. Immunol. 141, 2335-2341). As used herein, the term “T helper type 1 response” (Th1 response) refers to a response that is characterized by the production of one or more cytokines selected from IFN-γ, IL-2, TNF, and lymphotoxin (LT) and other cytokines produced preferentially or exclusively by Th1 cells rather than by Th2 cells.

As used herein, the term “regulatory T cell” includes T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at lower levels than in Th1 or Th2 cells, e.g., an order of magnitude less than in Th1 or Th2 cells. Regulatory T cells can be found in the CD4+ CD25+ population of cells (see, e.g. Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody plus anti-CD28 antibody).

As used herein, the term “dendritic cell” refers to a type of antigen-presenting cells which are particularly active in stimulating T cells. Dendritic cells can be obtained by culturing bone-marrow cells in the presence of GM-CSF and selecting those cells that express MHC class II molecules and CD11c. Dendritic cells can also express CD11b⁺, DEC-205⁺, CD8-alpha⁺.

As used herein, the term “immune response” includes T cell mediated and/or B cell mediated immune. Exemplary immune responses include T cell responses, e.g., cytokine production, and cellular cytotoxicity. In addition, the term immune response includes antibody production (humoral responses) and activation of cells of the innate immune system, e.g., cytokine responsive cells such as macrophages.

As used herein, the term “T helper type 1 response” refers to a response that is characterized by the production of one or more cytokines selected from IFN-γ, IL-2, TNF, and lymphtoxin (LT) and other cytokines produced preferentially or exclusively by Th1 cells rather than by Th2 cells.

As used herein, a “T helper type 2 response” (Th2 response) refers to a response by CD4⁺ T cells that is characterized by the production of one or more cytokines selected from IL-4, IL-5, IL-6 and IL-10, and that is associated with efficient B cell “help” provided by the Th2 cells (e.g., enhanced IgG1 and/or IgE production). As used herein, the term “a cytokine that regulates development of a Th2 response” is intended to include cytokines that have an effect on the initiation and/or progression of a Th2 response, in particular, cytokines that promote the development of a Th2 response, e.g., IL-4, IL-5 and IL-10.

Various aspects of the invention are described in further detail in the following subsections:

I. Isolated Nucleic Acid Molecules

One aspect of the invention pertains to isolated nucleic acid molecules that encode SLIM polypeptides or biologically active portions thereof, as well as nucleic acid fragments sufficient for use as hybridization probes to identify SLIM-encoding nucleic acid molecules (e.g., SLIM mRNA) and fragments for use as PCR primers for the amplification or mutation of SLIM nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid (i.e., free of sequences located at the 5′ and 3′ ends of the nucleic acid) in the genomic DNA of the organism from which the nucleic acid is derived. For example, in various embodiments, the isolated SLIM nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, or a portion thereof, can be isolated using standard molecular biology techniques and the sequence information provided herein. Using all or a portion of the nucleic acid sequence of SEQ ID NO:1 as a hybridization probe, SLIM nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1.

A nucleic acid molecule of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SLIM nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:1. In another embodiment, the nucleic acid molecule consists of the nucleotide sequence set forth as SEQ ID NO:1. In another embodiment, the nucleic acid molecule comprises the coding sequence of the nucleic acid molecule set forth in SEQ ID NO:1, or a complement thereof.

In still another embodiment, an isolated nucleic acid molecule of the invention comprises a nucleic acid molecule which is a complement of the nucleotide sequence shown in SEQ ID NO:1, or a portion of thereof. In one embodiment, the complement of the nucleotide sequence shown in SEQ ID NO:1, or a portion of thereof is an RNA molecule. A nucleic acid molecule which is complementary to the nucleotide sequence shown in SEQ ID NO:1 is one which is sufficiently complementary to the nucleotide sequence shown in SEQ ID NO:1 such that it can hybridize to the nucleotide sequence shown in SEQ ID NO:1, thereby forming a stable duplex, e.g., a double-stranded duplex.

In still another preferred embodiment, an isolated nucleic acid molecule of the present invention comprises a nucleotide sequence which is at least about 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to the nucleotide sequence shown in SEQ ID NO:1 (e.g., to the entire length of the nucleotide sequence), or a portion thereof, e.g., a portion encoding a LIM and/or PDZ domain.

Moreover, the nucleic acid molecules used in the methods of the invention can comprise only a portion of the nucleic acid sequence of SEQ ID NO:1, for example, a fragment which can be used as a probe or primer or a fragment encoding a portion of a SLIM polypeptide, e.g., a biologically active portion of a SLIM polypeptide. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 of an anti-sense sequence of SEQ ID NO:1 or of a SLIM family member. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to the complement of a nucleic acid molecule of SEQ ID NO:1 or a portion thereof encoding, for example a LIM and/or PDZ domain.

In one embodiment, a nucleic acid molecule of the present invention comprises a nucleotide sequence which is at least (or no greater than) 50, 100, 200, 250, 300, 350, 400, 450, 500, 550, 600, 650, 700, 750, 800, 850, 900, 950, 1,000, 1,500, 2,000, 2,500 or more nucleotides in length and hybridizes under stringent hybridization conditions to a complement of a nucleic acid molecule of SEQ ID NO:1.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70*C (or hybridization in 4×SSC plus 50% formamide at about 42-50*C) followed by one or more washes in 1×SSC, at about 65-70*C. A preferred, non-limiting example of highly stringent hybridization conditions includes hybridization in 1×SSC, at about 65-70*C (or hybridization in 1×SSC plus 50% formamide at about 42-50*C) followed by one or more washes in 0.3×SSC, at about 65-70*C. A preferred, non-limiting example of reduced stringency hybridization conditions includes hybridization in 4×SSC, at about 50-60*C (or alternatively hybridization in 6×SSC plus 50% formamide at about 40-45*C) followed by one or more washes in 2×SSC, at about 50-60*C. Ranges intermediate to the above-recited values, e.g., at 65-70*C or at 42-50*C are also intended to be encompassed by the present invention. SSPE (1×SSPE is 0.15M NaCl, 10 mM NaH2PO4, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log10[Na+])+0.41(% G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH2PO4, 7% SDS at about 65° C., followed by one or more washes at 0.02M NaH2PO4, 1% SDS at 65° C., see e.g., Church and Gilbert (1984) Proc. Natl. Acad. Sci. USA 81:1991-1995, (or alternatively 0.2×SSC, 1% SDS).

Moreover, the nucleic acid molecule of the invention can comprise only a portion of the nucleic acid sequence of SEQ ID NO:1, for example, a fragment which can be used as a probe or primer or a fragment encoding a portion of a SLIM polypeptide, e.g., a biologically active portion of a SLIM polypeptide. The nucleotide sequence determined from the cloning of the SLIM gene allows for the generation of probes and primers designed for use in identifying and/or cloning other SLIM family members, as well as SLIM homologues from other species. The probe/primer typically comprises substantially purified oligonucleotide. The probe/primer (e.g., oligonucleotide) typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, or 100 or more consecutive nucleotides of a sense sequence of SEQ ID NO:1, of an anti-sense sequence of SEQ ID NO:1, or of a naturally occurring allelic variant or mutant of SEQ ID NO:1.

Exemplary probes or primers are at least 12, 15, 20, 25, 30, 35, 40, 45, 50, 55, 60, 65, 70, 75 or more nucleotides in length and/or comprise consecutive nucleotides of an isolated nucleic acid molecule described herein. Probes based on the SLIM nucleotide sequences can be used to detect (e.g., specifically detect) transcripts or genomic sequences encoding the same or homologous polypeptides. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of a SLIM sequence, e.g., a domain, region, e.g., LIM and/or PDZ domain, site or other sequence described herein. The primers should be at least 5, 10, 20, 30, 40, 50, 60, 70, 80, 90, 100 or more nucleotides in length. Such probes can be used as a part of a diagnostic test kit for identifying cells or tissue which misexpress a SLIM polypeptide, such as by measuring a level of a SLIM-encoding nucleic acid in a sample of cells from a subject e.g., detecting SLIM mRNA levels or determining whether a genomic SLIM gene has been mutated or deleted.

A nucleic acid fragment encoding a “biologically active portion of a SLIM polypeptide” can be prepared by isolating a portion of the nucleotide sequence of SEQ ID NO:1, which encodes a polypeptide having a SLIM biological activity (the biological activities of the SLIM polypeptides are described herein), expressing the encoded portion of the SLIM polypeptide (e.g., by recombinant expression in vitro) and assessing the activity of the encoded portion of the SLIM polypeptide. In an exemplary embodiment, the nucleic acid molecule is at least 50, 100, 200, 250, 300, 350, 400, 450, 500, 550, 600, 650, 700, 750, 800, 850, 900, 950, 1,000, 1,500, 2,000, 2,500, or more nucleotides in length and encodes a polypeptide having a SLIM activity (as described herein). In one embodiment, the invention pertains to a polypeptide comprising at least one of a LIM and/or a PDZ domain. In one embodiment, such a polypeptide comprises a LIM and/or a PDZ consensus sequence. In another embodiment, such a polypeptide comprises a specific LIM and/or PDZ sequence set forth herein.

The invention further encompasses nucleic acid molecules that differ from the nucleotide sequence shown in SEQ ID NO:1. Such differences can be due to due to degeneracy of the genetic code, thus resulting in a nucleic acid which encodes the same SLIM polypeptides as those encoded by the nucleotide sequence shown in SEQ ID NO:1. In another embodiment, an isolated nucleic acid molecule of the invention has a nucleotide sequence encoding a polypeptide having an amino acid sequence which differs by at least 1, but no greater than 5, 10, 20, 50 or 100 amino acid residues from the amino acid sequence shown in SEQ ID NO:2. In yet another embodiment, the nucleic acid molecule encodes the amino acid sequence of rhesus monkey SLIM. If an alignment is needed for this comparison, the sequences should be aligned for maximum homology. In one embodiment, the polypeptide comprises a LIM and/or PDZ domain.

Nucleic acid variants can be naturally occurring, such as allelic variants (same locus), homologues (different locus), and orthologues (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Allelic variants result, for example, from DNA sequence polymorphisms within a population (e.g., the human population) that lead to changes in the amino acid sequences of the SLIM polypeptides. Such genetic polymorphism in the SLIM genes may exist among individuals within a population due to natural allelic variation. As used herein, the terms “gene” and “recombinant gene” refer to nucleic acid molecules which include an open reading frame encoding a SLIM polypeptide, preferably a mammalian SLIM polypeptide, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:2, wherein the nucleic acid molecule hybridizes to a complement of a nucleic acid molecule comprising SEQ ID NO:1, for example, under stringent hybridization conditions.

Allelic variants of SLIM include both functional and non-functional SLIM polypeptides. Functional allelic variants are naturally occurring amino acid sequence variants of the SLIM polypeptide that have a SLIM activity, e.g., maintain the ability to bind a SLIM binding partner and/or modulate STAT ubiquitination, modulate STAT phosphorylation, modulate IFN-γ production, modulate STAT signaling, modulate Th1 cell differentiation, modulate protein folding, protein transport, and/or protein secretion, and modulate protein degradation. Functional allelic variants will typically contain only conservative substitution of one or more amino acids of SEQ ID NO:2, or substitution, deletion or insertion of non-critical residues in non-critical regions of the polypeptide.

Non-functional allelic variants are naturally occurring amino acid sequence variants of the SLIM polypeptide that do not have a SLIM activity, e.g., they do not have the ability to bind a SLIM binding partner and/modulate STAT ubiquitination, modulate STAT phosphorylation, modulate IFN-γ production, modulate STAT signaling, modulate Th1 cell differentiation, modulate protein folding, protein transport, and/or protein secretion, and modulate protein degradation. Non-functional allelic variants will typically contain a non-conservative substitution, a deletion, or insertion or premature truncation of the amino acid sequence of SEQ ID NO:2, or a substitution, insertion or deletion in critical residues or critical regions.

Nucleic acid molecules encoding other SLIM family members and, thus, which have a nucleotide sequence which differs from the SLIM sequence of SEQ ID NO:1 are intended to be within the scope of the invention. For example, another SLIM cDNA can be identified based on the nucleotide sequence of murine SLIM. Moreover, nucleic acid molecules encoding SLIM polypeptides from different species, and which, thus, have a nucleotide sequence which differs from the SLIM sequences of SEQ ID NO:1 are intended to be within the scope of the invention. For example, a human SLIM cDNA can be identified based on the nucleotide sequence of a murine SLIM. As described above, human, rat and murine SLIM family members are known.

Nucleic acid molecules corresponding to natural allelic variants and homologues of the SLIM cDNAs of the invention can be isolated based on their homology to the SLIM nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the SLIM cDNAs of the invention can further be isolated by mapping to the same chromosome or locus as the SLIM gene.

Orthologues, homologues and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1. In other embodiment, the nucleic acid is at least 50, 100, 200, 250, 300, 350, 400, 450, 500 or more nucleotides in length.

Preferably, an isolated nucleic acid molecule of the invention that hybridizes under stringent conditions to the sequence of SEQ ID NO:1 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural polypeptide).

In addition to naturally-occurring allelic variants of the SLIM sequences that may exist in the population, the skilled artisan will further appreciate that changes can be introduced by mutation into the nucleotide sequences of SEQ ID NO:1, thereby leading to changes in the amino acid sequence of the encoded SLIM polypeptides, without altering the functional ability of the SLIM polypeptides. For example, nucleotide substitutions leading to amino acid substitutions at “non-essential” amino acid residues can be made in the sequence of SEQ ID NO:1. A “non-essential” amino acid residue is a residue that can be altered from the wild-type sequence of SLIM (e.g., the sequence of SEQ ID NO:1) without altering the biological activity, whereas an “essential” amino acid residue is required for biological activity. For example, amino acid residues that are conserved among the SLIM polypeptides of the present invention, e.g., those present in a LIM and/or PDZ domain, are predicted to be particularly unamenable to alteration. Furthermore, additional amino acid residues that are conserved between the SLIM polypeptides of the present invention and other members of the SLIM family are not likely to be amenable to alteration.

Accordingly, another aspect of the invention pertains to nucleic acid molecules encoding SLIM polypeptides that contain changes in amino acid residues that are not essential for activity. Such SLIM polypeptides differ in amino acid sequence from SEQ ID NO:2, yet retain biological activity. In one embodiment, the isolated nucleic acid molecule comprises a nucleotide sequence encoding a polypeptide, wherein the polypeptide comprises an amino acid sequence at least about 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO:2 (e.g., to the entire length of SEQ ID NO:2).

An isolated nucleic acid molecule encoding a SLIM polypeptide identical to or comprising one or more mutation in a non-essential amino acid of SEQ ID NO:2, can be created by introducing one or more nucleotide substitutions, additions or deletions into the nucleotide sequence of SEQ ID NO:1, such that one or more amino acid substitutions, additions or deletions are introduced into the encoded polypeptide. Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a SLIM polypeptide is preferably replaced with another amino acid residue from the same side chain family. Alternatively, in another embodiment, mutations can be introduced randomly along all or part of a SLIM coding sequence, such as by saturation mutagenesis, and the resultant mutants can be screened for SLIM biological activity to identify mutants that retain activity. Following mutagenesis of SEQ ID NO:1, the encoded polypeptide can be expressed recombinantly and the activity of the polypeptide can be determined.

In a preferred embodiment, a mutant SLIM polypeptide can be assayed for the ability to 1) bind to one or more STAT molecules, 2) modulate STAT ubiquitination, 3) modulate STAT phosphorylation, 4) modulate IFN-γ production, 5) modulate STAT signaling, 6) modulate Th1 cell differentiation, 7) modulate protein folding, protein transport, and/or protein secretion, and 8) modulate protein degradation, using techniques known in the art and as described in more detail herein.

In addition to the nucleic acid molecules encoding SLIM polypeptides described above, another aspect of the invention pertains to isolated nucleic acid molecules which are antisense thereto. In an exemplary embodiment, the invention provides an isolated nucleic acid molecule which is antisense to a SLIM nucleic acid molecule (e.g., is antisense to the coding strand of a SLIM nucleic acid molecule). An “antisense” nucleic acid comprises a nucleotide sequence which is complementary to a “sense” nucleic acid encoding a polypeptide, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire SLIM coding strand, or to only a portion thereof. In one embodiment, an antisense nucleic acid molecule is antisense to a “coding region” of the coding strand of a nucleotide sequence encoding SLIM. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding SLIM. The term “noncoding region” refers to 5′ and 3′ sequences which flank the coding region that are not translated into amino acids (i.e., also referred to as 5′ and 3′ untranslated regions).

Given the coding strand sequences encoding SLIM disclosed herein (e.g., nucleic acids 68-1047 of SEQ ID NO:1), antisense nucleic acids of the invention can be designed according to the rules of Watson and Crick base pairing. The antisense nucleic acid molecule can be complementary to the entire coding region of SLIM mRNA, but more preferably is an oligonucleotide which is antisense to only a portion of the coding or noncoding region of SLIM mRNA. For example, the antisense oligonucleotide can be complementary to the region surrounding the translation start site of SLIM mRNA (e.g., between the 10 and −10 regions of the start site of a gene nucleotide sequence). An antisense oligonucleotide can be, for example, about 5, 10, 15, 20, 25, 30, 35, 40, 45 or 50 nucleotides in length. Preferably, an antisense nucleic acid is designed so as to be complementary to a region preceding or spanning the initiation codon on the coding strand or in the 3′ untranslated region of an mRNA. An antisense nucleic acid for inhibiting the expression of a SLIM polypeptide in a cell can be designed based upon the nucleotide sequence encoding the a SLIM polypeptide, constructed according to the rules of Watson and Crick base pairing.

An antisense nucleic acid can exist in a variety of different forms. For example, the antisense nucleic acid can be an oligonucleotide that is complementary to only a portion of a SLIM gene. To inhibit the expression of a SLIM in cells in culture, one or more antisense oligonucleotides can be added to cells in culture media, typically at about 200 μg oligonucleotide/ml.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, I-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Alternatively, an antisense nucleic acid can be produced biologically using an expression vector into which a nucleic acid has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. For example, for inducible expression of antisense RNA, an inducible eukaryotic regulatory system, such as the Tet system (e.g., as described in Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313) can be used. The antisense expression vector is prepared as described above for recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique, as described above for recombinant expression vectors.

Given the coding strand sequences encoding SLIM family members disclosed herein, antisense nucleic acids of the invention can be designed according to the rules of Watson and Crick base pairing. The antisense nucleic acid molecule can be complementary to the entire coding region of a SLIM mRNA, but more preferably is an oligonucleotide which is antisense to only a portion of the coding or noncoding region of a SLIM mRNA. For example, the antisense oligonucleotide can be complementary to the region surrounding the translation start site of a SLIM mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides may be used. Examples of modified nucleotides which may be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a SLIM protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier, et al. (1987) Nucleic Acids. Res. 15:6625-6641). The antisense nucleic acid molecule can also comprise a 2′-o-methylribonucleotide (Inoue, et al. (11987) Nucleic Acids Res. 15:6131-6148) or a chimeric RNA-DNA analogue (Inoue, et al. (11987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave SLIM mRNA transcripts to thereby inhibit translation of SLIM mRNA. A ribozyme having specificity for a SLIM-encoding nucleic acid can be designed based upon the nucleotide sequence of a SLIM cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a SLIM-encoding mRNA. See, e.g., Cech, et al. U.S. Pat. No. 4,987,071; and Cech, et al. U.S. Pat. No. 5,116,742. Alternatively, SLIM mRNA can be used to select a catalytic RNA having, a specific ribonuclease activity from a pool of RNA molecules. See, e.g. Bartel, D. and Szostak, J. W. (11993) Science 261:1411-1418.

Alternatively, SLIM genie expression can be inhibited by targeting, nucleotide sequences complementary to the regulatory region of the SLIM (e.g., the SLIM promoter and/or enhancers) to form triple helical structures that prevent transcription of the SLIM genie in target cells. See generally, Helene. C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the SLIM nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, B. et al. (1996) supra; Perry-O'Keefe, et al. Proc. Natl. Acad. Sci., USA 93: 14670-675.

PNAs of SLIM nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of SLIM nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup. B. (1996) supra)); or as probes or primers for DNA sequencing or hybridization (Hyrup, B. et al. (1996) supra; Perry-O'Keefe supra).

In another embodiment, PNAs of SLIM can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of SLIM nucleic acid molecules can be generated which may combine the advantageous properties of PNA and DNA. Such chimeras allow DNA recognition enzymes, (e.g., RNase H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup, B. (1996) supra and Finn P. J., et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M., et al. (1989) Nucleic Acids Res. 17: 5973-88). PNA monomers are then coupled in a stepwise manner to produce a chimeric molecule with a 5′ PNA segment and a 3′ DNA segment (Finn, P. J., et al. (1996) supra). Alternatively, chimeric molecules can be synthesized with a 5′ DNA segment and a 3′ PNA segment (Peterser, K. H., et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger, et al. (1989) Proc. Natl. Acad. Sci., USA 86:6553-6556; Lemaitre, et al. (1987) Proc. Natl. Acad. Sci., USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol, et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous SLIM gene within a cell line or microorganism may be modified by inserting a heterologous DNA regulatory element into the genome of a stable cell line or cloned microorganism such that the inserted regulatory element is operatively linked with the endogenous SLIM gene. For example, an endogenous SLIM gene which is normally “transcriptionally silent”, i.e., a SLIM gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous SLIM gene may be activated by insertion of a promiscuous regulatory element that works across cell types.

A heterologous regulatory element may be inserted into a stable cell line or cloned microorganism, such that it is operatively linked with an endogenous SLIM gene, using techniques, such as targeted homologous recombination, which are well known to those of skill in the art, and described, e.g., in Chappel, U.S. Pat. No. 5,272,071; PCT Publication No. WO 91/06667, published May 16, 1991.

II. Isolated SLIM Polypeptides and Anti-SLIM Antibodies

One aspect of the invention pertains to isolated SLIM or recombinant polypeptides and polypeptides, and biologically active portions thereof, as well as polypeptide fragments suitable for use as immunogens to raise anti-SLIM antibodies. In one embodiment, native SLIM polypeptides can be isolated from cells or tissue sources by an appropriate purification scheme using standard protein purification techniques. In another embodiment, SLIM polypeptides are produced by recombinant DNA techniques. Alternative to recombinant expression, a SLIM polypeptide or polypeptide can be synthesized chemically using standard peptide synthesis techniques.

An “isolated” or “purified” polypeptide or biologically active portion thereof is substantially free of cellular material or other contaminating proteins from the cell or tissue source from which the SLIM polypeptide is derived, or substantially free from chemical precursors or other chemicals when chemically synthesized. The language “substantially free of cellular material” includes preparations of SLIM polypeptide in which the polypeptide is separated from cellular components of the cells from which it is isolated or recombinantly produced. In one embodiment, the language “substantially free of cellular material” includes preparations of SLIM polypeptide having less than about 30% (by dry weight) of non-SLIM polypeptide (also referred to herein as a “contaminating protein”), more preferably less than about 20% of non-SLIM polypeptide, still more preferably less than about 10% of non-SLIM polypeptide, and most preferably less than about 5% non-SLIM polypeptide. When the SLIM polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of SLIM polypeptide in which the polypeptide is separated from chemical precursors or other chemicals which are involved in the synthesis of the polypeptide. In one embodiment, the language “substantially free of chemical precursors or other chemicals” includes preparations of SLIM polypeptide having less than about 30% (by dry weight) of chemical precursors or non-SLIM chemicals, more preferably less than about 20% chemical precursors or non-SLIM chemicals, still more preferably less than about 10% chemical precursors or non-SLIM chemicals, and most preferably less than about 5% chemical precursors or non-SLIM chemicals.

As used herein, a “biologically active portion” of a SLIM polypeptide includes a fragment of a SLIM polypeptide which participates in an interaction between a SLIM molecule and a non-SLIM molecule, and/or which mediates a SLIM downstream cellular signaling event. Biologically active portions of a SLIM polypeptide include amino acid sequences sufficiently identical to or derived from the amino acid sequence of the SLIM polypeptide, e.g., the amino acid sequence shown in SEQ ID NO:2, which include less amino acids than the full length SLIM polypeptides, and exhibit at least one activity of a SLIM polypeptide. Typically, biologically active portions comprise a domain or motif with at least one activity of the SLIM polypeptide, e.g., modulation of STAT ubiquitination, modulation of STAT phosphorylation, modulation of IFN-γ production, modulation of STAT signaling, modulation of Th1 cell differentiation, modulation of protein folding, protein transport, and/or protein secretion, and modulation of protein degradation. A biologically active portion of a SLIM polypeptide can be a polypeptide which is, for example, 25, 30, 35, 40, 45, 50, 55, 60, 65, 70, 75, 80, 100, or more amino acids in length. Biologically active portions of a SLIM polypeptide can be used as targets for developing agents which modulate a SLIM mediated activity.

In one embodiment, a biologically active portion of a SLIM polypeptide comprises at least one PDZ domain. In another embodiment, a biologically active portion of a SLIM polypeptide comprises at least one LIM domain. It is to be understood that a preferred biologically active portion of a SLIM polypeptide of the present invention comprises at least one or more of the following domains: a LIM domain, and/or a PDZ domain. Moreover, other biologically active portions, in which other regions of the polypeptide are deleted, can be prepared by recombinant techniques and evaluated for one or more of the functional activities of a native SLIM polypeptide.

Another aspect of the invention features fragments of a SLIM polypeptide, for example, for use as immunogens. In one embodiment, a fragment comprises at least 8 amino acids (e.g., contiguous or consecutive amino acids) of the amino acid sequence of SEQ ID NO:2. In another embodiment, a fragment comprises at least 10, 15, 20, 25, 30, 35, 40, 45, 50 or more amino acids (e.g., contiguous or consecutive amino acids) of the amino acid sequence of SEQ ID NO:2.

In a preferred embodiment, a SLIM polypeptide has an amino acid sequence comprising SEQ ID NO:2. In other embodiments, the SLIM polypeptide consists of the amino acid sequence of SEQ ID NO:2. In other embodiments, the SLIM polypeptide comprising an amino acid sequence structurally related to SEQ ID NO:2, which retains a functional activity of the polypeptide of SEQ ID NO:2, yet differs in amino acid sequence, e.g., due to natural allelic variation or mutagenesis, as described in detail in subsection I above. In another embodiment, the SLIM polypeptide is a polypeptide which comprises an amino acid sequence at least about 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO:2. In a preferred embodiment, the SLIM polypeptide is a polypeptide which comprises an amino acid sequence that has at least 95% identity to SEQ ID NO:2 and binds to a STAT molecule, e.g., STAT4 and/or STAT1. In another preferred embodiment, the SLIM polypeptide is a polypeptide which comprises an amino acid sequence that has at least 95% identity across the full-length of the polypeptide set forth in SEQ ID NO:2

In another embodiment, the invention features a SLIM polypeptide which is encoded by a nucleic acid molecule consisting of a nucleotide sequence at least about 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to a nucleotide sequence of SEQ ID NO:1, or a complement thereof. This invention further features a SLIM polypeptide which is encoded by a nucleic acid molecule consisting of a nucleotide sequence which hybridizes under stringent hybridization conditions to a complement of a nucleic acid molecule comprising the nucleotide sequence of SEQ ID NO:1, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence (e.g., when aligning a second sequence to the SLIM amino acid sequence of SEQ ID NO:2). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0 or version 2.0U), using a PAM120 weight residue table, a gap length penalty of 12 and a gap penalty of 4.

The nucleic acid and polypeptide sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to SLIM nucleic acid molecules of the invention. BLAST protein searches can be performed with the XBLAST program, score=100, wordlength=3, and a Blosum62 matrix to obtain amino acid sequences homologous to SLIM polypeptide molecules of the invention. To obtain gapped alignments for comparison purposes, Gapped BLAST can be utilized as described in Altschul, et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides SLIM chimeric or fusion proteins. In one embodiment, a SLIM “chimeric protein” or “fusion protein” comprises a SLIM polypeptide operatively linked to a non-SLIM polypeptide, e.g., a heterologous polypeptide. A “SLIM polypeptide” refers to a polypeptide having an amino acid sequence derived from to a SLIM protein, whereas a “non-SLIM polypeptide” refers to a polypeptide having an SLIM acid sequence corresponding to a polypeptide which is not substantially homologous to the SLIM polypeptide, e.g., a polypeptide which is different from the SLIM polypeptide, and which is derived from the same or a different organism. Within a SLIM fusion protein the SLIM polypeptide can correspond to all or a portion of a SLIM polypeptide. In one embodiment, a SLIM fusion protein comprises one or more of a LIM and/or PDZ domain, e.g., a portion of a SLIM which has a SLIM biological activity, and a non-SLIM polypeptide. In a preferred embodiment, a SLIM fusion protein comprises at least one biologically active portion of a SLIM polypeptide. In another preferred embodiment, a SLIM fusion protein comprises at least two biologically active portions of a SLIM polypeptide. Within the fusion protein, the term “operatively linked” is intended to indicate that the SLIM polypeptide and the non-SLIM polypeptide are fused in-frame to each other. The non-SLIM polypeptide can be fused to the N-terminus or C-terminus of the SLIM polypeptide.

For example, in one embodiment, the fusion protein is a GST-SLIM fusion protein in which the SLIM sequences are fused to the C-terminus of the GST sequences. Such fusion proteins can facilitate the purification of recombinant SLIM.

In another embodiment, the fusion protein is a SLIM polypeptide containing a heterologous signal sequence at its N-terminus. In certain host cells (e.g., mammalian host cells), expression and/or secretion of SLIM can be increased through the use of a heterologous signal sequence.

The SLIM fusion proteins of the invention can be incorporated into pharmaceutical compositions and administered to a subject in vivo. The SLIM fusion proteins can be used to affect the bioavailability of a SLIM substrate. Use of SLIM fusion proteins may be useful therapeutically for the treatment of disorders caused by, for example, (i) aberrant modification or mutation of a gene encoding a SLIM polypeptide; (ii) mis-regulation of the SLIM gene; and (iii) aberrant post-translational modification of a SLIM polypeptide.

Moreover, the SLIM-fusion proteins of the invention can be used as immunogens to produce anti-SLIM antibodies in a subject, to purify SLIM ligands and in screening assays to identify molecules which inhibit the interaction of SLIM with a SLIM substrate.

Preferably, a SLIM chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel, et al. John Wiley & Sons: 1992). Moreover, many expression vectors are commercially available that already encode a fusion moiety (e.g., a GST polypeptide). A SLIM-encoding nucleic acid can be cloned into such an expression vector such that the fusion moiety is linked in-frame to the SLIM polypeptide.

The present invention also pertains to variants of the SLIM polypeptides which function as either SLIM agonists (mimetics) or as SLIM antagonists. Variants of the SLIM polypeptides can be generated by mutagenesis, e.g., discrete point mutation or truncation of a SLIM polypeptide. An agonist of the SLIM polypeptides can retain substantially the same, or a subset, of the biological activities of the naturally occurring form of a SLIM polypeptide. An antagonist of a SLIM polypeptide can inhibit one or more of the activities of the naturally occurring form of the SLIM polypeptide by, for example, competitively modulating a SLIM-mediated activity of a SLIM polypeptide. For example, a SLIM antagonist can compete with SLIM for binding to STAT, e.g., STAT4 and/or STAT1. Thus, specific biological effects can be elicited by treatment with a variant of limited function.

In one embodiment, variants of a SLIM polypeptide which function as either SLIM agonists (mimetics) or as SLIM antagonists can be identified by screening combinatorial libraries of mutants, e.g., truncation mutants or mutants containing point mutations, of a SLIM polypeptide for SLIM polypeptide agonist or antagonist activity. In one embodiment, a variegated library of SLIM variants is generated by combinatorial mutagenesis at the nucleic acid level and is encoded by a variegated gene library. A variegated library of SLIM variants can be produced by, for example, enzymatically ligating a mixture of synthetic oligonucleotides into gene sequences such that a degenerate set of potential SLIM sequences is expressible as individual polypeptides, or alternatively, as a set of larger fusion proteins (e.g., for phage display) containing the set of SLIM sequences therein. There are a variety of methods which can be used to produce libraries of potential SLIM variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SLIM sequences. Methods for synthesizing degenerate oligonucleotides are known in the art (see, e.g., Narang, S. A. (1983) Tetrahedron 39:3; Itakura, et al. (1984) Annu Rev. Biochem. 53:323; Itakura, et al. (1984) Science 198:1056; Ike, et al. (1983) Nucleic Acids Res. 11:477.

In addition, libraries of fragments of a SLIM polypeptide coding sequence can be used to generate a variegated population of SLIM fragments for screening and subsequent selection of variants of a SLIM polypeptide. In one embodiment, a library of coding sequence fragments can be generated by treating a double stranded PCR fragment of a SLIM coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the SLIM polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SLIM polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify SLIM variants (Arkin and Yourvan (1992) Proc. Natl. Acad. Sci., USA 89:7811-7815; Delgrave, et al. (1993) Protein Engineering 6(3):327-331).

In one embodiment, cell based assays can be exploited to analyze a variegated SLIM library. For example, a library of expression vectors can be transfected into a cell line, e.g., a T cell line (such as a Th1 cell line), which ordinarily responds to SLIM in a particular SLIM substrate-dependent manner. The transfected cells are then contacted with SLIM and the effect of expression of the mutant on signaling by the SLIM substrate can be detected, e.g., by monitoring ubiquitination. Plasmid DNA can then be recovered from the cells which score for inhibition, or alternatively, potentiation of signaling by the SLIM substrate, and the individual clones further characterized.

An isolated SLIM polypeptide, or a portion or fragment thereof, can be used as an immunogen to generate antibodies that bind SLIM using standard techniques for polyclonal and monoclonal antibody preparation. A full-length SLIM polypeptide can be used or, alternatively, the invention provides antigenic peptide fragments of SLIM for use as immunogens. The antigenic peptide of SLIM comprises at least 8 amino acid residues of the amino acid sequence shown in SEQ ID NO:2 and encompasses an epitope of SLIM such that an antibody raised against the peptide forms a specific immune complex with SLIM. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of SLIM that are located on the surface of the polypeptide, e.g., hydrophilic regions, as well as regions with high antigenicity.

A SLIM immunogen typically is used to prepare antibodies by immunizing a suitable subject, (e.g., rabbit, goat, mouse or other mammal) with the immunogen. An appropriate immunogenic preparation can contain, for example, recombinantly expressed SLIM polypeptide or a chemically synthesized SLIM polypeptide. The preparation can further include an adjuvant, such as Freund's complete or incomplete adjuvant, or similar immunostimulatory agent. Immunization of a suitable subject with an immunogenic SLIM preparation induces a polyclonal anti-SLIM antibody response.

Accordingly, another aspect of the invention pertains to anti-SLIM antibodies. The term “antibody” as used herein refers to immunoglobulin molecules and immunologically active portions of immunoglobulin molecules, i.e., molecules that contain an antigen binding site which specifically binds (immunoreacts with) an antigen, such as SLIM. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind SLIM. The term “monoclonal antibody” or “monoclonal antibody composition”, as used herein, refers to a population of antibody molecules that contain only one species of an antigen binding site capable of immunoreacting with a particular epitope of SLIM. A monoclonal antibody composition thus typically displays a single binding affinity for a particular SLIM polypeptide with which it immunoreacts.

Polyclonal anti-SLIM antibodies can be prepared as described above by immunizing a suitable subject with a SLIM immunogen or a nucleic acid molecule encoding the same. The anti-SLIM antibody titer in the immunized subject can be monitored over time by standard techniques, such as with an enzyme linked immunosorbent assay (ELISA) using immobilized SLIM. If desired, the antibody molecules directed against SLIM can be isolated from the mammal (e.g., from the blood) and further purified by well known techniques, such as protein A chromatography to obtain the IgG fraction. At an appropriate time after immunization, e.g., when the anti-SLIM antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown, et al. (1981) J. Immunol. 127:539-46; Brown, et al. (1980) J. Biol. Chem 255:4980-83; Yeh, et al. (1976) Proc. Natl. Acad. Sci., USA 76:2927-31; and Yeh, et al. (1982) Int. J. Cancer 29:269-75), the more recent human B cell hybridoma technique (Kozbor, et al. (1983) Immunol Today 4:72), the EBV-hybridoma technique (Cole, et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter, et al. (1977) Somatic Cell Genet. 3:231-36). Briefly, an immortal cell line (typically a myeloma) is fused to lymphocytes (typically splenocytes) from a mammal immunized with a SLIM immunogen as described above, and the culture supernatants of the resulting hybridoma cells are screened to identify a hybridoma producing a monoclonal antibody that binds SLIM.

Any of the many well known protocols used for fusing lymphocytes and immortalized cell lines can be applied for the purpose of generating an anti-SLIM monoclonal antibody (see, e.g., G. Galfre, et al. (1977) Nature 266:55052; Gefter, et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind SLIM, e.g., using a standard ELISA assay.

Alternative to preparing monoclonal antibody-secreting hybridomas, a monoclonal anti-SLIM antibody can be identified and isolated by screening a recombinant combinatorial immunoglobulin library (e.g., an antibody phage display library) with SLIM to thereby isolate immunoglobulin library members that bind SLIM. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner, et al. U.S. Pat. No. 5,223,409; Kang, et al. PCT International Publication No. WO 92/18619; Dower, et al. PCT International Publication No. WO 91/17271; Winter, et al. PCT International Publication WO 92/20791; Markland, et al. PCT International Publication No. WO 92/15679; Breitling, et al. PCT International Publication WO 93/01288; McCafferty, et al. PCT International Publication No. WO 92/01047; Garrard, et al. PCT International Publication No. WO 92/09690; Ladner, et al. PCT International Publication No. WO 90/02809; Fuchs, et al. (1991) Bio/Technology; 9: 1370-1372; Hay, et al. (1992) Hum. Antibod. Hybridomas 3:81-85; Huse, et al. (1989) Science 246:1275-1281; Griffiths, et al. (1993) EMBO J. 12:725-734; Hawkins, et al. (1992) J. Mol. Biol. 226:889-896; Clarkson, et al. (1991) Nature 352:624-628; Gram, et al. (1992) Proc. Natl. Acad. Sci., USA 89:3576-3580; Garrad, et al. (1991) Bio/Technology 9:1373-1377; Hoogenboom, et al. (1991) Nuc. Acids Res. 19:4133-4137; Barbas, et al. (1991) Proc. Natl. Acad. Sci., USA 88:7978-7982; and McCafferty, et al. Nature (1990) 348:552-554.

Additionally, recombinant anti-SLIM antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson, et al. International Application No. PCT/US86/02269; Akira, et al. European Patent Application 184,187; Taniguchi, M., European Patent Application 171,496; Morrison et al. European Patent Application 173,494; Neuberger, et al. PCT International Publication No. WO 86/01533; Cabilly, et al. U.S. Pat. No. 4,816,567; Cabilly, et al. European Patent Application 125,023; Better, et al. (1988) Science 240:1041-1043; Liu, et al. (1987) Proc. Natl. Acad. Sci., USA 84:3439-3443; Liu, et al. (1987) J. Immunol. 139:3521-3526; Sun, et al. (1987) Proc. Natl. Acad. Sci., USA 84:214-218; Nishimura, et al. (1987) Canc. Res. 47:999-1005; Wood, et al. (1985) Nature 314:446-449; and Shaw, et al. (1988) J. Natl. Cancer Inst. 80:1553-1559); Morrison, S. L. (1985) Science 229:1202-1207; Oi, et al. (1986) BioTechniques 4:214; Winter, U.S. Pat. No. 5,225,539; Jones, et al. (1986) Nature 321:552-525; Verhoeyan, et al. (1988) Science 239:1534; and Beidler, et al. (1988) J. Immunol. 141:4053-4060. In one embodiment, an anti-SLIM antibody is a fully human antibody.

An anti-SLIM antibody (e.g., monoclonal antibody) can be used to isolate SLIM by standard techniques, such as affinity chromatography or immunoprecipitation. An anti-SLIM antibody can facilitate the purification of natural SLIM from cells and of recombinantly produced SLIM expressed in host cells. Moreover, an anti-SLIM antibody can be used to detect SLIM polypeptide (e.g., in a cellular lysate or cell supernatant) in order to evaluate the abundance and pattern of expression of the SLIM polypeptide. Anti-SLIM antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Expression Vectors and Host Cells

Another aspect of the invention pertains to vectors, for example expression vectors, containing a nucleic acid containing a SLIM nucleic acid molecule or vectors containing a nucleic acid molecule which encodes a SLIM polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology; 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, and the like. The expression vectors of the invention can be introduced into host cells to thereby produce proteins or peptides of the invention, including fusion proteins or peptides, encoded by nucleic acids as described herein (e.g., SLIM polypeptides, mutant forms of SLIM polypeptides, fusion proteins, and the like).

Accordingly, an exemplary embodiment provides a method for producing a polypeptide of the invention, by culturing in a suitable medium a host cell of the invention (e.g., a mammalian host cell such as a non-human mammalian cell) containing a recombinant expression vector, such that the polypeptide is produced. The recombinant expression vectors of the invention can be designed for expression of SLIM polypeptides in prokaryotic or eukaryotic cells. For example, SLIM polypeptides can be expressed in bacterial cells such as E. coli, insect cells (using baculovirus expression vectors) yeast cells or mammalian cells. Suitable host cells are discussed further in Goeddel, Gene Expression Technology. Methods in Enzymology; 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in SLIM activity assays, (e.g., direct assays or competitive assays described in detail below), or to generate antibodies specific for SLIM polypeptides, for example. In a preferred embodiment, a SLIM fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann, et al., (1988) Gene 69:301-315) and pET 11d (Studier, et al., Gene Expression Technology: Methods in Enzymology; 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada, et al., (1992) Nucleic Acids Res. 20:2111-2118). Such alteration of nucleic acid sequences of the invention can be carried out by standard DNA synthesis techniques.

In another embodiment, the SLIM expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz, et al., (1987) Gene 54:113-123), pYES2 (Invitrogen Corporation, San Diego, Calif.), and picZ (InVitrogen Corp, San Diego, Calif.).

Alternatively, SLIM polypeptides can be expressed in insect cells using baculovirus expression vectors. Baculovirus vectors available for expression of proteins in cultured insect cells (e.g., Sf 9 cells) include the pAc series (Smith, et al. (1983) Mol. Cell Biol. 3:2156-2165) and the pVL series (Lucklow and Summers (1989) Virology 170:31-39).

In yet another embodiment, a nucleic acid molecule of the invention is expressed in mammalian cells using a mammalian expression vector. Examples of mammalian expression vectors include pCDM8 (Seed, B. (1987) Nature 329:840) and pMT2PC (Kaufman, et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid molecule preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji, et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci., USA 86:5473-5477), pancreas-specific promoters (Edlund, et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to SLIM mRNA. Regulatory sequences operatively linked to a nucleic acid molecule cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a SLIM nucleic acid molecule of the invention is introduced, e.g., a SLIM nucleic acid molecule within a vector (e.g., a recombinant expression vector) or a SLIM nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a SLIM polypeptide can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning. A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a SLIM polypeptide or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a SLIM polypeptide using the host cells of the invention. In one embodiment, the method comprises culturing the host cell of the invention (into which a recombinant expression vector encoding, e.g., a SLIM polypeptide has been introduced) in a suitable medium such that the polypeptide is produced. The invention also provides methods for producing a polypeptide that binds STAT, e.g., STAT4 and/or STAT1, using the host cells of the invention. In another embodiment, the method further comprises isolating a polypeptide of the invention from the medium or the host cell.

The host cells of the invention can also be used to produce non-human transgenic animals. For example, in one embodiment, a host cell of the invention is a fertilized oocyte or an embryonic stem cell into which SLIM-coding sequences have been introduced. Such host cells can then be used to create non-human transgenic animals in which exogenous SLIM sequences have been introduced into their genome or homologous recombinant animals in which endogenous SLIM sequences have been altered. Such animals are useful for studying the function and/or activity of a SLIM and for identifying and/or evaluating modulators of SLIM activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SLIM gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a SLIM-encoding nucleic acid into the male pronuclei of a fertilized oocyte, e.g., by microinjection, retroviral infection, and allowing the oocyte to develop in a pseudopregnant female foster animal. The SLIM cDNA sequence of SEQ ID NO:1 can be introduced as a transgene into the genome of a non-human animal. Alternatively, a SLIM gene homologue, such as another SLIM family member, can be isolated based on hybridization to the SLIM cDNA sequences of SEQ ID NO:1 (described further in subsection I above) and used as a transgene. Intronic sequences and polyadenylation signals can also be included in the transgene to increase the efficiency of expression of the transgene. A tissue-specific regulatory sequence(s) can be operably linked to a SLIM transgene to direct expression of a SLIM polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder, et al., U.S. Pat. No. 4,873,191 by Wagner, et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a SLIM transgene in its genome and/or expression of SLIM mRNA in tissues or cells of the animals. A transgenic founder animal can then be used to breed additional animals carrying the transgene. Moreover, transgenic animals carrying a transgene encoding a SLIM polypeptide can further be bred to other transgenic animals carrying other transgenes.

To create a homologous recombinant animal, a vector is prepared which contains at least a portion of a SLIM gene into which a deletion, addition or substitution has been introduced to thereby alter, e.g., functionally disrupt, the SLIM gene. The SLIM gene can be a murine gene (e.g., SEQ ID NO:1), or a homologue thereof. In a preferred embodiment, the homologous recombination nucleic acid molecule is designed such that, upon homologous recombination, the endogenous SLIM gene is functionally disrupted (i.e., no longer encodes a functional protein; also referred to as a “knock-out” vector). Alternatively, the homologous recombination nucleic acid molecule can be designed such that, upon homologous recombination, the endogenous SLIM gene is mutated or otherwise altered but still encodes functional polypeptide (e.g., the upstream regulatory region can be altered to thereby alter the expression of the endogenous SLIM polypeptide). In the homologous recombination nucleic acid molecule, the altered portion of the SLIM gene is flanked at its 5′ and 3′ ends by additional nucleic acid sequence of the SLIM gene to allow for homologous recombination to occur between the exogenous SLIM gene carried by the homologous recombination nucleic acid molecule and an endogenous SLIM gene in a cell, e.g., an embryonic stem cell. The additional flanking SLIM nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced SLIM gene has homologously recombined with the endogenous SLIM gene are selected (see e.g., Li, E., et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their gem cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec, et al.; WO 91/01140 by Smithies, et al.; WO 92/0968 by Zijlstra, et al.; and WO 93/04169 by Berns, et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso, et al. (1992) Proc. Natl. Acad. Sci. USA 89:6232-6236. Another example of a recombinase system is the FLP recombinase system of Saccharomyces cerevisiae (O'Gorman, et al. (1991) Science 251:1351-11355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I., et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The nucleic acid molecules, polypeptides, antibodies, or portions thereof, or other modulating compounds of the invention can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a SLIM polypeptide of the invention has one or more of the following activities: 1) binding to STAT, 2) modulation of STAT ubiquitination, 3) modulation of STAT phosphorylation, 4) modulation of IFN-γ production, 5) modulation of STAT signaling, 6) modulation of Th1 cell differentiation, 7) modulation of protein folding, protein transport, and/or protein secretion, and 8) modulation of protein degradation.

The nucleic acid molecules, polypeptides, antibodies, or portions thereof, or other modulating compounds of the invention (also referred to herein as “active compounds”) of the invention can be incorporated into pharmaceutical compositions suitable for administration. Such compositions typically comprise the nucleic acid molecule, or polypeptide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, vaginal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., SLIM nucleic acid molecules, a fragment of a SLIM polypeptide or an anti-SLIM antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid. Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery. Vaginal suppositories or foams for local mucosal delivery may also be prepared to block sexual transmission.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens and liposomes targeted to macrophages containing, for example, phosphatidylserine) can also be used as pharmaceutically acceptable carriers. These can be prepared according to methods known to those skilled in the art, for example, as described in U.S. Pat. No. 4,522,811 and U.S. Pat. No. 5,643,599, the entire contents of which are incorporated herein.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease, disorder, or infection, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moieties to antibodies are well known, see, e.g., Arnon, et al., “Monoclonal Antibodies For Immunotargeting Of Drugs In Cancer Therapy”, in Monoclonal Antibodies And Cancer Therapy, Reisfeld, et al. (eds.), pp. 243-56 (Alan R. Liss, Inc. 1985); Hellstrom, et al., “Antibodies For Drug Delivery”, in Controlled Drug Delivery (2nd Ed.), Robinson, et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin, et al. (eds.), pp. 303-16 (Academic Press 1985), and Thorpe, et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen, et al. (1994) Proc. Natl. Acad. Sci., USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Methods of the Invention

The isolated nucleic acid molecules of the invention can be used, for example, to express SLIM polypeptides (e.g., via a recombinant expression vector in a host cell in gene therapy applications), to detect SLIM mRNA (e.g., in a biological sample) or a genetic alteration in a SLIM gene, and to modulate SLIM activity and/or expression, as described further below. The SLIM polypeptides can be used to treat or prevent a disorder that would benefit from the modulation of STAT expression and/or activity. In addition, the SLIM polypeptides can be used to screen for naturally occurring SLIM substrates, and to screen for drugs or compounds which modulate SLIM activity. Moreover, the anti-SLIM antibodies of the invention can be used to detect and isolate SLIM polypeptides, to regulate the bioavailability of SLIM polypeptides, and modulate SLIM activity.

A. Screening Assays

The invention provides methods (also referred to herein as “screening assays”) for identifying modulators, i.e., candidate or test compounds or agents (e.g., peptidomimetics, small molecules or other drugs) which modulate, for example one or more SLIM activity, e.g., the ability to 1) bind to STAT, 2) modulate STAT ubiquitination, 3) modulate STAT phosphorylation, 4) modulate IFN-γ production, 5) modulate STAT signaling, 6) modulate Th1 cell differentiation, 7) modulate protein folding, protein transport, and/or protein secretion, and 8) modulate protein degradation, or for testing or optimizing the activity of such agents.

The assays can be used to identify agents that modulate the function of SLIM and/or a SLIM-binding molecule, such as, but not limited to STAT, e.g., STAT4 and/or STAT1. For example, such agents may interact with SLIM or the SLIM-binding molecule (e.g., to inhibit or enhance their activity). The function of SLIM or the SLIM-binding molecule can be affected at any level, including transcription, protein expression, protein localization, and/or cellular activity. The subject assays can also be used to identify, e.g., agents that alter the interaction of SLIM or the SLIM-binding molecule with a binding partner, substrate, or cofactors, or modulate, e.g., increase, the stability of such interaction.

The subject screening assays can measure the activity of SLIM or a SLIM-binding protein directly (e.g., phosphorylation or ubiquitination), or can measure a downstream event controlled by modulation of SLIM or a SLIM-binding protein (e.g., IFNγ production, STAT signaling or Th1 cell differentiation).

The subject screening assays employ indicator compositions. These indicator compositions comprise the components required for performing an assay that detects and/or measures a particular event. The indicator compositions of the invention provide a reference readout and changes in the readout can be monitored in the presence of one or more test compounds. A difference in the readout in the presence and the absence of the compound indicates that the test compound is a modulator of the molecule(s) present in the indicator composition.

The indicator composition used in the screening assay can be a cell that expresses a SLIM polypeptide or a SLIM-binding molecule. For example, a cell that naturally expresses or, more preferably, a cell that has been engineered to express the protein by introducing into the cell an expression vector encoding the protein may be used. Preferably, the cell is a mammalian cell, e.g., a human cell. In one embodiment, the cell is a T cell. In another embodiment, the cell is a non-T cell. Alternatively, the indicator composition can be a cell-free composition that includes the protein (e.g., a cell extract or a composition that includes e.g., either purified natural or recombinant protein).

The indicator composition used in the screening assays of the invention can be a cell that expresses a SLIM family polypeptide or biologically active fragment thereof. For example, in one embodiment, the indicator composition comprises the amino acid sequence selected from the group consisting of SEQ ID NOs: 2, 5, 7, 9, 11, 13, 15, and 17.

Alternatively, the indicator compositions used in the screening assays of the invention comprise a polypeptide which comprises the consensus amino acid sequence set forth in SEQ ID NO:3.

In another embodiment, the indicator composition comprises more than one polypeptide. For example, in one embodiment the subject assays are performed in the presence of SLIM and/or at least one SLIM-binding molecule, such as, but nor limited to STAT, e.g., STAT4 and/or STAT1.

Compounds that modulate the expression and/or activity of SLIM, identified using the assays described herein can be useful for treating a subject that would benefit from the modulation of SLIM production.

In one embodiment, secondary assays can be used to confirm that the modulating agent affects the SLIM molecule in a specific manner. For example, compounds identified in a primary screening assay can be used in a secondary screening assay to determine whether the compound affects a SLIM-related activity. Accordingly, in another aspect, the invention pertains to a combination of two or more of the assays described herein. For example, a modulating agent can be identified using a cell-based or a cell-free assay, e.g., to detect binding, and the ability of the agent to modulate the activity of SLIM can be confirmed using a biological read-out to measure, e.g., cytokine production or Th1 cell differentiation, in vitro or in vivo.

Moreover, a modulator of SLIM expression and/or activity identified as described herein (e.g., a small molecule) may be used in an animal model to determine the efficacy, toxicity, or side effects of treatment with such a modulator. Alternatively, a modulator identified as described herein may be used in an animal model to determine the mechanism of action of such a modulator.

In one embodiment, the screening assays of the invention are high throughput or ultra high throughput (e.g., Fernandes, P. B., Curr Opin Chem Biol. 1998 2:597; Sundberg, S A, Curr Opin Biotechnol. 2000, 11:47).

Exemplary cell based and cell free assays of the invention are described in more detail below.

i. Cell Based Assays

The indicator compositions of the invention may be cells that express a SLIM or a SLIM-interacting molecule. For example, a cell that naturally expresses endogenous polypeptide, or, more preferably, a cell that has been engineered to express one or more exogenous polypeptides, e.g., by introducing into the cell an expression vector encoding the protein may be used in a cell based assay.

The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell (e.g., a murine cell, rhesus monkey, or a human cell). In a preferred embodiment, the cell is a human cell.

Preferably a cell line is used which expresses low levels of endogenous SLIM and/or a SLIM-interacting polypeptide and is then engineered to express recombinant protein.

Recombinant expression vectors that may be used for expression of polypeptides are known in the art. For example, the cDNA is first introduced into a recombinant expression vector using standard molecular biology techniques. A cDNA can be obtained, for example, by amplification using the polymerase chain reaction (PCR) or by screening an appropriate cDNA library.

When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufman, et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo, et al. (1982) Cell 29:99-108; Brinster, et al. (1982) Nature 296:39-42; Searle, et al. (1985) Mol. Cell. Biol. 5:1480-1489), heat shock (see e.g., Nouer, et al. (1991) in Heat Shock Response, e.d. Nouer, L., CRC, Boca Raton, Fla., pp167-220), hormones (see e.g., Lee, et al. (1981) Nature 294:228-232; Hynes, et al. (1981) Proc. Natl. Acad. Sci., USA 78:2038-2042; Klock, et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci., USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert, et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji, et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci., USA 86:5473-5477), pancreas-specific promoters (Edlund, et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Vector DNA may be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook, et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on a separate vector from that encoding SLIM or a SLIM-interacting polypeptide, on the same vector. Cells stably transfected with the introduced nucleic acid can be identified by drug selection (e.g., cells that have incorporated the selectable marker gene will survive, while the other cells die).

In one embodiment, within the expression vector coding sequences are operatively linked to regulatory sequences that allow for constitutive expression of the molecule in the indicator cell (e.g., viral regulatory sequences, such as a cytomegalovirus promoter/enhancer, may be used). Use of a recombinant expression vector that allows for constitutive expression of the genes in the indicator cell is preferred for identification of compounds that enhance or inhibit the activity of the molecule. In an alternative embodiment, within the expression vector the coding sequences are operatively linked to regulatory sequences of the endogenous gene (i.e., the promoter regulatory region derived from the endogenous gene). Use of a recombinant expression vector in which expression is controlled by the endogenous regulatory sequences is preferred for identification of compounds that enhance or inhibit the transcriptional expression of the molecule.

For example, an indicator cell can be transfected with an expression vector comprising a SLIM polypeptide, or biologically active fragment thereof, incubated in the presence and in the absence of a test compound, and the effect of the compound on the expression of the molecule or on a biological response regulated by SLIM, e.g., a SLIM-related activity, can be determined. The biological activities of SLIM include activities determined in vivo, or in vitro, according to standard techniques. Activity can be a direct activity, such as an association with or enzymatic activity, e.g., phosphorylation, e.g., tyrosine and serine phosphorylation, or ubiquitination, on a target molecule (e.g., STAT, e.g., STAT4 and/or STAT1). Alternatively, activity may be an indirect activity, such as, for example, a cellular signaling activity occurring downstream of the interaction of the protein with a target molecule or a biological effect occurring as a result of the signaling cascade triggered by that interaction, such as STAT signaling, IFNγ production or Th1 cell differentiation.

Compounds that modulate SLIM production, expression and/or activity of may be identified using various “read-outs.” For example, a variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art.

For example, in one embodiment, gene expression of SLIM, or a SLIM-binding molecule can be measured. In another embodiment, expression of a gene controlled by SLIM can be measured.

To determine whether a test compound modulates expression, in vitro transcriptional assays can be performed. For example, mRNA or protein expression can be measured using methods well known in the art. For instance, one or more of Northern blotting, slot blotting, ribonuclease protection, quantitative RT-PCR, or microarray analysis (e.g. Current Protocols in Molecular Biology (1994) Ausubel, F M et al., eds., John Wiley & Sons, Inc.; Freeman W M, et al., Biotechniques 1999 26:112; Kallioniemi, et al. 2001 Ann. Med. 33:142; Blohm and Guiseppi-Eli 2001 Curr Opin Biotechnol. 12:41) may be used to confirm that expression is modulated in cells treated with a modulating agent.

In another example, agents that modulate the expression of SLIM can be identified by operably linking the upstream regulatory sequences (e.g., the full length promoter and enhancer) of a SLIM to a reporter gene such as chloramphenicol acetyltransferase (CAT) or luciferase and introducing in into host cells. The ability of an agent to modulate the expression of the reporter gene product as compared to control cells (e.g., not exposed to the compound) can be measured.

As used interchangeably herein, the terms “operably linked” and “operatively linked” are intended to mean that the nucleotide sequence is linked to a regulatory sequence in a manner which allows expression of the nucleotide sequence in a host cell (or by a cell extract). Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described in Goeddel, Gene Expression Technology. Methods in Enzymology 185, Academic Press, San Diego, Calif. (1990). It should be understood that the design of the expression vector may depend on such factors as the choice of the host cell to be transfected and/or the type and/or amount of protein desired to be expressed.

In one embodiment, the level of expression of the reporter gene in the indicator cell in the presence of the test compound is higher than the level of expression of the reporter gene in the indicator cell in the absence of the test compound and the test compound is identified as a compound that stimulates the expression of a SLIM gene.

In another embodiment, the level of expression of the reporter gene in the indicator cell in the presence of the test compound is lower than the level of expression of the reporter gene in the indicator cell in the absence of the test compound and the test compound is identified as a compound that inhibits the expression of a SLIM gene.

In another embodiment, protein expression may be measured. For example, standard techniques such as Western blotting or in situ detection can be used.

In one embodiment, the ability of a compound to modulate IFN-γ production can be determined. Production of IFN-γ can be monitored, for example, using Northern or Western blotting. IFN-γ can also be detected using an ELISA assay or in a bioassay, e.g., employing cells which are responsive to IFN-γ (e.g., cells which proliferate in response to the cytokine or which survive in the presence of the cytokine) using standard techniques.

In one embodiment, the effect of a compound on a STAT signaling pathway can be determined. STAT phosphorylation/activation by JAK kinases leads to their translocation to the nucleus where they activate transcription of various cytokine genes by binding to specific DNA elements. Activated STATs are known to stimulate transcription of SOCS (suppressors of cytokine signaling) genes which bind phosphorylated JAKs and their receptors to prevent further phosphorylation of STATs and thus serve as a negative feedback regulatory loop. Other exemplary molecules in a STAT signaling pathway include but are not limited to, Ras, EGFR and PDGFR, and TGF-β (reviewed, in, for example, Rawlings, J. S. (2004) Journal of Cell Science 117:1281-1283). Accordingly, to determine the effect of a compound on a STAT signal transduction pathway, the ability of the compound to modulate the activation status of various molecules in the signal transduction pathway can be determined using standard techniques. In one embodiment, the expression of SOC is determined. In another embodiment, the phosphorylation of SOC is determined.

In one embodiment, modulation of the effect of the compound on the phosphorylation status of STAT, e.g., STAT1 and/or STAT4, by, for example, Western blotting, as described in the Examples herein, or by immunoblotting with antibodies specific to the phosphorylation status of STAT. In one embodiment, the compound modulates the effect of SLIM on the serine phosphorylation of STAT. In another embodiment, the compound modulates the effect of SLIM on the tyrosine phosphorylation of STAT.

In another embodiment, the effect of the compound on ubiquitination of, for example, STAT, can be measured, by, for example in vitro or in vivo ubiquitination assays. In vitro ubiquitination assays are described in, for example, Fuchs, S. Y., Bet al. (1997) J. Biol. Chem. 272:32163-32168. In vivo ubiquitination assays are described in, for example, Treier, M., L. et al. (1994) Cell 78:787-798.

In another embodiment, the effect of the compound on the degradation of, for example, a SLIM target molecule, such as a STAT, can be measured by, for example, coimmunoprecipitation of SLIM, or a biological fragment thereof, with, e.g., STAT, e.g., STAT4 and/or STAT1. Western blotting of the coimmunoprecipitate and probing of the blots with antibodies to SLIM and the SLIM target molecule can be quantitated to determine whether degradation has occurred.

In one embodiment, the ability of a compound to modulate protein folding or transport can be determined. The expression of a protein on the surface of a cell or the secretion of a secreted protein can be measured as indicators of protein folding and transport. Protein expression on a cell can be measured, e.g., using FACS analysis, surface iodination, immunoprecipitation from membrane preparations. Protein secretion can be measured, for example, by measuring the level of protein in a supernatant from cultured cells. The production of any secreted protein can be measured in this manner. The protein to be measured can be endogenous or exogenous to the cell. In preferred embodiment, the protein is selected from the group consisting of: α-fetoprotein, α1-antitrypsin, albumin, luciferase and immunoglobulins. The production of proteins can be measured using standard techniques in the art.

In one embodiment a downstream effect of modulation of SLIM production, e.g., the effect of a compound on Th1 cell differentiation, e.g., T cells, may be used as an indicator of modulation of SLIM or a SLIM-interacting protein. Th1 cell differentiation can be monitored directly (e.g. by microscopic examination of the cells), or indirectly, e.g., by monitoring one or more markers of Th1 cells (e.g., by FACs analysis and/or an increase in mRNA for a gene product associated with Th1 cells) or the expression of a cell surface marker. Standard methods for detecting mRNA of interest, such as reverse transcription-polymerase chain reaction (RT-PCR) and Northern blotting, are known in the art. Standard methods for detecting protein secretion in culture supernatants, such as enzyme linked immunosorbent assays (ELISA), are also known in the art. Proteins can also be detected using antibodies, e.g., in an immunoprecipitation reaction or for staining and FACS analysis.

The ability of the test compound to modulate SLIM or a SLIM-interacting polypeptide binding to a substrate or target molecule can also be determined. Determining the ability of the test compound to modulate, for example, SLIM, binding to a target molecule (e.g., a binding partner such as a substrate) can be accomplished, for example, by determining the ability of the molecules to be coimmunoprecipitated or by coupling the target molecule with a radioisotope or enzymatic label such that binding of the target molecule to SLIM or a SLIM-interacting polypeptide can be determined, e.g., by detecting the labeled SLIM target molecule in a complex. Alternatively, for example, SLIM, can be coupled with a radioisotope or enzymatic label to monitor the ability of a test compound to modulate, SLIM, binding to a target molecule in a complex.

Determining the ability of the test compound to bind to SLIM can be accomplished, for example, by coupling the compound with a radioisotope or enzymatic label such that binding of the compound can be determined by detecting the labeled compound in a complex. For example, targets can be labeled with ¹²⁵I, ³⁵S, ¹⁴C, or ³H, either directly or indirectly, and the radioisotope detected by direct counting of radioemmission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

In another embodiment, fluorescence technologies can be used, e.g., fluorescence polarization, time-resolved fluorescence, and fluorescence resonance energy transfer (Selvin, P R, Nat. Struct. Biol. 2000 7:730; Hertzberg R P and Pope A J, Curr Opin Chem Biol. 2000 4:445).

It is also within the scope of this invention to determine the ability of a compound to interact with SLIM, a SLIM-interacting molecule without the labeling of any of the interactants. For example, a microphysiometer may be used to detect the interaction of a compound with a SLIM, a SLIM-interacting molecule without the labeling of either the compound or the molecule (McConnell, H. M., et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate may be used as an indicator of the interaction between compounds.

In yet another aspect of the invention, the SLIM or a SLIM-interacting polypeptide protein or fragments thereof may be used as “bait protein” e.g., in a two-hybrid assay or three-hybrid assay (see, e.g., U.S. Pat. No. 5,283,317; Zervos, et al. (1993) Cell 72:223-232; Madura, et al. (11993) J. Biol. Chem. 268:12046-12054; Bartel, et al. (1993) Biotechniques 14:920-924; Iwabuchi, et al. (1993) Oncogene 8: 1693-1696; and Brent WO94/10300), to identify other proteins, which bind to or interact with SLIM or a SLIM-interacting polypeptide (“binding proteins” or “bp”) and are involved in SLIM or a SLIM-interacting molecule activity. Such SLIM- or SLIM-interacting molecule-binding proteins are also likely to be involved in the propagation of signals by the SLIM or a SLIM-interacting molecule proteins. The two-hybrid system is based on the modular nature of most transcription factors, which consist of separable DNA-binding and activation domains. Briefly, the assay utilizes two different DNA constructs. In one construct, the gene that codes for a SLIM or a SLIM-interacting molecule protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a SLIM- or a SLIM-interacting molecule-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the SLIM or a SLIM-interacting molecule protein.

ii. Cell-Free Assays

Alternatively, the indicator composition can be a cell-free composition that includes a SLIM and/or a SLIM-interacting molecule, e.g., a cell extract from a cell expressing the protein or a composition that includes purified either natural or recombinant protein.

In one embodiment, the indicator composition is a cell free composition. Polypeptides expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies may be used to produce a purified or semi-purified protein that may be used in a cell free composition. Alternatively, a lysate or an extract of cells expressing the protein of interest can be prepared for use as cell-free composition. Cell extracts with the appropriate post-translation modifications of proteins can be prepared using commercially available resources found at, for example Promega, Inc., and include but are not limited to reticulocyte lysate, wheat germ extract and E. coli S30 extract.

In one embodiment, compounds that specifically modulate an activity of SLIM or a SLIM-binding molecule may be identified. For example, compounds that modulate an activity of SLIM are identified based on their ability to modulate the interaction of SLIM with a target molecule to which SLIM binds. In another embodiment, compounds that modulate an activity of SLIM are identified based on their ability to modulate interaction of SLIM with a SLIM-binding molecule. Suitable assays are known in the art that allow for the detection of protein-protein interactions (e.g., immunoprecipitations and the like) or that allow for the detection of interactions between a DNA binding protein and a target DNA sequence (e.g., electrophoretic mobility shift assays, DNAse I footprinting assays and the like). By performing such assays in the presence and absence of test compounds, these assays may be used to identify compounds that modulate (e.g., inhibit or enhance) the interaction of SLIM or a SLIM-binding molecule with a target molecule.

In the methods of the invention for identifying test compounds that modulate an interaction between a SLIM-interacting protein and SLIM, the complete SLIM protein may be used in the method, or, alternatively, only portions of the protein may be used. For example, an isolated SLIM domain (e.g., a LIM domain and/or a PDZ domain), or a polypeptide comprising at least one of a LIM and/or PDZ domain, may be used. An assay may be used to identify test compounds that either stimulate or inhibit the interaction between the SLIM protein and a target molecule. A test compound that stimulates the interaction between the protein and a target molecule is identified based upon its ability to increase the degree of interaction between (e.g., SLIM and a target molecule) as compared to the degree of interaction in the absence of the test compound and such a compound would be expected to increase the activity of SLIM in the cell. A test compound that inhibits the interaction between the protein and a target molecule is identified based upon its ability to decrease the degree of interaction between the protein and a target molecule as compared to the degree of interaction in the absence of the compound and such a compound would be expected to decrease SLIM activity.

In one embodiment, the amount of binding of SLIM to a SLIM-interacting molecule in the presence of the test compound is greater than the amount of binding in the absence of the test compound, in which case the test compound is identified as a compound that enhances binding of SLIM to a SLIM interacting molecule In another embodiment, the amount of binding of the SLIM to the binding molecule in the presence of the test compound is less than the amount of binding of SLIM to the binding molecule in the absence of the test compound, in which case the test compound is identified as a compound that inhibits binding of SLIM to the binding molecule.

For example, binding of the test compound to SLIM or a SLIM-interacting polypeptide can be determined either directly or indirectly as described above. Determining the ability of SLIM protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo, et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) may be used as an indication of real-time reactions between biological molecules.

In another embodiment, the ability of a compound to modulate the ability of SLIM or a SLIM-interacting molecule to be acted on by an enzyme or to act on a substrate can be measured. In one embodiment, ubiquitination assays can be used to detect the ability of SLIMs to ubiquitinate a substrate, e.g., a STAT. Such assays are well-known in the art (see, for example, Klotzbucher, A., et al. (2002) Biol. Proceed. Online 4:62, incorporated herein by reference). In another embodiment, immunoblotting to determine the phosphorylation status of a SLIM target-molecule is used to detect the ability of SLIMs or a fragment thereof to phosphorylate a substrate.

In one embodiment of the above assay methods, it may be desirable to immobilize either SLIM or a SLIM-interacting polypeptide for example, to facilitate separation of complexed from uncomplexed forms of one or both of the proteins, or to accommodate automation of the assay. Binding to a surface can be accomplished in any vessel suitable for containing the reactants. Examples of Such vessels include microtitre plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or SLIM protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, proteins may be immobilized utilizing conjugation of biotin and streptavidin. Biotinylated protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with protein or target molecules but which do not interfere with binding of the protein to its target molecule can be derivatized to the wells of the plate, and unbound target or SLIM protein is trapped in the wells by antibody conjugation. Methods for detecting such complexes, in addition to those described above for the GST-immobilized complexes, include immunodetection of complexes using antibodies reactive with SLIM or a SLIM-interacting polypeptide or target molecule, as well as enzyme-linked assays which rely on detecting an enzymatic activity associated with the SLIM protein or binding molecule.

iii. Assays Using SLIM Deficient Cells

In another embodiment, the invention provides methods for identifying compounds that modulate a biological effect of SLIM using cells deficient in SLIM. As described in the Examples, inhibition of SLIM activity (e.g., by disruption of the SLIM gene) results in increased STAT, e.g., STAT4, production and enhanced IFNγ production by Th1 cells. Thus, cells deficient in SLIM can be used identify agents that modulate a biological response regulated by SLIM by means other than modulating SLIM itself (i.e., compounds that “rescue” the SLIM deficient phenotype). Alternatively, a “conditional knock-out” system, in which the SLIM gene is rendered non-functional in a conditional manner, can be used to create SLIM deficient cells for use in screening assays. For example, a tetracycline-regulated system for conditional disruption of a gene as described in WO 94/29442 and U.S. Pat. No. 5,650,298 can be used to create cells, or SLIM deficient animals from which cells can be isolated, that can be rendered SLIM deficient in a controlled manner through modulation of the tetracycline concentration in contact with the cells. For assays relating to other biological effects of SLIM a similar conditional disruption approach can be used or, alternatively, the RAG-2 deficient blastocyst complementation system can be used to generate mice with lymphoid organs that arise from embryonic stem cells with homozygous mutations of the SLIM gene. SLIM deficient lymphoid cells (e.g., thymic, splenic and/or lymph node cells) or purified SLIM deficient B cells from such animals can be used in screening assays.

In the screening method, cells deficient in SLIM are contacted with a test compound and a biological response regulated by SLIM is monitored. Modulation of the response in SLIM deficient cells (as compared to an appropriate control such as, for example, untreated cells or cells treated with a control agent) identifies a test compound as a modulator of the SLIM regulated response.

In one embodiment, the test compound is administered directly to a non-human SLIM deficient animal, preferably a mouse (e.g., a mouse in which the SLIM gene is conditionally disrupted by means described above, or a chimeric mouse in which the lymphoid organs are deficient in SLIM as described above), to identify a test compound that modulates the in vivo responses of cells deficient in SLIM. In another embodiment, cells deficient in SLIM are isolated from the non-human SLIM deficient animal, and contacted with the test compound ex vivo to identify a test compound that modulates a response regulated by SLIM in the cells deficient in SLIM.

Cells deficient in SLIM can be obtained from a non-human animals created to be deficient in SLIM. Preferred non-human animals include monkeys, dogs, cats, mice, rats, cows, horses, goats and sheep. In preferred embodiments, the SLIM deficient animal is a mouse. Mice deficient in SLIM can be made as described in the Examples. Non-human animals deficient in a particular gene product typically are created by homologous recombination. Briefly, a vector is prepared which contains at least a portion of the SLIM gene into which a deletion, addition or Substitution has been introduced to thereby alter, e.g., functionally disrupt, the endogenous SLIM gene. The SLIM gene preferably is a mouse SLIM gene. For example, a mouse SLIM gene can be isolated from a mouse genomic DNA library using the mouse SLIM cDNA as a probe. The mouse SLIM gene then can be used to construct a homologous recombination vector suitable for altering an endogenous SLIM gene in the mouse genome. In a preferred embodiment, the vector is designed such that, upon homologous recombination, the endogenous SLIM gene is functionally disrupted (i.e., no longer encodes a functional polypeptide; also referred to as a “knock out” vector).

Alternatively, the vector can be designed such that, upon homologous recombination, the endogenous SLIM gene is mutated or otherwise altered but still encodes functional polypeptide (e.g., the upstream regulatory region can be altered to thereby alter the expression of the endogenous SLIM polypeptide). In the homologous recombination vector, the altered portion of the SLIM gene is flanked at its 5′ and 3′ ends by additional nucleic acid of the SLIM gene to allow for homologous recombination to occur between the exogenous SLIM gene carried by the vector and an endogenous SLIM gene in an embryonic stem cell. The additional flanking SLIM nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced SLIM gene has homologously recombined with the endogenous SLIM gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology), 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, retroviral transduction of donor bone marrow cells from both wild type and SLIM null mice can be performed with the DN or dominant negative constructs to reconstitute irradiated RAG recipients. This will result in the production of mice whose lymphoid cells express only a dominant negative version of SLIM. B cells from these mice can then be tested for compounds that modulate a biological response regulated by SLIM.

In one embodiment of the screening assay, compounds tested for their ability to modulate a biological response regulated by SLIM are contacted with SLIM deficient cells by administering the test compound to a non-human SLIM deficient animal in vivo and evaluating the effect of the test compound on the response in the animal. The test compound can be administered to a non-human SLIM deficient animal as a pharmaceutical composition.

B. Test Compounds

A variety of test compounds can be evaluated using the screening assays described herein. The term “test compound” includes any reagent or test agent which is employed in the assays of the invention and assayed for its ability to influence the production, expression and/or activity of cytokines. More than one compound, e.g., a plurality of compounds, can be tested at the same time for their ability to modulate cytokine production, expression and/or activity in a screening assay. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening may be used to assay for the activity of a compound.

In certain embodiments, the compounds to be tested can be derived from libraries (i.e., are members of a library of compounds). While the use of libraries of peptides is well established in the art, new techniques have been developed which have allowed the production of mixtures of other compounds, Such as benzodiazepines (Bunin, et al. (1992). J. Am. Chem. Soc. 114:10987; DeWitt et al. (1993). Proc. Natl. Acad. Sci., USA 90:6909) peptoids (Zuckerman. (1994). J. Med. Chem. 37:2678) oligocarbamates (Cho, et al. (1993). Science. 261:11303), and hydantoins (DeWitt, et al. supra). An approach for the synthesis of molecular libraries of small organic molecules with a diversity of 104-105 as been described (Carell, et al. (1994). Angew. Chem. Int. Ed. Engl. 33:2059; Carell, et al. (1994) Angew. Chem. Int. Ed. Engl. 33:2061).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb, et al. (1994). Proc. Natl. Acad. Sci., USA 91:11422-; Horwell, et al. (1996) Immunopharmacology 33:68-; and in Gallop, et al. (1994); J. Med. Chem. 37:1233.

Exemplary compounds which can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S., et al. (1991) Nature 354:82-84; Houghten, R., et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z., et al. (1993) Cell 72:767-778); 3) antibodies (e.g., antibodies (e.g., intracellular, polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms of molecules (e.g., dominant negative mutant forms of SLIM or a SLIM-bindinig protein).

The test compounds of the present invention can be obtained using any of the numerous approaches in combinatorial library methods known in the art, including: biological libraries; spatially addressable parallel solid phase or Solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt, et al. (1993) Proc. Natl. Acad. Sci., U.S.A. 90:6909; Erb, et al. (1994) Proc. Natl. Acad. Sci., USA 91:11422; Zuckermann, et al. (1994) J. Med. Chem. 37:2678; Cho, et al. (1993) Science 261:1303; Carrell, et al. (1994) Angew. Chem. Int. Ed. Engl. 33:2059; Carell, et al. (1994) Angew. Chem. Int. Ed. Engl. 33:2061; and Gallop, et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull, et al. (1992) Proc. Natl. Acad. Sci., USA 89:1865-1869) or phage (Scott and Smith (1990) Science 249:386-390; Devlin (1990) Science 249:404-406; Cwirla, et al. (1990) Proc. Natl. Acad. Sci., USA 87:6378-6382; Felici (1991) J. Mol. Biol. 222:301-310; Ladner supra.).

Compounds identified in the subject screening assays may be used, e.g., in methods of modulating STAT signaling, IFNγ production, and/or Th1 cell differentiation. It will be understood that it may be desirable to formulate such compound(s) as pharmaceutical compositions (described supra) prior to contacting them with cells.

Once a test compound is identified that directly or indirectly modulates, e.g., production, expression and/or activity of a gene regulated by SLIM and/or a SLIM-binding molecule, by one of the variety of methods described herein, the selected test compound (or “compound of interest”) can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

The instant invention also pertains to compounds identified in the subject screening assays.

VI. Methods of Use

As described in the appended Examples, SLIM has a variety of biological effects on cells, including modulation of binding to STAT, modulation of STAT ubiquitination, modulation of STAT phosphorylation, modulation of IFN-γ production, modulation of STAT signaling, modulation of Th1 cell differentiation, modulation of protein folding, protein transport, and/or protein secretion, and/or modulation of protein degradation.

Accordingly, the subject methods employ agents that modulate a SLIM expression, processing, post-translational modification, or activity, or the expression, processing, post-translational modification, or activity of another molecule in a SLIM signaling pathway, e.g., STAT, IFN-γ, such that SLIM or the activity of a molecule in a SLIM signal transduction pathway is modulated. The subject methods are useful in both clinical and non-clinical settings.

In one embodiment, the instant methods can be performed in vitro. In another embodiment, the instant methods can be performed in a cell in vitro and then the treated cell can be administered to a subject.

The subject invention can also be used to treat various conditions or disorders that would benefit from modulation of the expression and/or activity of SLIM or a molecule in a SLIM signaling pathway, e.g., a STAT, IFN-γ. Exemplary disorders that would benefit from modulation of SLIM expression and/or activity are set forth herein. In one embodiment, the invention provides for modulation of a SLIM biological activity, e.g., IFN-γ production in vivo, by administering to the subject a therapeutically effective amount of a modulator of a SLIM such that a biological effect of SLIM in a subject is modulated. In another embodiment, the invention provides for modulation of a molecule in a SLIM signaling pathway, e.g., STAT, by administering to the subject a therapeutically effective amount of a modulator of a SLIM such that a biological effect of SLIM in a subject is modulated. For example, SLIM can be modulated to modulate IFN-γ production.

The term “subject” is intended to include living organisms in which an immune response can be elicited. Preferred subjects are mammals. Particularly preferred subjects are humans. Other examples of subjects include monkeys, dogs, cats, mice, rats, cows, horses, goats, sheep as well as other farm and companion animals. Modulation of SLIM expression and/or activity, in humans as well as veterinary applications, provides a means to regulate disorders arising from aberrant SLIM expression and/or activity in various disease states and is encompassed by the present invention.

Identification of compounds that modulate the biological effects of SLIM by directly or indirectly modulating SLIM expression and/or activity allows for selective manipulation of these biological effects in a variety of clinical situations using the modulatory methods of the invention. For example, the stimulatory methods of the invention (i.e., methods that use a stimulatory agent) can result in increased expression and/or activity of SLIM, which inhibits, e.g., STAT expression and/or activity, and can reduce cytokine production, e.g., IFN-γ production, and reduce Th1 cell differentiation. In contrast, the inhibitory methods of the invention (i.e., methods that use an agent that inhibits SLIM) can have the opposite effects.

Application of the modulatory methods of the invention to the treatment of a disorder may result in cure of the disorder, a decrease in the type or number of symptoms associated with the disorder, either in the long term or short term (i.e., amelioration of the condition) or simply a transient beneficial effect to the subject.

Application of the immunomodulatory methods of the invention is described in further detail below.

i. Exemplary Inhibitory Compounds

According to a modulatory method of the invention, the expression and/or activity of a SLIM family member, or biological fragment thereof, e.g., the LIM domain, is inhibited in a cell by contacting the cell with an inhibitory agent. An inhibitory agent of the invention can be, for example, an antisense nucleic acid molecule that is complementary to a gene encoding a SLIM polypeptide or to a portion of said gene, or a recombinant expression vector encoding said antisense nucleic acid molecule. The use of antisense nucleic acids to downregulate the expression of a particular polypeptide in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid of the invention is a compound that mediates RNAi. RNA interfering agents include, but are not limited to, nucleic acid molecules including RNA molecules which are homologous to the target gene or genomic sequence, e.g., a SLIM family member, or a fragment thereof, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genies Dell 13, 3191-3197(1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion.

Exemplary siRNA molecules specific for murine SLIM are shown below:

Beginning at position 158 of SEQ ID NO:1:

| | |
|---|---|
| Sense strand siRNA: | |
| GGUCACAGAGCGGGGCAAGtt | (SEQ ID NO:25) |
| Antisense strand siRNA: | |
| CUUGCCCCGCUCUGUGACCtt | (SEQ ID NO:26) |

Beginning at position 1016 of SEQ ID NO:1:

| | |
|---|---|
| Sense strand siRNA: | |
| GAUGCGCGGCCACUUCUGGtt | (SEQ ID NO:27) |
| Antisense strand siRNA: | |
| CCAGAAGUGGCCGCGCAUCtt | (SEQ ID NO:28) |

Exemplary siRNA molecules specific for rat SLIM are shown below:

Beginning at position 135 of SEQ ID NO:4:

| | |
|---|---|
| Sense strand siRNA: | |
| UGUGGUGGGACCAGCACCUtt | (SEQ ID NO:29) |
| Antisense strand siRNA: | |
| AGGUGCUGGUCCCACCACAtt | (SEQ ID NO:30) |

Beginning at position 1065 of SEQ ID NO:4:

| | |
|---|---|
| Sense strand siRNA: | |
| CCUGAAGAUGCGGGGUCACtt | (SEQ ID NO:31) |
| Antisense strand siRNA: | |
| GUGACCCCGCAUCUUCAGGtt | (SEQ ID NO:32) |

Exemplary siRNA molecules specific for human SLIM are shown below:

Beginning at position 491 of SEQ ID NO:8:

| | |
|---|---|
| Sense strand siRNA: | |
| GGUGGCCGAGCGGGGCAAAtt | (SEQ ID NO:33) |
| Antisense strand siRNA: | |
| UUUGCCCCGCUCGGCCACCtt | (SEQ ID NO:34) |

Beginning at position 1406 of SEQ ID NO:8:

| | |
|---|---|
| Sense strand siRNA: | |
| GCAUGCCCGCCAGCGCUACtt | (SEQ ID NO:35) |
| Antisense strand siRNA: | |
| GUAGCGCUGGCGGGCAUGCtt | (SEQ ID NO:36) |

In one embodiment one or more of the chemistries described above for use in antisense RNA can be employed.

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g. hammerhead ribozymes (described in Haselhoff and Gerlach, 1988, Nature 334:585-591) may be used to catalytically cleave SLIM mRNA transcripts to thereby inhibit translation of NIP45 mRNA. A ribozyme having specificity SLIM-encoding nucleic acid can be designed, e.g., based upon the nucleotide sequence of SEQ ID NO:1 or another nucleic acid molecule encoding another SLIM polypeptide. For example, a derivative of a Tetrahymena L-19 IVS RNA can be constructed in which the nucleotide sequence of the active site is complementary to the nucleotide sequence to be cleaved in a SLIM-encoding mRNA. See, e.g., Cech et al. U.S. Pat. No. 4,987,071; and Cech et al. U.S. Pat. No. 5,116,742. Alternatively, NIP45 mRNA may be used to select a catalytic RNA having a specific ribonuclease activity from a pool of RNA molecules. See, e.g., Bartel, D. and Szostak, J. W., 1993, Science 261:1411-1418.

Alternatively, gene expression can be inhibited by targeting nucleotide sequences complementary to the regulatory region of a SLIM (e.g., the SLIM promoter and/or enhancers) to form triple helical structures that prevent transcription of the a SLIM gene in target cells. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12):807-15.

In yet another embodiment, the SLIM nucleic acid molecules of the present invention can be modified at the base moiety, sugar moiety or phosphate backbone to improve, e.g. the stability, hybridization, or solubility of the molecule. For example, the deoxyribosc phosphate backbone of the nucleic acid molecules can be modified to generate peptide nucleic acids (see Hyrup B. et al., 1996, Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al., 1996, supra; Perry-O'Keefe et al., 1996, Proc. Natl. Acad. Sci. USA 93: 14670-675.

PNAs of a SLIM nucleic acid molecules may be used in therapeutic and diagnostic applications. For example, PNAs may be used as antisense or antigene agents for sequence-specific modulation of gene expression by, for example, inducing transcription or translation arrest or inhibiting replication. PNAs of a SLIM nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B., 1996, supra)); or as probes or primers for DNA sequencing or hybridization (Hyrup B. et al., 1996, supra; Perry-O'Keefe supra).

In another embodiment, PNAs of a SLIM can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of a SLIM nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B., 1996, supra). The synthesis of PNA-DNA chimeras can be performed as described in Hyrup B., 1996, supra and Finn P. J. et al., 1996, Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, may be used as a between the PNA and the 5′ end of DNA (Mag, M. et al., 1989, Nucleic Acid Res. 17: 5973-88). PNA monomers are then coupled in a stepwise manner to produce a chimeric molecule with a 5′ PNA segment and a 3′ DNA segment (Finn P. J. et al., 1996, supra). Alternatively, chimeric molecules can be synthesized with a 5′ DNA segment and a 3′ PNA segment (Peterser, K. H. et al., 1975, Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. US. 86:6553-6556; Lemaitre et al., 1987, Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al., 1988, Bio-Techniques 6:958-976) or intercalating agents. (See, e.g., Zon, 1988, Pharm. Res. 5:539-549). To this end, the oligonucleotide may be conjugated to another molecule, (e.g., a peptide, hybridization triggered cross-linking agent, transport agent, or hybridization-triggered cleavage agent).

Antisense polynucleotides may be produced from a heterologous expression cassette in a transfectant cell or transgenic cell. Alternatively, the antisense polynucleotides may comprise soluble oligonucleotides that are administered to the external milieu, either in the culture medium in vitro or in the circulatory system or in interstitial fluid in vivo. Soluble antisense polynucleotides present in the external milieu have been shown to gain access to the cytoplasm and inhibit translation of specific mRNA species.

In another embodiment, an inhibitory agent of the invention is a small molecule which interacts with a SLIM family member protein to thereby inhibit the activity of the SLIM family member. Small molecule inhibitors of a SLIM family member can be identified using database searching programs capable of scanning a database of small molecules of known three-dimensional structure for candidates which fit into the target protein site known in the art. Suitable software programs include, for example, CATALYST (Molecular Simulations Inc., San Diego, Calif.), UNITY (Tripos Inc., St Louis, Mo.), FLEXX (Rarey et al., J. Mol. Biol. 261: 470-489 (1996)), CHEM-3 DBS (Oxford Molecular Group, Oxford, UK), DOCK (Kuntz et al., J. Mol. Biol 161: 269-288 (1982)), and MACCS-3D (MDL Information Systems Inc., San Leandro, Calif.). The molecules found in the search may not necessarily be leads themselves, however, such candidates might act as the framework for further design, providing molecular skeletons to which appropriate atomic replacements can be made. The scaffold, functional groups, linkers and/or monomers may be changed to maximize the electrostatic, hydrogen bonding, and hydrophobic interactions with the target protein. Goodford (Goodford J Med Chem 28:849-857 (1985)) has produced a computer program, GRID, which seeks to determine regions of high affinity for different chemical groups (termed probes) on the molecular surface of the binding site. GRID hence provides a tool for suggesting modifications to known ligands that might enhance binding. A range of factors, including electrostatic interactions, hydrogen bonding, hydrophobic interactions, desolvation effects, conformational strain or mobility, chelation and cooperative interaction and motions of ligand and enzyme, all influence the binding effect and should be taken into account in attempts to design small molecule inhibitors.

Small molecule inhibitors of a SLIM family member can also be identified using computer-assisted molecular design methods comprising searching for fragments which fit into a binding region subsite and link to a predefined scaffold can be used. The scaffold itself may be identified in such a manner. Programs suitable for the searching of such functional groups and monomers include LUDI (Boehm, J Comp. Aid. Mol. Des. 6:61-78 (1992)), CAVEAT (Bartlett et al. in “Molecular Recognition in Chemical and Biological Problems”, special publication of The Royal Chem. Soc., 78:182-196 (1989)) and MCSS (Miranker et al. Proteins 11: 29-34 (1991)).

Yet another computer-assisted molecular design method for identifying small molecule inhibitors of a SLIM family member protein comprises the de novo synthesis of potential inhibitors by algorithmic connection of small molecular fragments that will exhibit the desired structural and electrostatic complementarity with the active binding site of the SLIM protein. The methodology employs a large template set of small molecules with are iteratively pieced together in a model of the SLIM binding site. Programs suitable for this task include GROW (Moon et al. Proteins 11:314-328 (1991)) and SPROUT (Gillet et al. J Comp. Aid. Mol. Des. 7:127 (1993)).

The suitability of small molecule inhibitor candidates can be determined using an empirical scoring function, which can rank the binding affinities for a set of inhibitors. For an example of such a method see Muegge et al. and references therein (Muegge et al., J Med. Chem. 42:791-804 (1999)). Other modeling techniques can be used in accordance with this invention, for example, those described by Cohen et al. (J. Med. Chem. 33: 883-894 (1994)); Navia et al. (Current Opinions in Structural Biology; 2: 202-210 (1992)); Baldwin et al. (J. Med. Chem. 32: 2510-2513 (1989)); Appelt et al. (J. Med. Chem. 34: 1925-1934 (1991)); and Ealick et al. (Proc. Nat. Acad. Sci. USA 88: 11540-11544 (1991)).

Yet another form of an inhibitory agent of the invention is an inhibitory form of human SLIM, also referred to herein as a dominant negative inhibitor. Many proteins are known to homodimerize and to heterodimerize. One means to inhibit the activity of molecules that form dimers is through the use of a dominant negative inhibitor that has the ability to dimerize with a functional molecule but that lacks the ability to perform its normal biological activity (see e.g., Petrak, D. et al. (1994) J. Immunol. 153:2046-2051). By dimerizing with SLIM, such dominant negative inhibitors can inhibit their functional activity.

Accordingly, an inhibitory agent of the invention can be a form of a SLIM polypeptide that has the ability to dimerize with other proteins but that lacks the ability to perform its normal biological activity, such as phosphorylation of STAT. This dominant negative form of a SLIM polypeptide may be, for example, a mutated form of SLIM in which the LIM and/or PDZ domain has been removed. Such dominant negative human SLIM proteins can be expressed in cells using a recombinant expression vector encoding the SLIM polypeptide, which is introduced into the cell by standard transfection methods. To express a mutant form of SLIM lacking a LIM and/or PDZ domain, nucleotide sequences encoding the corresponding domains of SLIM are removed from the SLIM coding sequences by standard recombinant DNA techniques. The truncated DNA is inserted into a recombinant expression vector, which is then introduced into a cell to allow for expression of the truncated SLIM, lacking a LIM and/or PDZ domain, in the cell.

Other inhibitory agents that can be used to inhibit the expression and/or activity of a SLIM family member polypeptide include chemical compounds that directly inhibit a SLIM family member or compounds that inhibit the interaction between a SLIM family member and target DNA or another polypeptide. Such compounds can be identified using screening assays that select for such compounds, as described in detail above.

B. Exemplary Stimulatory Agents

According to a modulatory method of the invention, the expression and/or activity of a SLIM family member is stimulated in a cell by contacting the cell with a stimulatory agent. Examples of such stimulatory agents include active SLIM family member polypeptides, or biologically active fragments thereof, and nucleic acid molecules encoding SLIM family members, or biologically active fragments thereof, that are introduced into the cell to increase SLIM family member expression and/or activity in the cell. A preferred stimulatory agent is a nucleic acid molecule encoding a SLIM family member polypeptide, or biologically active fragments thereof, wherein the nucleic acid molecule is introduced into the cell in a form suitable for expression of the active SLIM family member polypeptide, or biologically active fragment thereof, in the cell. To express a SLIM polypeptide in a cell, typically a SLIM-encoding DNA, or DNA encoding a biologically active fragment of SLIM, is first introduced into a recombinant expression vector using standard molecular biology techniques, as described herein. A SLIM-encoding DNA can be obtained, for example, by amplification using the polymerase chain reaction (PCR), using primers based on the SLIM nucleotide sequence. Following isolation or amplification of SLIM-encoding DNA, the DNA fragment is introduced into an expression vector and transfected into target cells by standard methods, as described herein.

Other stimulatory agents that can be used to stimulate the activity of a SLIM polypeptide are chemical compounds that stimulate SLIM activity in cells, such as compounds that directly stimulate SLIM polypeptide and compounds that promote the interaction between SLIM and target DNA or other polypeptides. Such compounds can be identified using screening assays that select for such compounds, as described in detail above.

The modulatory methods of the invention can be performed in vitro (e.g., by culturing the cell with the agent or by introducing the agent into cells in culture) or, alternatively, in vivo (e.g., by administering the agent to a subject or by introducing the agent into cells of a subject, such as by gene therapy). For practicing the modulatory method in vitro, cells can be obtained from a subject by standard methods and incubated (i.e., cultured) in vivo with a modulatory agent of the invention to modulate SLIM expression and/or activity in the cells. For example, peripheral blood mononuclear cells (PBMCs) can be obtained from a subject and isolated by density gradient centrifugation, e.g., With Ficoll/Hypaque. Specific cell populations can be depleted or enriched using standard methods. For example, T cells can be enriched for example, by positive selection using antibodies to T cell surface markers, for example by incubating cells with a specific primary monoclonal antibody (mAb), followed by isolation of cells that bind the mAb using magnetic beads coated with a secondary antibody that binds the primary mAb. Specific cell populations can also be isolated by fluorescence activated cell sorting according to standard methods. If desired, cells treated in vitro with a modulatory agent of the invention can be readministered to the subject. For administration to a subject, it may be preferable to first remove residual agents in the culture from the cells before administering them to the subject. This can be done for example by a Ficoll/Hypaque gradient centrifugation of the cells. For further discussion of ex vivo genetic modification of cells followed by readministration to a subject, see also U.S. Pat. No. 5,399,346 by W. F. Anderson et al.

For stimulatory or inhibitory agents that comprise nucleic acids (including recombinant expression vectors encoding SLIM polypeptide, antisense RNA, or dominant negative inhibitors), the agents can be introduced into cells of the subject using methods known in the art for introducing nucleic acid (e.g., DNA) into cells in vivo. Examples of such methods encompass both non-viral and viral methods, including:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Cationic Lipids: Naked DNA can be introduced into cells in vivo by complexing the DNA with cationic lipids or encapsulating the DNA in cationic liposomes. Examples of suitable cationic lipid formulations include N-[-1-(2,3-dioleoyloxy)propyl]N,N,N-triethylammonium chloride (DOTMA) and a 1:1 molar ratio of 1,2-dimyristyloxy-propyl-3-dimetlhylhydroxyethylammonium bromide (DMRIE) and dioleoyl phosphatidylethanolamine (DOPE) (see e.g. Logan. J. J. et al. (1995) Gene Therapy 2:38-49; San, H. et al. (1993) Human Gene Therapy 4:781-788).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263: 14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper-virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

In a preferred embodiment, a retroviral expression vector encoding SLIM is used to express SLIM polypeptide in cells in vivo, to thereby stimulate SLIM polypeptide activity in vivo. Such retroviral vectors can be prepared according to standard methods known in the art (discussed further above).

A modulatory agent, such as a chemical compound, can be administered to a subject as a pharmaceutical composition. Such compositions typically comprise the modulatory agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described above in subsection IV.

The identification of SLIM as a key regulator of the development of Th1 cells described herein, and in the repression of the Th2 phenotype, allows for selective manipulation of T cell subsets in a variety of clinical situations using the modulatory methods of the invention. The stimulatory methods of the invention (i.e., methods that use a stimulatory agent to enhance SLIM expression and/or activity) result in production of IFN-γ, with concomitant promotion of a Th1 response and downregulation of both IL-2 and IL-4, thus downmodulating the Th2 response. In contrast, the inhibitory methods of the invention (i.e., methods that use an inhibitory agent to downmodulate SLIM expression and/or activity) inhibit the production of IFN-γ, with concomitant downregulation of a Th1 response and promotion of a Th2 response. Thus, to treat a disease condition wherein a Th1 response is beneficial, a stimulatory method of the invention is selected such that Th1 responses are promoted while downregulating Th2 responses. Alternatively, to treat a disease condition wherein a Th2 response is beneficial, an inhibitory method of the invention is selected such that Th1 responses are downregulated while promoting Th2 responses. Application of the methods of the invention to the treatment of diseases or conditions may result in cure of the condition, a decrease in the type or number of symptoms associated with the condition, either in the long term or short term (i.e., amelioration of the condition) or simply a transient beneficial effect to the subject.

Numerous diseases or conditions associated with a predominant Th1 or Th2-type response have been identified and would benefit from modulation of the type of response mounted in the individual suffering from the disease condition. Application of the immunomodulatory methods of the invention to such diseases or conditions is described in further detail below.

A. Allergies

Allergies are mediated through IgE antibodies whose production is regulated by the activity of Th2 cells and the cytokines produced thereby. In allergic reactions, IL-4 is produced by Th2 cells, which further stimulates production of IgE antibodies and activation of cells that mediate allergic reactions, i.e., mast cells and basophils. IL-4 also plays an important role in eosinophil mediated inflammatory reactions. Accordingly, the stimulatory methods of the invention can be used to inhibit the production of Th2-associated cytokines, and in particular IL-4, in allergic patients as a means to downregulate production of pathogenic IgE antibodies. A stimulatory agent may be directly administered to the subject or cells (e.g., Thp cells or Th2 cells) may be obtained from the subject, contacted with a stimulatory agent ex vivo, and readministered to the subject. Moreover, in certain situations it may be beneficial to coadminister to the subject the allergen together with the stimulatory agent or cells treated with the stimulatory agent to inhibit (e.g., desensitize) the allergen-specific response. The treatment may be further enhanced by administering other Th1-promoting agents, such as the cytokine IL-12 or antibodies to Th2-associated cytokines (e.g., anti-IL-4 antibodies), to the allergic subject in amounts sufficient to further stimulate a Th1-type response.

B. Cancer

The expression of Th2-promoting cytokines has been reported to be elevated in cancer patients (see e.g., Yamamura, M., et al. (1993) J. Clin. Invest. 91:1005-1010; Pisa, P., et al. (1992) Proc. Natl. Acad. Sci. USA 89:7708-7712) and malignant disease is often associated with a shift from Th1 type responses to Th2 type responses along with a worsening of the course of the disease. Accordingly, the stimulatory methods of the invention can be used to inhibit the production of Th2-associated cytokines in cancer patients, as a means to counteract the Th1 to Th2 shift and thereby promote an ongoing Th1 response in the patients to ameliorate the course of the disease. The stimulatory method can involve either direct administration of an stimulatory agent to a subject with cancer or ex vivo treatment of cells obtained from the subject (e.g., Thp or Th2 cells) with a stimulatory agent followed by readministration of the cells to the subject. The treatment may be further enhanced by administering other Th1-promoting agents, such as the cytokine IL-12 or antibodies to Th2-associated cytokines (e.g., anti-IL-4 antibodies), to the recipient in amounts sufficient to further stimulate a Th 1-type response.

C. Infectious Diseases

The expression of Th2-promoting cytokines also has been reported to in crease during a variety of infectious diseases, including HIV infection, tuberculosis, leishmaniasis, schistosomiasis, filarial nematode infection and intestinal nematode infection (see e.g.; Shearer, G. M. an d Clerici, M. (1992) Prog. Chem. Immunol. 54:21-43; Clerici, M and Shearer, G. M. (1993) Immunology Today 14:107-111; Fauci, A. S. (1988) Science 239:617-623; Locksley, R. M. and Scott, P. (1992) Immunoparasitolgy Today 1:A58-A61; Pearce, E. J., et al. (1991) J. Exp. Med. 173:159-166; Grzych, J-M., et al. (1991) J. Immunol. 141:1322-1327; Kullberg, M. C., et al. (1992) J. Immunol. 148:3264-3270; Bancroft, A. J., et al. (1993) J. Immunol. 150:1395-1402; Pearlman, E., et al. (1993) Infect. Immun. 61:1105-1112; Else, K. J., et al. (1994) J. Exp. Med. 179:347-351) and such infectious diseases are also associated with a Th1 to Th2 shift in the immune response. Accordingly, the stimulatory methods of the invention can be used to inhibit the production of Th2-associated cytokines in subjects with infectious diseases, as a means to counteract the Th1 to Th2 shift and thereby promote an ongoing Th1 response in the patients to ameliorate the course of the infection. The stimulatory method can involve either direct administration of an inhibitory agent to a subject with an infectious disease or ex vivo treatment of cells obtained from the subject (e.g., Thp or Th2 cells) with a stimulatory agent followed by readministration of the cells to the subject. The treatment may be further enhanced by administering other Th1-promoting agents, such as the cytokine IL-12 or antibodies to Th2-associated cytokines (e.g., anti-IL-4 antibodies), to the recipient in amounts sufficient to further stimulate a Th1-type response.

D. Autoimmune Diseases

The inhibitory methods of the invention can be used therapeutically in the treatment of autoimmune diseases that are associated with a Th2-type dysfunction. Many autoimmune disorders are the result of inappropriate activation of T cells that are reactive against self tissue and that promote the production of cytokines and autoantibodies involved in the pathology of the diseases. Modulation of T helper-type responses can have an effect on the course of the autoimmune disease. For example, in experimental allergic encephalomyelitis (EAE), stimulation of a Th2-type response by administration of IL-4 at the time of the induction of the disease diminishes the intensity of the autoimmune disease (Paul, W. E., et al. (1994) Cell 76:241-251). Furthermore, recovery of the animals from the disease has been shown to be associated with an increase in a Th2-type response as evidenced by an increase of Th2-specific cytokines (Koury, S. J., et al. (1992) J. Exp. Med. 176:1355-1364). Moreover, T cells that can suppress EAE secrete Th2-specific cytokines (Chen, C., et al. (1994) Immunity 1:147-154). Since stimulation of a Th2-type response in EAE has a protective effect against the disease, stimulation of a Th2 response in subjects with multiple sclerosis (for which EAE is a model) is likely to be beneficial therapeutically. The inhibitory methods of the invention can be used to effect such a decrease.

Similarly, stimulation of a Th2-type response in type I diabetes in mice provides a protective effect against the disease. Indeed, treatment of NOD mice with IL-4 (which promotes a Th2 response) prevents or delays onset of type I diabetes that normally develops in these mice (Rapoport, M. J., et al. (1993) J. Exp. Med. 178:87-99). Thus, stimulation of a Th2 response in a subject suffering from or susceptible to diabetes may ameliorate the effects of the disease or inhibit the onset of the disease.

Yet another autoimmune disease in which stimulation of a Th2-type response may be beneficial is rheumatoid arthritis (RA). Studies have shown that patients with rheumatoid arthritis have predominantly Th1 cells in synovial tissue (Simon, A. K., et al. (1994) Proc. Natl. Acad. Sci. USA 91:8562-8566). By stimulating a Th2 response in a subject with RA, the detrimental Th1 response can be concomitantly downmodulated to thereby ameliorate the effects of the disease.

Accordingly, the inhibitory methods of the invention can be used to stimulate production of Th2-associated cytokines in subjects suffering from, or susceptible to, an autoimmune disease in which a Th2-type response is beneficial to the course of the disease. The inhibitory method can involve either direct administration of an inhibitory agent to the subject or ex vivo treatment of cells obtained from the subject (e.g., Thp, Th1 cells, B cells, non-lymphoid cells) with an inhibitory agent followed by readministration of the cells to the subject. The treatment may be further enhanced by administering other Th2-promoting agents, such as IL-4 itself or antibodies to Th1-associated cytokines, to the subject in amounts sufficient to further stimulate a Th2-type response.

In contrast to the autoimmune diseases described above in which a Th2 response is desirable, other autoimmune diseases may be ameliorated by a Th1-type response. Such diseases can be treated using a stimulatory agent of the invention (as described above for cancer and infectious diseases). The treatment may be further enhanced by administrating a Th 1-promoting cytokine (e.g., IFN-γ) to the subject in amounts sufficient to further stimulate a Th1-type response.

The efficacy of agents for treating autoimmune diseases can be tested in the above described animal models of human diseases (e.g., EAE as a model of multiple sclerosis and the NOD mice as a model for diabetes) or other well characterized animal models of human autoimmune diseases. Such animal models include the mrl/lpr/lpr mouse as a model for lupus erythematosus, murine collagen-induced arthritis as a model for rheumatoid arthritis, and murine experimental myasthenia gravis (see Paul ed., Fundatmental Immunology, Raven Press, New York, 1989, pp. 840-856). A modulatory (i.e., stimulatory or inhibitory) agent of the invention is administered to test animals and the course of the disease in the test animals is then monitored by the standard methods for the particular model being used. Effectiveness of the modulatory agent is evidenced by amelioration of the disease condition in animals treated with the agent as compared to untreated animals (or animals treated with a control agent).

Non-limiting examples of autoimmune diseases, disorders and conditions having an autoimmune component that may be treated according to the invention include diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia greata, allergic responses due to arthropod bite reactions, Crohn's disease, aphthous ulcer, iritis, conjunctivitis, keratoconjunctivitis, ulcerative colitis, allergic asthma, cutaneous lupus erythematosus, scleroderma, vaginitis, proctitis, compound eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

In a particular embodiment, diseases, disorders and conditions that may be treated by the methods of the invention include Crohn's disease and ulcerative colitis, which are the two major forms of inflammatory bowel diseases (IBD) in humans. Cytokines produced by T lymphocytes appear to initiate and perpetuate chronic intestinal inflammation. Crohn's disease is associated with increased production of T helper 1 (Th1) type cytokines such as IFN-γ and TNF. Ulcerative colitis is generally associated with T cells producing large amounts of the Th2 type cytokines and is referred to herein as “Th2-mediated colitis.” “Th1-mediated colitis” refers to a Crohn's disease profile as well as to the Th1 type response which can occur in ulcerative colitis. In Th 1-mediated colitis, agents which inhibit the activity of SLIM provide a protective effect. In Th2-mediated colitis, agents which stimulate the activity of SLIM provide a protective effect.

In another particular embodiment, diseases, disorders and conditions that may be treated by the methods of the invention include asthma, which is a disease of the bronchial tubes, or airways of the lungs, characterized by tightening of these airways. Production of IL-4, IL-5 and IL-13 has been associated with the development of an asthma-like phenotype. Accordingly, agents of the invention which stimulate the activity of SLIM provide a protective effect against asthma.

E. Transplantation

While graft rejection or graft acceptance may not be attributable exclusively to the action of a particular T cell subset (i.e., Th1 or Th2 cells) in the graft recipient (for a discussion see Dallman, M. J. (1995) Curr. Opin. Immunol. 7:632-638), numerous studies have implicated a predominant Th2 response in prolonged graft survival or a predominant Th1 response in graft rejection. For example, graft acceptance has been associated with production of a Th2 cytokine pattern and/or graft rejection has been associated with production of a Th1 cytokine pattern (see e.g., Takeuchi, T. et al. (1992) Transplantation 53:1281-1291; Tzakis, A. G. et al. (1994) J. Pediatr. Surg. 29:754-756; Thai, N. L. et al. (1995) Transplantation 59:274-281). Additionally, adoptive transfer of cells having a Th2 cytokine phenotype prolongs skin graft survival (Maeda, H. et al. (1994) Int. Immunol. 6:855-862) and reduces graft-versus-host disease (Fowler, D. H. et al. (1994) Blood 84:3540-3549; Fowler, D. H. et al. (1994) Prog. Clin. Biol. Res. 389:533-540). Still further, administration of IL-4, which promotes Th2 differentiation, prolongs cardiac allograft survival (Levy, A. E. and Alexander, J. W. (1995) Transplantation 60:405-406), whereas administration of IL-12 in combination with anti-IL-10 antibodies, which promotes Th1 differentiation, enhances skin allograft rejection (Gorczynski, R. M. et al. (1995) Transplantation 60:1337-1341).

Accordingly, the inhibitory methods of the invention can be used to stimulate production of Th2-associated cytokines in transplant recipients to prolong Survival of the graft. The inhibitory methods can be used both in solid organ transplantation and in bone marrow transplantation (e.g., to inhibit graft-versus-host disease). The inhibitory method can involve either direct administration of an inhibitory agent to the transplant recipient or ex-vivo treatment of cells obtained from the subject (e.g., Thp, Th1 cells, B cells, non-lymphoid cells) with an inhibitory agent followed by readministration of the cells to the subject. The treatment may be further enhanced by administering other Th2-promoting agents, such as IL-4 itself or antibodies to Th1-associated cytokines, to the recipient in amounts sufficient to further inhibit a Th2-type response.

In addition to the foregoing disease situations, the modulatory methods of the invention also are useful for other purposes. For example, the stimulatory methods of the invention (i.e., methods using a stimulatory agent) can be used to stimulate production of Th1-promoting cytokines (e.g., interferon-γ) in vitro for commercial production of these cytokines (e.g., cells can be contacted with the stimulatory agent in vitro to stimulate interferon-γ production and the interferon-γ can be recovered from the culture supernatant, further purified if necessary, and packaged for commercial use).

Furthermore, the modulatory methods of the invention can be applied to vaccinations to promote either a Th1 or a Th2 response to an antigen of interest in a subject. That is, the agents of the invention can serve as adjuvants to direct an immune response to a vaccine either to a Th1 response or a Th2 response. For example, to promote an antibody response to an antigen of interest (i.e., for vaccination purposes), the antigen and an inhibitory agent of the invention can be coadministered to a subject to promote a Th2 response to the antigen in the subject, since Th2 responses provide efficient B cell help and promote IgG1 production. Alternatively, to promote a cellular immune response to an antigen of interest, the antigen and a stimulatory agent of the invention can be coadministered to a subject to promote a Th1 response to the antigen in a subject, since Th1 responses favor the development of cell-mediated immune responses (e.g., delayed hypersensitivity responses). The antigen of interest and the modulatory agent can be formulated together into a single pharmaceutical composition or in separate compositions. In a preferred embodiment, the antigen of interest and the modulatory agent are administered simultaneously to the subject. Alternatively, in certain situations it may be desirable to administer the antigen first and then the modulatory agent or vice versa (for example, in the case of an antigen that naturally evokes a Th1 response, it may be beneficial to first administer the antigen alone to stimulate a Th1 response and then administer an inhibitory agent, alone or together with a boost of antigen, to shift the immune response to a Th2 response).

VII. Diagnostic Assays

In another aspect, the invention features a method of diagnosing a subject for a disorder associated with aberrant biological activity or SLIM (e.g., that would benefit from modulation of, STAT expression and/or activity, modulation of IFN-γ, modulation of Th1 cell differentiation.

In one embodiment, the invention comprises identifying the subject as one that would benefit from modulation of STAT activity, e.g., modulation of the IFN-γ production or Th1 cell differentiation. For example, in one embodiment, expression of a SLIM can be detected in cells of a subject suspected of having a disorder associated with aberrant biological activity of STAT. The expression of a SLIM in cells of said subject could then be compared to a control and a difference in expression of SLIM in cells of the subject as compared to the control could be used to diagnose the subject as one that would benefit from modulation of an SLIM activity.

The “change in expression” or “difference in expression” of SLIM in cells of the subject can be, for example, a change in the level of expression of SLIM in cells of the subject as compared to a previous sample taken from the subject or as compared to a control, which can be detected by assaying levels of, e.g., SLIM mRNA, for example, by isolating cells from the subject and determining the level of SLIM mRNA expression in the cells by standard methods known in the art, including Northern blot analysis, microarray analysis, reverse-transcriptase PCR analysis and in situ hybridizations. For example, a biological specimen can be obtained from the patient and assayed for, e.g., expression or activity of SLIM. For instance, a PCR assay could be used to measure the level of SLIM in a cell of the subject. A level of SLIM higher or lower than that seen in a control or higher or lower than that previously observed in the patient indicates that the patient would benefit from modulation of a signal transduction pathway involving SLIM. Alternatively, the level of expression of SLIM in cells of the subject can be detected by assaying levels of, e.g., SLIM, for example, by isolating cells from the subject and determining the level of SLIM protein expression by standard methods known in the art, including Western blot analysis, immunoprecipitations, enzyme linked immunosorbent assays (ELISAs) and immunofluorescence. Antibodies for use in such assays can be made using techniques known in the art and/or as described herein for making intracellular antibodies.

In another embodiment, a change in expression of SLIM in cells of the subject results from one or more mutations (i.e., alterations from wildtype), e.g., the SLIM gene and mRNA leading to one or more mutations (i.e., alterations from wildtype) in the amino acid sequence of the protein. In one embodiment, the mutation(s) leads to a form of the molecule with increased activity (e.g., partial or complete constitutive activity). In another embodiment, the mutation(s) leads to a form of the molecule with decreased activity (e.g., partial or complete inactivity). The mutation(s) may change the level of expression of the molecule for example, increasing or decreasing the level of expression of the molecule in a subject with a disorder. Alternatively, the mutation(s) may change the regulation of the protein, for example, by modulating the interaction of the mutant protein with one or more targets e.g., resulting in a form of SLIM that cannot interact with a SLIM binding partner. Mutations in the nucleotide sequence or amino acid sequences of proteins can be determined using standard techniques for analysis of DNA or protein sequences, for example for DNA or protein sequencing, RFLP analysis, and analysis of single nucleotide or amino acid polymorphisms. For example, in one embodiment, mutations can be detected using highly sensitive PCR approaches using specific primers flanking the nucleic acid sequence of interest. In one embodiment, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364). This method can include the steps of collecting a sample of cells from a patient, isolating nucleic acid (e.g., genomic, DNA) from the cells of the sample, contacting the nucleic acid sample with one or more primers which specifically amplify a sequence under conditions such that hybridization and amplification of the sequence (if present) occurs, and detecting the presence or absence of an amplification product, or detecting the size of the amplification product and comparing the length to a control sample.

In one embodiment, the complete nucleotide sequence for SLIM can be determined. Particular techniques have been developed for determining actual sequences in order to study polymorphism in human genes. See, for example, Proc. Natl. Acad. Sci. U.S.A. 85, 544-548 (1988) and Nature 330, 384-386 (1987); Maxim and Gilbert. 1977. PNAS 74:560; Sanger 1977. PNAS 74:5463. In addition, any of a variety of automated sequencing procedures can be utilized when performing diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Restriction fragment length polymorphism mappings (RFLPS) are based on changes at a restriction enzyme site. In one embodiment, polymorphisms from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Moreover, the use of sequence specific ribozymes (see, for example, U.S. Pat. No. 5,498,531) can be used to score for the presence of a specific ribozyme cleavage site.

Another technique for detecting specific polymorphisms in particular DNA segment involves hybridizing DNA segments which are being analyzed (target DNA) with a complimentary, labeled oligonucleotide probe. See Nucl. Acids Res. 9, 879-894 (1981). Since DNA duplexes containing even a single base pair mismatch exhibit high thermal instability, the differential melting temperature can be used to distinguish target DNAs that are perfectly complimentary to the probe from target DNAs that only differ by a single nucleotide. This method has been adapted to detect the presence or absence of a specific restriction site, U.S. Pat. No. 4,683,194. The method involves using an end-labeled oligonucleotide probe spanning a restriction site which is hybridized to a target DNA. The hybridized duplex of DNA is then incubated with the restriction enzyme appropriate for that site. Reformed restriction sites will be cleaved by digestion in the pair of duplexes between the probe and target by using the restriction endonuclease. The specific restriction site is present in the target DNA if shortened probe molecules are detected.

Other methods for detecting polymorphisms in nucleic acid sequences include methods in which protection from cleavage agents is used to detect mismatched bases in RNA/RNA or RNA/DNA heteroduplexes (Myers et al. (1985) Science 230: 1242). In general, the art technique of “mismatch cleavage” starts by providing heteroduplexes of formed by hybridizing (labeled) RNA or DNA containing the polymorphic sequence with potentially polymorphic RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In another embodiment, alterations in electrophoretic mobility can be used to identify polymorphisms. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control nucleic acids can be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of nucleic acid molecule comprising polymorphic sequences in polyacrylamide gels containing a gradient of denaturant is assayed using denaturing gradient gel electrophoresis (DGGE) (Myers et al. (1985) Nature 313:495). When DGGE is used as the method of analysis, DNA can be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting polymorphisms include, but are not limited to, selective oligonucleotide hybridization, selective amplification, or selective primer extension. For example, oligonucleotide primers may be prepared in which the polymorphic region is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different polymorphisms when the oligonucleotides are attached to the hybridizing membrane and hybridized with labeled target DNA.

Another process for studying differences in DNA structure is the primer extension process which consists of hybridizing a labeled oligonucleotide primer to a template RNA or DNA and then using a DNA polymerase and deoxynucleoside triphosphates to extend the primer to the 5′ end of the template. Resolution of the labeled primer extension product is then done by fractionating on the basis of size, e.g., by electrophoresis via a denaturing polyacrylamide gel. This process is often used to compare homologous DNA segments and to detect differences due to nucleotide insertion or deletion. Differences due to nucleotide substitution are not detected since size is the sole criterion used to characterize the primer extension product.

Another process exploits the fact that the incorporation of some nucleotide analogs into DNA causes an incremental shift of mobility when the DNA is subjected to a size fractionation process, such as electrophoresis. Nucleotide analogs can be used to identify changes since they can cause an electrophoretic mobility shift. See, U.S. Pat. No. 4,879,214.

Many other techniques for identifying and detecting polymorphisms are known to those skilled in the art, including those described in “DNA Markers: Protocols, Applications and Overview,” G. Caetano-Anolles and P. Gresshoff ed., (Wiley-VCH, New York) 1997, which is incorporated herein by reference as if fully set forth.

In addition, many approaches have also been used to specifically detect SNPs. Such techniques are known in the art and many are described e.g., in DNA Markers: Protocols, Applications, and Overviews. 1997. Caetano-Anolles and Gresshoff, Eds. Wiley-VCH, New York, pp199-211 and the references contained therein). For example, in one embodiment, a solid phase approach to detecting polymorphisms such as SNPs can be used. For example an oligonucleotide ligation assay (OLA) can be used. This assay is based on the ability of DNA ligase to distinguish single nucleotide differences at positions complementary to the termini of co-terminal probing oligonucleotides (see, e.g., Nickerson et al. 1990. Proc. Natl. Acad. Sci. USA 87:8923. A modification of this approach, termed coupled amplification and oligonucleotide ligation (CAL) analysis, has been used for multiplexed genetic typing (see, e.g., Eggerding 1995 PCR Methods Appl. 4:337); Eggerding et al. 1995 Hum. Mutat. 5:153).

In another embodiment, genetic bit analysis (GBA) can be used to detect a SNP (see, e.g., Nikiforov et al. 1994. Nucleic Acids Res. 22:4167; Nikiforov et al. 1994. PCR Methods Appl. 3:285; Nikiforov et al. 1995. Anal Biochem. 227:201). In another embodiment, microchip electrophoresis can be used for high-speed SNP detection (see e.g., Schmalzing et al. 2000. Nucleic Acids Research, 28). In another embodiment, matrix-assisted laser desorption/ionization time-of-flight mass (MALDI TOF) mass spectrometry can be used to detect SNPs (see, e.g., Stoerker et al. Nature Biotechnology 18:1213).

In another embodiment, a difference in a biological activity of SLIM between a subject and a control can be detected. For example, an activity of SLIM can be detected in cells of a subject suspected of having a disorder associated with aberrant biological activity of SLIM. The activity of SLIM in cells of the subject could then be compared to a control and a difference in activity of SLIM in cells of the subject as compared to the control could be used to diagnose the subject as one that would benefit from modulation of an SLIM activity. Activities of SLIM can be detected using methods described herein or known in the art.

In preferred embodiments, the diagnostic assay is conducted on a biological sample from the subject, such as a cell sample or a tissue section (for example, a freeze-dried or fresh frozen section of tissue removed from a subject). In another embodiment, the level of expression SLIM in cells of the subject can be detected in vivo, using an appropriate imaging method, such as using a radiolabeled antibody.

In one embodiment, the level of expression of SLIM in cells of the test subject may be elevated (i.e., increased) relative to the control not associated with the disorder or the subject may express a constitutively active (partially or completely) form of the molecule. This elevated expression level of, e.g., SLIM or expression of a constitutively active form of SLIM, can be used to diagnose a subject for a disorder associated with increased SLIM activity.

In another embodiment, the level of expression of SLIM in cells of the subject may be reduced (i.e., decreased) relative to the control not associated with the disorder or the subject may express an inactive (partially or completely) mutant form of SLIM. This reduced expression level of SLIM or expression of an inactive mutant form of SLIM can be used to diagnose a subject for a disorder, such as immunodeficiency disorders characterized by insufficient cytokine production.

In another embodiment, an assay diagnosing a subject as one that would benefit from modulation of SLIM expression, post-translational modification, and/or activity (or a molecule in a signal transduction pathway involving SLIM is performed prior to treatment of the subject.

The methods described herein may be performed, for example, by utilizing pre-packaged diagnostic kits comprising at least one probe/primer nucleic acid or other reagent (e.g., antibody), which may be conveniently used, e.g., in clinical settings to diagnose patients exhibiting symptoms or family history of a disease or illness involving SLIM.

VII. Kits of the Invention

Another aspect of the invention pertains to kits for carrying out the screening assays, modulatory methods or diagnostic assays of the invention. For example, a kit for carrying out a screening assay of the invention can include an indicator composition comprising a SLIM, means for measuring a readout (e.g., protein secretion) and instructions for using the kit to identify modulators of biological effects of SLIM. In another embodiment, a kit for carrying out a screening assay of the invention can include cells deficient in SLIM, means for measuring the readout and instructions for using the kit to identify modulators of a biological effect of SLIM.

In another embodiment, the invention provides a kit for carrying out a modulatory method of the invention. The kit can include, for example, a modulatory agent of the invention (e.g., SLIM inhibitory or stimulatory agent) in a suitable carrier and packaged in a suitable container with instructions for use of the modulator to modulate a biological effect of SLIM.

Another aspect of the invention pertains to a kit for diagnosing a disorder associated with a biological activity of SLIM in a subject. The kit can include a reagent for determining expression of SLIM (e.g., a nucleic acid probe for detecting SLIM mRNA or an antibody for detection of SLIM protein), a control to which the results of the subject are compared, and instructions for using the kit for diagnostic purposes.

IX. Administration of SLIM Modulating Agents

SLIM modulating agents of the invention are administered to subjects in a biologically compatible form suitable for pharmaceutical administration in vivo to either enhance or suppress immune responses (e.g., T cell mediated immune responses). By “biologically compatible form suitable for administration in vivo” is meant a form of the protein to be administered in which any toxic effects are outweighed by the therapeutic effects of the modulating agent. The term subject is intended to include living organisms in which an immune response can be elicited, e.g., mammals. Examples of subjects include humans, dogs, cats, mice, rats, and transgenic species thereof, including but not limited to the transgenic SLIM mouse described herein. Administration of an agent as described herein can be in any pharmacological form including a therapeutically active amount of an agent alone or in combination with a pharmaceutically acceptable carrier.

Administration of a therapeutically active amount of the therapeutic compositions of the present invention is defined as an amount effective, at dosages and for periods of time necessary to achieve the desired result. For example, a therapeutically active amount of a SLIM modulating agent may vary according to factors such as the disease state, age, sex, and weight of the individual, and the ability of peptide to elicit a desired response in the individual. Dosage regimen may be adjusted to provide the optimum therapeutic response. For example, several divided doses may be administered daily or the dose may be proportionally reduced as indicated by the exigencies of the therapeutic situation.

The therapeutic or pharmaceutical compositions of the present invention can be administered by any suitable route known in the art including for example intravenous, subcutaneous, intramuscular, transdermal, intrathecal or intracerebral or administration to cells in ex vivo treatment protocols. Administration can be either rapid as by injection or over a period of time as by slow infusion or administration of slow release formulation. For treating tissues in the central nervous system, administration can be by injection or infusion into the cerebrospinal fluid (CSF). When it is intended that a SLIM modulator be administered to cells in the central nervous system, administration can be with one or more agents capable of promoting penetration of SLIM polypeptide across the blood-brain barrier.

The SLIM modulator can also be linked or conjugated with agents that provide desirable pharmaceutical or pharmacodynamic properties. For example, SLIM can be coupled to any substance known in the art to promote penetration or transport across the blood-brain barrier such as an antibody to the transferrin receptor, and administered by intravenous injection. (See for example, Friden et al., 1993, Science 259: 373-377 which is incorporated by reference). Furthermore, SLIM can be stably linked to a polymer such as polyethylene glycol to obtain desirable properties of solubility, stability, half-life and other pharmaceutically advantageous properties. (See for example Davis et al., 1978, Enzyme Eng 4: 169-73; Burnham, 1994, Am J Hosp Pharm 51: 210-218, which are incorporated by reference).

Furthermore, the SLIM modulator can be in a composition which aids in delivery into the cytosol of a cell. For example, the agent may be conjugated with a carrier moiety such as a liposome that is capable of delivering the peptide into the cytosol of a cell. Such methods are well known in the art (for example see Anselem et al., 1993, Chem Phys Lipids 64: 219-237, which is incorporated by reference). Alternatively, the SLIM modulator can be modified to include specific transit peptides or fused to such transit peptides which are capable of delivering the SLIM modulator into a cell. In addition, the agent can be delivered directly into a cell by microinjection.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. SLIM can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

Dose administration can be repeated depending upon the pharmacokinetic parameters of the dosage formulation and the route of administration used. It is also provided that certain formulations containing the SLIM modulator are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, olyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method for the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In one embodiment of this invention, a SLIM modulator may be therapeutically administered by implanting into patients vectors or cells capable of producing a biologically-active form of SLIM or a precursor of SLIM, i.e. a molecule that can be readily converted to a biological-active form of SLIM by the body. In one approach cells that secrete SLIM may be encapsulated into semipermeable membranes for implantation into a patient. The cells can be cells that normally express SLIM or a precursor thereof or the cells can be transformed to express SLIM or a biologically active fragment thereof or a precursor thereof. It is preferred that the cell be of human origin and that the SLIM polypeptide be human SLIM when the patient is human. However, the formulations and methods herein can be used for veterinary as well as human applications and the term “patient” or “subject” as used herein is intended to include human and veterinary patients.

Monitoring the influence of agents (e.g. drugs or compounds) on the expression or activity of a SLIM protein can be applied not only in basic drug screening, but also in clinical trials. For example, the effectiveness of an agent determined by a screening assay as described herein to increase SLIM gene expression, protein levels, or upregulate SLIM activity, can be monitored in clinical trials of subjects exhibiting decreased SLIM gene expression, protein levels, or downregulated SLIM activity. Alternatively, the effectiveness of an agent determined by a screening assay to decrease SLIM gene expression, protein levels, or downregulate SLIM activity, can be monitored in clinical trials of subjects exhibiting increased SLIM gene expression, protein levels, or upregulated SLIM activity. In such clinical trials, the expression or activity of a SLIM gene, and preferably, other genes that have been implicated in a disorder can be used as a “read out” or markers of the phenotype of a particular cell.

For example, and not by way of limitation, genes, including SLIM, that are modulated in cells by treatment with an agent (e.g., compound, drug or small molecule) which modulates SLIM activity (e.g., identified in a screening assay as described herein) can be identified. Thus, to study the effect of agents on a SLIM associated disorder, for example, in a clinical trial, cells can be isolated and RNA prepared and analyzed for the levels of expression of SLIM and other genes implicated in the SLIM associated disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of SLIM or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a SLIM protein, mRNA, or genomic DNA in the pre-administration sample; (iii) obtaining one or more post-administration samples from the subject; (iv) detecting the level of expression or activity of the SLIM protein, mRNA, or genomic DNA in the post-administration samples; (v) comparing the level of expression or activity of the SLIM protein, mRNA, or genomic DNA in the pre-administration sample with the SLIM protein, mRNA, or genomic DNA in the post administration sample or samples; and (vi) altering the administration of the agent to the subject accordingly. For example, increased administration of the agent may be desirable to increase the expression or activity of SLIM to higher levels than detected, i.e., to increase the effectiveness of the agent. Alternatively, decreased administration of the agent may be desirable to decrease expression or activity of SLIM to lower levels than detected, i.e. to decrease the effectiveness of the agent. According to such an embodiment, SLIM expression or activity may be used as an indicator of the effectiveness of an agent, even in the absence of an observable phenotypic response.

In a preferred embodiment, the ability of a SLIM modulating agent to modulate IFN-γ production in a cell of a subject that would benefit from modulation of the expression and/or activity of SLIM can be measured by detecting an improvement in the condition of the patient after the administration of the agent. Such improvement can be readily measured by one of ordinary skill in the art using indicators appropriate for the specific condition of the patient. Monitoring the response of the patient by measuring changes in the condition of the patient is preferred in situations were the collection of biopsy materials would pose an increased risk and/or detriment to the patient.

Furthermore, in the treatment of disease conditions, compositions containing SLIM can be administered exogenously and it would likely be desirable to achieve certain target levels of SLIM polypeptide in sera, in any desired tissue compartment or in the affected tissue. It would, therefore, be advantageous to be able to monitor the levels of SLIM polypeptide in a patient or in a biological sample including a tissue biopsy sample obtained form a patient and, in some cases, also monitoring the levels of SLIM and, in some circumstances, also monitoring levels of STAT, or another SLIM-interacting polypeptide, or IFN-γ. Accordingly, the present invention also provides methods for detecting the presence of SLIM in a sample from a patient.

This invention is further illustrated by the following example, which Should not be construed as limiting. The contents of all references, patents and published patent applications cited throughout this application are hereby incorporated by reference. Additionally, all nucleotide and amino acid sequences deposited in public databases referred to herein are also hereby incorporated by reference.

### EXAMPLES

This invention is further illustrated by the following examples which should not be construed as limiting. The contents of all references, patents and published patent applications cited throughout this application, as well as the Figures and the Sequence Listing, are incorporated herein by reference.

Methods and Materials

Yeast Two-Hybrid Screening

A LexA-based yeast two-hybrid screening system (Clontech) was used to isolate proteins that can interact with Stat4. The bait plasmid was prepared by subcloning a cDNA fragment encoding the N-terminal 133 amino acids of Stat4 into the pEG202 yeast expression plasmid. This bait plasmid was co-transfected into the yeast strain EGY48 along with the pSH17 reporter plasmid which has 8 tandemly-repeated LexA operator sites and LacZ gene, followed by transformation with a cDNA library from mouse Th1 cell clone stimulated with anti-CD3 antibody for 5 hours. Transformed yeast were selected on dropout plates and surviving colonies were then replated on X-Gal plates. Colonies that turned blue in the presence of galactose but not glucose were picked and plasmids were retrieved from these yeast colonies and subjected to sequencing.

Preparation of Primary Cells from Mice

Single cell suspensions were obtained by mechanical disruption of spleens, while collagenase was used for the preparation of NK cells and dendritic cells. CD4+ T cells, CD8+ T cells, B cells and dendritic cells were purified using anti-CD4, anti-CD8, anti-B220 or anti-CD11c beads, respectively, together with Magnetic Cell Sorter (MACS, Miltenyi Biotech). For NK cells, DX-5+ cells were first purified from spleen cells, stained with anti-TCRβ and anti-DX-5 and sorted for TCRβ-DX-5+ cells. Peritoneal macrophages were harvested by washing the peritoneal cavity with PBS 4 days after i.p. injection of 2 ml 10% proteose peptone.

Northern Blot Analysis and RT-PCR

Total RNA was isolated using TRIZOL reagent (Gibco/BRL), and 10 μg of each sample was separated on denaturing formaldehyde agarose gel and transferred to GeneScreen membrane (NEN). Probes were radiolabeled by random priming using DECAprime II (Ambion). Full length SLIM cDNA was used as a probe and β-actin or HPRT cDNA was used as a control. For RT-PCR analysis, total RNA was reverse transcribed using random hexamer and Superscript II reverse transcriptase (Gibco/BRmL) and then subjected to PCR analysis.

Expression Vectors, Recombinant Protein and Antibodies

Flag-STAT4 was generated in pCDNA3 (Invitrogen). His- and-Myc-SLIM were generated in pCDNA-His (Invitrogen) and pCMV-Myc (Clontech), respectively. (2×) IRF-1 luciferase reporter plasmid was a gift from T. Hoey. Flag-p53 and MDM2 were gifts from Z Yuan. For recombinant SLIM, His-SLIM was expressed in E. coli and subsequently purified on a Nickel column. To generate SLIM antisera, a GST-SLIM fusion protein was expressed in E. coli and immunized into rabbits. Anti-Stat4 (C-20; Santa Cruz), Omni-probe (M−21; Santa Cruz), anti-c-Myc (9E10; Santa Cruz), anti-HSP90 (H-114; Santa Cruz) and anti-Flag (M2, Sigma) were from the indicated sources.

Cells, Transfections and Reporter Assays

293T cells were maintained in DMEM (GibcoBRL). U3A cells were a gift from T. Hoey and maintained in DMEM. 2D6 cells were a gift from T. Hoey and grown in RPMI (GibcoBRL) supplemented with 15% FCS and 10% T-stim (BD Bioscience) in the presence of 250 pg/ml of IL-12. All transient transfections were carried out using Effectene (Qiagen). To assess the effect of SLIM on steady-state levels of STAT4 protein, 293T cells were transfected with STAT4 (0.1 μg) along with increasing amounts of Myc-SLIM and treated with IFNα for 30 min. For p53 experiments, 293T cells were transfected with p53 (0.11 g) along with SLIM or MDM2 (1.2 μg). Luciferase assays were carried out per the manufacture's protocol (Promega). To generate stable transformants of 2D6 cells, cells were transfected with pCMVMyc-SLIM or empty vector along with pCDNA3 by electroporation and selected in the presence of G418 (1 mg/ml) for 14 days. The clones that express high levels of SLIM were identified by Northern and Western blotting.

Western Blot Analysis and Immunopreceipitation

To determine the intracellular localization of SLIM, cytoplasmic and nuclear extracts were prepared separately as follows. Cells were first lysed by hypotonic buffer (20 mM HEPES pH78.0, 10 mM KCl, 1 mM MgCl, 0.1% Triton X-100, 20% glycerol, 10 mM Na3VO4, 1 mM PMSF, 10 μg/ml aprotinin, 1 μg/ml pepstatin, 10 μg/ml leupeptin), and incubated on ice for 10 minutes. After centrifugation at 5000 rpm, 4° C. for 5 minutes, supernatants were collected as cytoplasmic extracts. Nuclear extracts were prepared by resuspension of the crude nuclei in hypertonic buffer (20 mM HEPES pH8.0, 1 mM EDTA, 20% glycerol, 0.1% Triton X-100, 400 mM Nacl) by vortex at 4° C. for 30 minutes. The supernatants were collected as nuclear extracts after centrifugation at 14000 rpm, 4° C. for 5 minutes. These samples were resolved on 10% SDS-PAGE (BioRad) and transferred to OPTITRAN nitrocellulose membrane (Schleicher & Schuell). Blots were probed with the indicated antibody and developed using ECL (enhanced chemiluminescence) system (Amersham Pharmacia Biotech). The accuracy of separation was confirmed using antibodies specific for cytoplasmic (anti-HSP90, SantaCruz) and nuclear (Oct-1, SantaCruz) proteins. For detecting the interaction of SLIM with Stat4, cells were stimulated with human IFNα (1000 U/ml; PBL laboratories) for 30 minutes. Nuclear extracts were prepared as described above and immunoprecipitated with mouse monoclonal anti-6×His antibody (9E 10; Santa Cruz). Immunoprecipitates were then resolved on an 8% SDS-PAGE and transferred to nitrocellulose membrane. Blots were probed with rabbit polyclonal anti-Stat4 antibody (C-20; Santa Cruz). For phosphorylation analysis, cells were treated with human IFNα (1000 U/ml) for 30 minutes, and whole cell extracts were prepared as follows. Cells were lysed in 50 mM Tris pH8.0, 0.5% NP-40, 5 mM EDTA, 50 mM NaCl, 50 mM NaF, 10 mM Na3VO4, 1 mM PMSF, 10 μg/ml aprotinin, 1 μg/ml pepstatin, 10 μg/ml leupeptin, and rotated for 1 hour at 4° C. Supernatants were collected after centrifugation at 14000 rpm, 4° C. for 5 minutes. Cell extracts were then immunoprecipitated with rabbit polyclonal anti-Stat4 antibody (C-20; Santa Cruz). Then, immunoprecipitates were subjected to Western blotting and detected with mouse monoclonal anti-phospotyrosine antibody (4G10; Upstate biotechnology) or rabbit polyclonal anti-phosphoserine-Stat3 antibody (SantaCruz).

Luciferase Assay

For evaluating STAT-mediated gene transactivation, a luciferase reporter construct containing two copies of a high-affinity STAT site upstream of the herpes simplex virus thymidine kinase basal promoter (−50-+10) in pGL2 was used. The high affinity STAT sites are derived from the IRF-1 promoter, GCCGTATTTCGGGGAAATCA (SEQ ID NO:37). U3A cells were co-transfected with reporter and Stat4 together with or without SLIM. After 24 hours, cells were stimulated with human IFNα (1000 U/ml) for 5 hours and subjected to luciferase assay.

Ubiquitination Assays

For in vitro autoubiquitination, recombinant SLIM was incubated for 3 h with biotin-ubiquitin, E1 (Boston Biochem) and UbcH8 in ubiquitination buffer (50 mM Tris pH8.0, 50 mM NaCl, 1 mM ATP and 1 mM DTT) and subjected to Western blot with avidin-HRP. For in vitro ubiquitination assay of STAT4, STAT4, which was immunoprecipitated from ConA-activated thymocytes, was incubated with biotin-ubiquitin, E1 and UbcH5a (Boston Biochem) in the absence or presence of recombinant SLIM, and then immunoblotted with anti-STAT4. For in vivo ubiquitination assay of STAT4, 293T cells were transfected with STAT4 and Myc-SLIM and treated with MG132 and IFNα. His-tagged proteins were purified as previously described 16 and subjected to immunoblot with anti-STAT4. For in vivo ubiquitination assay of p53, 293T cells were transfected with p53 along with Myc-SLIM or MDM2. Whole cell extracts were prepared with RIPA buffer containing 10 mM N-ethylmaleimide and subjected to immunoprecipitation and immunoblot with anti-Flag.

Generation of SLIM-Deficient Mice

An 8 kb fragment encompassing exon 2 of murine SLIM genomic DNA was subcloned into pBluescript II KS(+). A targeting vector was constructed by inserting the neomycin phophotransferase (Neo) gene into exon 2. To enrich for homologous recombinants, the Poly A signal was removed from the Neo gene in the targeting vector. Homologous regions 5′ and 3′ of Neo were 1.5 kb and 6.5 kb, respectively. Embryonic stem (ES) cells were electroporated with linealized targeting vector and cells were plated on feeder layers and cultured in the presence of G418. Resistant clones were picked and homologous recombinants screened by Southern blot analysis. Correctly targeted clones were injected into 3.5 day postcoital blastocysts to generate chimeric mice. Chimeric mice were mated with C57BL/6 females to generate heterozygous mice. Homozygous mutant mice were obtained by intercrossing of heterozygous mice. Two independent lines of SLIM-deficient mice having identical phenotypes were generated from independently targeted ES cell clones.

In Vitro Th1 Cell Differentiation

Lymphocytes were cultured in RPMI (Mediatech) supplemented with 10% fetal calf serum (Hyclone), penicillin-streptomycin, sodium pyruvate, HEPES, L-glutamine (all from Mediatech) and 5×10⁻⁵ M 2-mercaptoethanol (Sigma). For CD4+ T cells, cells were activated in vitro with plate-bound anti-CD3E (0.2 μg/ml; 145-2C11, Pharmingen) along with anti-CD28 (1 μg/ml, Pharmingen), IL-2 (50 U/ml) and IL-12 (5 ng/ml, R&D). After 5 days, cells were harvested, washed and restimulated with plate-bound anti-CD3ε (0.2 μg/ml) for 24 hours. The supernatants were then collected. For total spleen cells, cells were cultured with HKLM (1×106 cfu/ml) for 4 days. Cells were then harvested, washed and restimulated with plate-bound anti-CD3ε (0.2 μg/ml or 1 μg/ml) for 24 hours, and then supernatants were collected. Cytokine production was measured by ELISA (Pharmingen).

In Vivo TH1 Cell Differentiation with HKLM

Listeria monocytogenes (ATCC19111) was cultured in Brain Heart Infusion media for 18 hours and subsequently incubated at 60° C. for 4 hours to make heat-killed Listeria monocytogenes (HKLM). Mice were i.p. injected with HKLM (1×10⁹ cfu) at day 0 and day 5. At day 10, livers were fixed in Bouin's fixative solution, sectioned at 6 μm and stained with hematoxylin and eosin. Total spleen cells were prepared at the same day and stimulated in vitro with anti-CD3ε for 24 hours. IFNγ production in the supernatant was measured by ELISA.

### Example 1

### Identification and Characterization of SLIM cDNA and Amino Acid Sequence

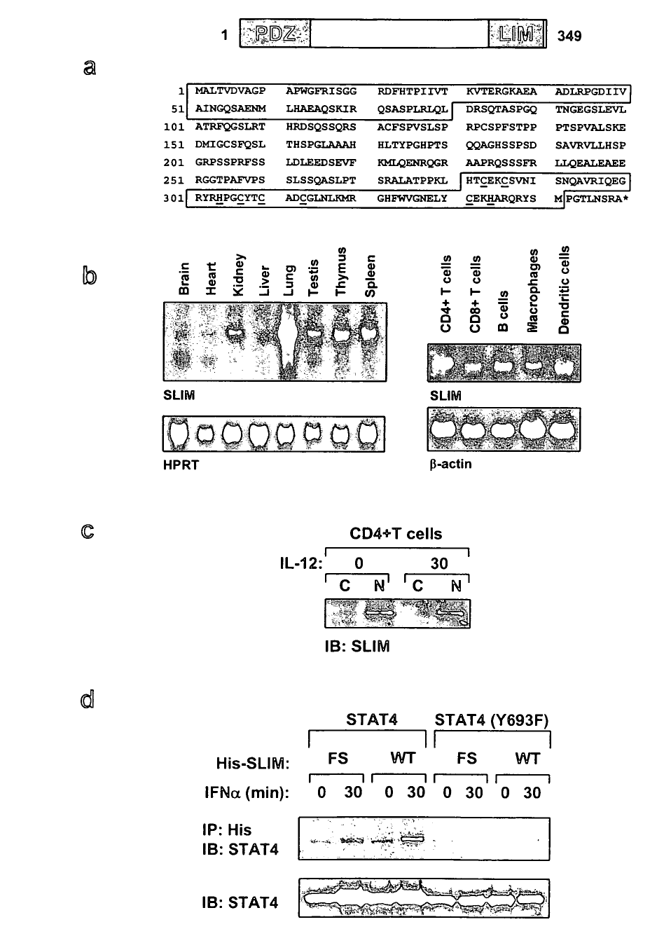

STAT proteins have several functional domains, such as a central DNA-binding domain, a conserved SH2 domain and a C-terminal transactivation domain (Hoey, T. and Grusby, M. J. (1999) Adv Immunol 71, 145-162.). To identify novel molecules that interact with STAT proteins, a composite yeast two-hybrid bait that contains the N-terminal 133 amino acid residues of Stat4 was generated. This region is highly conserved in STAT family members and has been shown to mediate the tetramerization of STAT dimers and other important protein-protein interactions that influence STAT function (Vinkemeier, U., et al. (1998) Science 279, 1048-1052). A cDNA library from a mouse Th1 cell clone stimulated for 5 hours with anti-CD3 was screened and 3 cDNA clones encoding proteins that could interact specifically with the N-terminal Stat4 bait, but not with other non-related baits, were isolated. One of these clones represents a novel protein that contains one PDZ domain at its N-terminus and one LIM domain at its C-terminus (FIG. 1A). The LIM domain is a specialized double-zinc finger motif that can interact with a number of different protein domains (Dawid, I. B., et al. (1998) Trends Genet 14, 156-162). In addition to the LIM domain, SLIM contains an N-terminal PDZ domain, which is also involved in protein-protein interactions (Fanning, A. S., and Anderson, J. M. (1996) Curr Biol 6, 1385-1-388), and thus SLIM is most similar in structure to RIL (Kiess, M., et al. (1995) Oncogene 10, 61-68.), ALP (Xia, H., et al. (1997) J Cell Biol 139,507-515.) and CLP-36 (Wang, H., et al. (1995) Gene 165, 267-271), which have one N-terminal PDZ domain and one C-terminal LIM domain, and Enigma (Wu, R. Y., and Gill, G. N. (1994) J Biol Chem 269, 25085-25090), ENE (Kuroda, S., et al. (1996) J Bio Chem 271, 31029-31032.) and ZASP/Cypher1 (Faulkner, G., et al. (1999) J Cell Biol 146, 465-475; Zhou, Q., et al. (1999) J Biol Chem 274, 19807-19813.1999), which have one N-terminal PDZ domain and three C-terminal LIM domains.). The cDNA encoding SLIM is 1506 bp and contains an open reading frame of 348 amino acids (FIG. 1A). Northern blot analysis of murine tissues revealed that SLIM mRNA expression is highest in lung, although it is also strongly expressed in spleen and thymus (FIG. 1B), and moderately in kidney and testis. Brain and heart express a smaller size SLIM mRNA which is possibly the result of alternative splicing.

Consistent with the fact that SLIM was isolated from a cDNA library of a Th1 cell clone, SLIM mRNA expression is also high in primary CD4+ T cells (FIG. 1B) although there is no difference in expression level between Th1 and Th2 cells. Other primary haematopoietic cells such as CD8+T cells, B cells, macrophages and dendritic cells also express SLIM mRNA (FIG. 1B). Western blot analysis using SLIM-specific polyclonal antisera revealed an approximately 38 kDa protein present in nuclear but not cytoplasmic extracts prepared from primary CD4+ T cells either before or after stimulation with IL-12 (FIG. 1C). To confirm the interaction of SLIM and STAT4 in mammalian cells, 293T cells were transiently transfected with expression plasmids encoding either an epitope-tagged wild type (WT) or a frame-shift (FS) mutant SLIM along with either a wild type or a tyrosine mutant (Y693F) STAT4. Transfectants were either left unstimulated or stimulated with IFNα for 30 min, and nuclear extracts were prepared and subjected to co-immunoprecipitation. As shown in FIG. 1D, STAT4 could be co-immunoprecipitated with SLIM only from nuclear extracts of cytokine stimulated cells and not from unstimulated cells. Moreover, STAT4 (Y693F), which is unable to be phosphorylated but which can translocate into the nucleus upon overexpression, could not be co-immunoprecipitated with SLIM. Taken together, these results suggest that SLIM is a nuclear protein that interacts with tyrosine phosphorylated STAT molecules that themselves have translocated into the nucleus following activation.

### Example 2

### Characterization of SLIM Function in STAT4-Mediated Signal Transduction

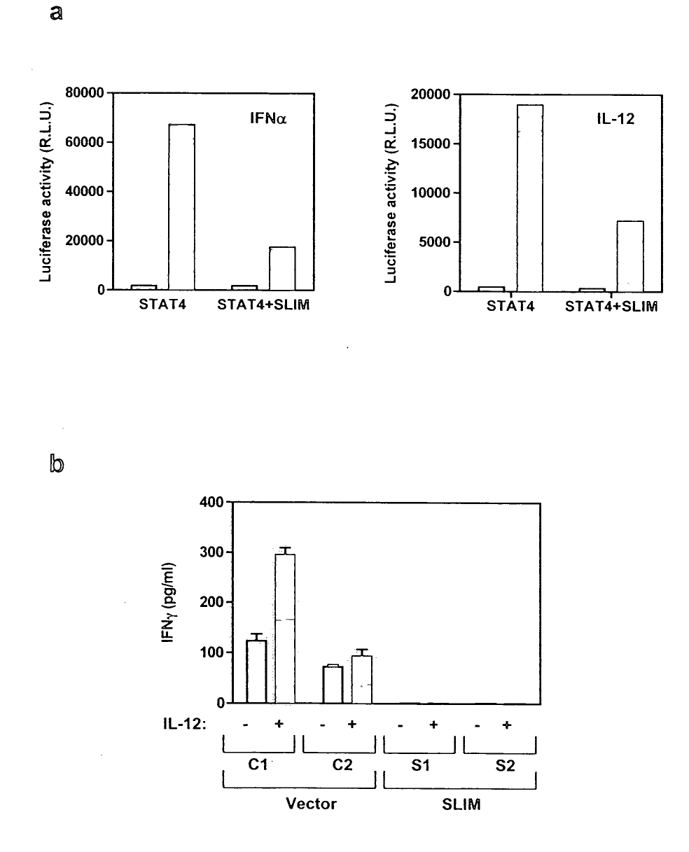

To examine the effect of SLIM on Stat4-mediated gene transactivation, U3A cells, which lack Stat1, were transiently transfected with a luciferase reporter constrict containing two copies of a high-affinity STAT site derived from the interferon regulatory factor-1 (IRF-1) promoter (Xu, X., et al. (1996) Science 273, 794-797) along with Stat4 and SLIM, and then stimulated with IFNα. Transfection transfection of reporter and Stat4 alone led to a robust increase in luciferase activity when the cells were stimulated with IFNα, while co-transfection of SLIM markedly impaired Stat4-mediated transactivaton of the reporter construct (FIG. 2A). Recently, the mechanism by which IFNα activates Stat4 has been shown to involve its interaction with the C-terminus of Stat2 at the IFNα receptor rather than through the generation of a Stat4 homodimer like that seen in response to IL-12 stimulation (Farrar, J. D., et al. (2000) Nat Immunol/1, 65-69). To examine if SLIM can inhibit Stat4-mediated transactivation in response to IL-12, U3A cells were stably transfected with human IL-12 receptor β1 and β2 chain expression constructs. These cells were then transiently transfected with the reporter construct along with Stat4 and SLIM and stimulated with human IL-12. Similar to that seen following stimulation with IFNα, transfection of reporter and Stat4 alone led to a robust increase in luciferase activity when the cells were stimulated with IL-12, while co-transfection of SLIM markedly impaired Stat4-mediated transactivaton of the reporter construct (FIG. 2A). Transfection of U3A cells with a reporter construct containing the SV-40 promoter and enhancer, but no STAT binding sites, and SLIM showed no effect on reporter activity demonstrating that SLIM is not a general inhibitor of transcription.

### Example 3

### SLIM Impairs the Tyrosine and Serine Phosphorylation of Stat4

To investigate the mechanism by which SLIM inhibits Stat4-mediated signal transduction, the effect of SLIM on the tyrosine and serine phosphorylation of Stat4 in response to cytokine stimulation was examined. 293T cells were transiently transfected with Stat4 and SLIM. Following stimulation with IFNα; total cell lysates were prepared, and the phosphorylation status of Stat4 was examined by Western blot analysis. The tyrosine phosphorylation of Stat4 following stimulation with IFNα was dramatically decreased when the cells were co-transfected with SLIM.

In addition to tyrosine 693, serine 721 of Stat4 is also phosphorylated upon IL-12 stimulation in an MKK6/p38 MAPK-dependent manner (Cho, S. S., et al. (1996) J Immunol 157, 4781-4789; Visconti, R., et al. (2000) Blood 96, 1844-1852). A recent study showed that serine phosphorylation is required for maximal Stat4 transcriptional activity but not for nuclear translocation and DNA binding activity and, in CD4+ T cells, Stat4 serine phosphorylation was found to be essential for IFNγ production but not for cell proliferation (Morinobu, A., et al. (2002) Proc Natl Acad Sci USA 99, 12281-12286.). To examine the effect of SLIM on serine phosphorylation of Stat4, the same cell lysates described above were subjected to Western blot analysis with a phosphoserine Stat3 antibody that cross-reacts with phosphoserine Stat4. Serine phosphorylation of Stat4 following stimulation with IFNαwas also dramatically decreased when the cells were co-transfected with SLIM. These data suggest that SLIM inhibits Stat4-mediated transactivation by impairing the tyrosine and serine phosphorylation of Stat4.

### Example 4

### SLIM Inhibits IL-12-Induced IFNγ Production in Th1 Cells

In order to determine if SLIM affects endogenous gene expression in response to Stat4 activation, 2D6 is a Th1 cell line that produces IFNγ in response to IL-12 stimulation in a Stat4-dependent manner (Ahn, H. J., et al. (1998) J Immunol 161, 5893-5900; Marno, S., et al. (1997) J Leukoc Biol 61, 346-352). 2D6 cells were stably transfected with a SLIM expression construct, or vector alone as control, and clones that exhibited high level SLIM expression by Northern and Western blot analysis were identified. IFNγ production in response to IL-12 stimulation was completely abolished in 2 independent 2D6 transfectants that overexpress SLIM (FIG. 2B) as compared to control cells. These transfectants produce levels of IFNγ comparable to control cells in response to stimulation with phorbol myristate acetate (PMA) plus ionomycin, demonstrating that these cells do not have a general defect in IFNγ expression. In addition, Jak2 phosphorylation in response to IL-12 stimulation was not affected in these cells, indicating that their IL-12 responsiveness was also not impaired. Furthermore, Western blot analysis of cytoplasmic and nuclear extracts prepared from 2D6 transfectants both before and after IL-12 stimulation revealed no defect in the nuclear translocation of Stat4 following cytokine stimulation. Nevertheless, the tyrosine phosphorylation of Stat4 in response to IL-12 stimulation was markedly impaired in 2D6 transfectants as compared to control cells. Taken together, these results demonstrate that SLIM does not affect the activation of Jak2 in response to IL-12 stimulation, nor the subsequent activation and nuclear translocation of Stat4. However, once in the nucleus, the phosphorylation of Stat4 is impaired and this leads to a marked inhibition in the expression of IFNγ, an endogenous Stat4 target gene.

### Example 5

### Identification of SLIM as a Ubiquitin E3 Ligase

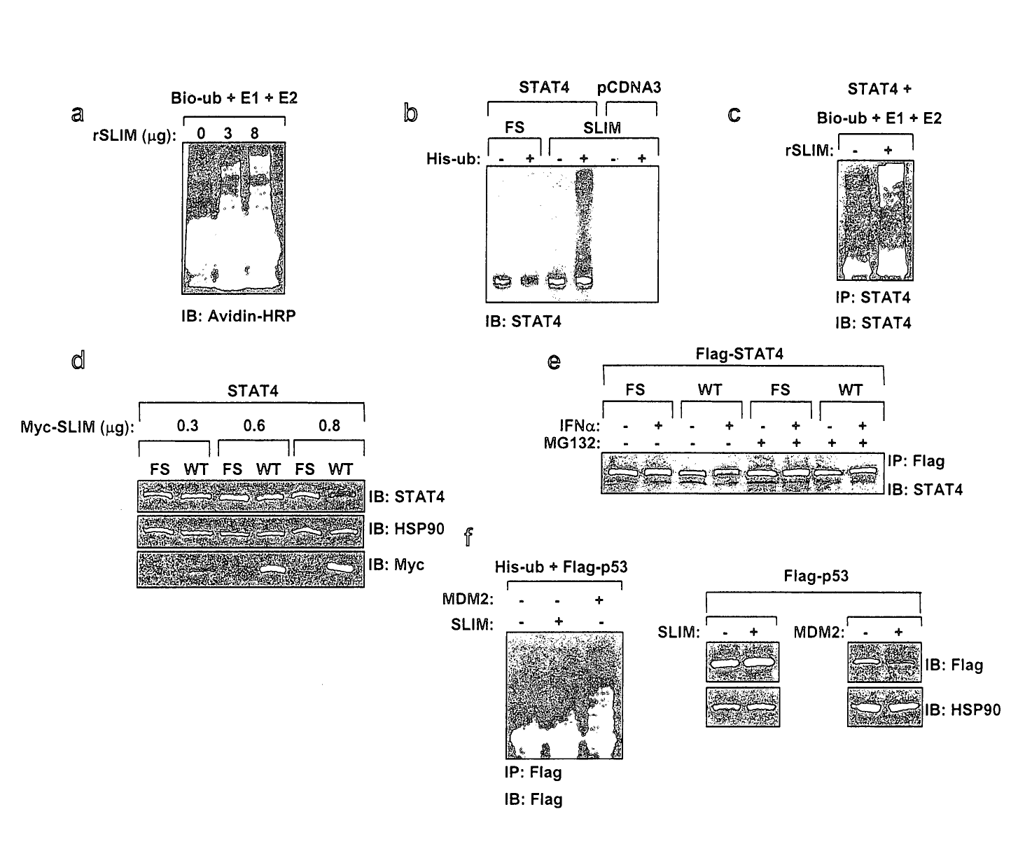

The LIM domain is thought to form a Zn finger structure not unlike that seen in related RING finger and PHD domains (Capili, A. D., et al. (2001) EMBO J 20, 165-177). Proteins containing these domains have been shown to possess ubiquitin E3 ligase activity and are involved in protein ubiquitination. To examine the mechanism by which SLIM inhibits STAT4-mediated signal transduction and the possibility that SLIM may function as an ubiquitin E3 ligase, purified recombinant epitope-tagged SLIM was mixed in vitro with E1, E2 and biotinylated ubiquitin and subjected to Western blot analysis. As shown in FIG. 3A, SLIM possess autoubiquitination activity as evidenced by the ladder of slower migrating ubiquitinated protein seen upon addition of SLIM. This ladder of ubiquitinated material was not seen if any one component of the in vitro ubiquitination assay was omitted, nor when a LIM domain-deletion mutant of SLIM was used.

To determine whether STAT proteins can be a target of SLIM-mediated ubiquitination, 293T cells were transfected with expression plasmids for SLIM, STAT4 and epitope-tagged ubiquitin. Whole cell extracts were prepared and ubiquitinated proteins were purified on Ni-NTA beads and subjected to Western blot analysis for STAT4. As shown in FIG. 3B, STAT4 was ubiquitinated in vivo only when the cells also expressed SLIM. In addition, purified recombinant SLIM could mediate the in vitro ubiquitination of purified STAT4 which was immunoprecipitated from ConA-activated thymocytes (FIG. 3C). Taken together, these data suggest that SLIM is an ubiquitin E3 ligase and that STAT proteins are a target of SLIM-mediated ubiquitination.

Ubiquitinated proteins are often degraded through a 26S proteosome-dependent pathway 13, and the potential for SLIM to affect the steady-state level of STAT4 protein expression was therefore assessed. Western blot analysis of 293T cells transfected with an expression plasmid for epitope-tagged STAT4 revealed a marked reduction in STAT4 protein expression upon co-transfection with an expression plasmid for SLIM (FIG. 3D). This decrease in STAT4 expression was not evident when the transfectants were treated with MG132, an inhibitor of the proteosome-dependent degradation pathway (FIG. 3E). In addition, there was no difference in STAT4 mRNA levels between cells co-transfected with wild type SLIM or a frame shift mutant of SLIM. Finally, this effect of SLIM was specific to STAT4 as SLIM could neither ubiquitinate nor induce the degradation of p53, while MDM2, an ubiquitin E3 ligase known to act on p53 (Li, M. et al. (2003) Science 302, 1972-1975), had these activities (FIG. 3F). Whether other proteins can be a target of SLIM-mediated ubiquitination remains to be determined. Taken together, however, these data suggest that SLIM can mediate the ubiquitination of STAT proteins and target them for proteosome-dependent degradation.

### Example 6

### Generation of SLIM −/− Mice

To further investigate the role of SLIM its vivo, SLIM −/− mice were generated by gene targeting in ES cells. Intercrossing of heterozygous animals yielded SLIM −/− mice at the expected Mendelian frequency. Northern and Western blot analysis of CD4+ T cells from SLIM −/− mice revealed that SLIM mRNA and protein, respectively, are not detected. SLIM −/− mice appeared healthy and fertile and no histological abnormalities were observed in any tissues of these mice, except for the liver of older mice. When fed a diet containing 15% fat, 100% of SLIM −/− mice older than 16-weeks displayed fatty liver, whereas only 20% of wild-type mice had the same phenotype. This may be due to an alteration of fat metabolism in SLIM −/− mice as we have observed SLIM expression in fat tissue.

Flow cytometric analysis of thymocytes from SLIM −/− mice revealed a normal ratio of T cells expressing CD4 and/or CD8, while SLIM −/− splenocytes contain normal frequencies of CD4+ T cells, CD8+ T cells, B cells, dendritic cells, macrophages and NK cells. Lymph nodes from SLIM −/− mice have comparable numbers of CD4+ T cells expressing CD62L, and CD69, CD25, CD44, are all normally induced by anti-CD3 plus anti-CD28 stimulation. In addition, the proliferative responses of SLIM −/− CD4+ T cells to anti-CD3 plus anti-CD28, IL-2 or IL-12 stimulation are comparable to wild-type cells.

### Example 7

### IFNγ Production is Enhanced in SLIM −/− Cells

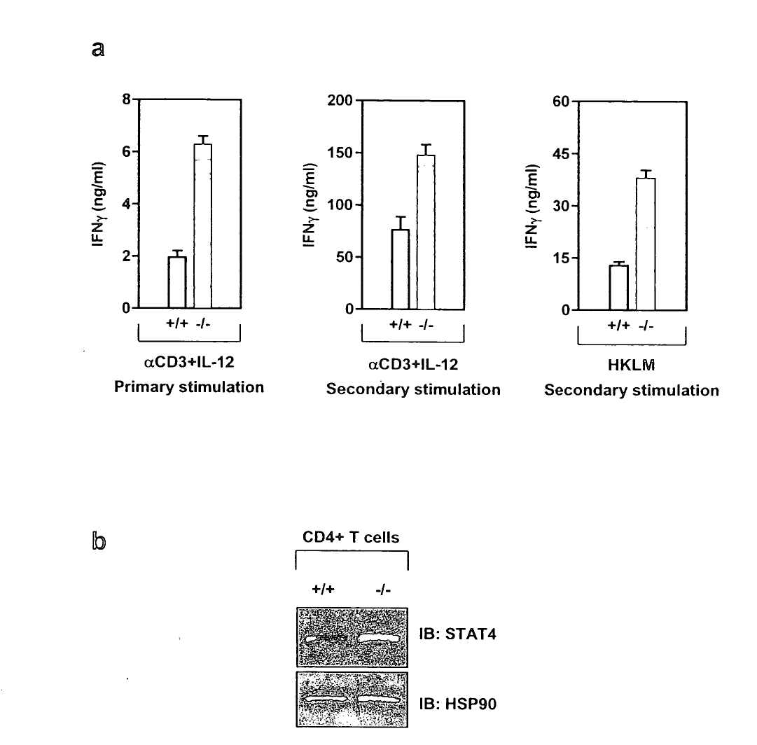

Given the importance of Stat4 signaling in the differentiation of Th1 cells and their subsequent production of IFNγ, it was of interest to examine this response in SLIM−/− T cells. CD4+ T cells were purified from lymph nodes of control and SLIM −/− mice and stimulated in vitro with anti-CD3 and IL-12. As shown in FIG. 4A, SLIM −/− Th1 cells produce 2-4 fold greater amounts of IFNγ as measured by ELISA following either primary or secondary stimulation as compared to control cells. Similar results were obtained when the cells were harvested after 5 days of culture and then restimulated with anti-CD3 and IL-12 for 24 hours, or when total spleen cells were stimulated in vitro with heat-killed Listeria monocytogenes (HKLM) in lieu of exogenous IL-12 to induce Th1 cell differentiation (Hsieh, C. S., et al. (1993) Science 260, 547-549) (FIG. 4B).

When examined by Western blot analysis, SLIM-deficient CD4+ T cells were found to express higher steady-state levels of STAT4 protein (FIG. 4A), consistent with the increased IFNγ production seen from these cells following activation. The response of SLIM−/− mice to in vivo administration of HKLM was also examined. Wild-type and SLIM −/− mice were injected i.p. with HKLM at day 0 and day 5, and the livers were then examined histologically at day 10. Whereas only a small number of mononuclear cells were found infiltrating the livers of wild-type mice, the livers of SLIM −/−mice had larger and greater numbers of focal accumulations of these cells. In addition, spleen cells from SLIM−/− mice produced significantly greater amounts of IFNγ when restimulated in vitro with anti-CD3 than did those from wild-type mice (data not shown). Taken together, these results suggest that IFNγ production by Th1 cells is enhanced both in vitro and in vivo in the absence of SLIM.

### Example 8

### Tyrosine and Serine Phosphorylation of Stat4 are Enhanced in SLIM −/− Cells

To examine the molecular basis for the enhanced Th1 cell differentiation and IFNγ production in SLIM −/− mice, splenic CD4+ T cells from wild-type and SLIM −/− mice were activated in vitro with anti-CD3 and IL-12 to induce expression of IL-12Rβ2. After 4 days, cells were harvested, washed and then cultured without IL-12 for an additional day. Tyrosine and serine phosphorylation of Stat4 in these cells were evaluated following restimulation with IL-12 for varying periods of time. Tyrosine phosphorylation of Stat4 in response to stimulation with IL-12 was enhanced in SLIM −/− CD4+ T cells compared to wild-type cells. Moreover, serine phosphorylation of Stat4 was barely detectable in wild-type CD4+ T cells, whereas it was markedly enhanced in SLIM −/'1 cells. These data suggest that the augmented IFNγ production by SLIM −/− cells is due to enhanced phosphorylation of Stat4 following IL-12 stimulation.

The results presented herein identify SLIM as a novel nuclear LIM-domain protein that functions as an E3 ubiquitin ligase and that regulates the steady-state levels of STAT4, and thus the IFNγ response in Vivo. SLIM is thus the first E3 ubiquitin ligase with specificity toward STAT proteins to be identified. The data presented herein demonstrate that SLIM can mediate the polyubiquitination of STAT4 and target it for proteosome-dependent degradation. Taken together, the data demonstrate that ubiquitination is an important mechanism for negatively regulating the STAT signaling pathway, and show that SLIM is an attractive target for manipulating cytokine responses in the treatment of autoimmune diseases.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule, comprising the coding sequence of the nucleotide sequence set forth in SEQ ID NO.:1, or a complement thereof.

2. The isolated nucleic acid sequence of claim 1, wherein the nucleic acid molecule is RNA.

3. An isolated nucleic acid molecule comprising the nucleotide sequence set forth in SEQ ID NO.:1, or a complement thereof.

4. An isolated nucleic acid molecule which has at least 95% identity to the nucleotide sequence set forth in SEQ ID NO.:1 over its full length and which encodes a polypeptide that binds to a STAT molecule.

5. An isolated nucleic acid molecule which has at least 95% identity to the nucleotide sequence set forth in SEQ ID NO.:1 over its full length and which encodes a polypeptide that modulates an activity selected from the group consisting of: STAT ubiquitination, STAT phosphorylation, IFN-γ production, STAT signaling, and Th1 cell differentiation.

6. An isolated nucleic acid molecule comprising a nucleotide sequence encoding a polypeptide comprising the amino acid sequence set forth in SEQ ID NO.:2.

7. An isolated nucleic acid molecule comprising the coding sequence of SEQ ID NO:1 and a nucleotide sequence encoding a non-SLIM polypeptide.

8. An isolated nucleic acid molecule which is complementary to the nucleic acid molecule of claim 1.

9. A vector comprising the nucleic acid molecule of claim 1.

10. The vector of claim 9, which is an expression vector.

11. A host cell containing the vector of claim 10.

12. A method for producing a polypeptide that binds to STAT, comprising culturing the host cell of claim 11 in a suitable medium until the polypeptide is produced.

13. An isolated polypeptide, comprising the amino acid sequence encoded by a nucleic acid molecule comprising the coding region of SEQ ID NO:1.

14. An isolated polypeptide comprising the amino acid sequence set forth in SEQ ID NO.:2.

15. An isolated protein consisting of the amino acid sequence of SEQ ID NO.:2.

16. An isolated polypeptide comprising an amino acid sequence which has at least 95% amino acid identity to the polypeptide set forth in SEQ ID NO:2 and binds to a STAT molecule.

17. A fusion protein comprising the amino acid sequence of SEQ ID NO:2 operatively linked to a non-SLIM polypeptide.

18. An antibody that specifically binds to a polypeptide encoded by the amino acid sequence set forth in SEQ ID NO.:2.

19. A transgenic mouse comprising in its genome an exogenous DNA molecule that functionally disrupts a nucleic acid molecule encoding a polypeptide comprising the amino acid sequence of SEQ ID NO.:2 in said mouse, wherein said mouse exhibits a phenotype characterized by increased IFN-γ production and increased phosphorylation of STAT4 relative to a wild-type mouse.

20. An isolated cell from the transgenic mouse of claim 19.

21. A method for identifying a compound that modulates the activity of a polypeptide comprising a consensus amino acid sequence shown in SEQ ID NO.:3, comprising
providing an indicator composition that comprises a nucleic acid molecule encoding the polypeptide operatively linked to a nucleotide sequence controlling its expression and a target molecule;
contacting the indicator composition with a library of test compounds;
determining the effect of the test compound on the expression and/or activity of the polypeptide in the indicator composition; and
selecting from the library of test compounds a compound of interest that modulates the expression and/or activity of the polypeptide;
to thereby identify a compound that modulates the activity of the polypeptide comprising the consensus amino acid sequences shown in SEQ ID NO.:3.

22. A method for identifying a compound which inhibits the E3 ubiquitin ligase activity of a polypeptide comprising a consensus amino acid sequence shown in SEQ ID NO.:3 comprising contacting in the presence of the compound, the polypeptide and a target molecule under conditions which allow ubiquitination of the target molecule by the polypeptide and detecting the target molecule in which the ability of the compound to inhibit the ubiquitination of the target molecule by the polypeptide is indicated by a decrease in ubiquitination of the target molecule as compared to the amount of ubiquitination of the target molecule in the absence of the compound.

23. A method for identifying a compound which inhibits the interaction of a polypeptide comprising a consensus amino acid sequence shown in SEQ ID NO.:3 with a STAT molecule comprising contacting in the presence of the compound, the polypeptide and the STAT molecule under conditions which allow binding of the STAT molecule to the polypeptide to form a complex; and detecting the formation of a complex of the polypeptide and the STAT molecule in which the ability of the compound to inhibit interaction between the polypeptide and the STAT molecule is indicated by a decrease in complex formation as compared to the amount of complex formed in the absence of the compound.

24. A method for identifying a compound that modulates the activity of a STAT molecule, comprising
providing an indicator composition that comprises a STAT molecule and a polypeptide comprising a consensus amino acid sequence shown in SEQ ID NO.:3 operatively linked to a nucleotide sequence controlling its expression;
contacting the indicator composition with a library of test compounds;
determining the effect of the test compound on the expression and/or activity of the polypeptide in the indicator composition; and
selecting from the library of test compounds a compound of interest that modulates the expression and/or activity of the polypeptide;
to thereby identify a compound that modulates the activity of a STAT molecule.

25. A method of modulating IFN-γ production by a cell comprising contacting the cell with an agent that modulates the expression and/or activity of a polypeptide comprising an amino acid sequence set forth in any one of SEQ ID NOs.:2, 5, 7, 9, 11, 13, 15, and 17, such that IFN-γ production by the cell is modulated

26. A method of treating or preventing a disorder that would benefit from treatment with an agent that modulates the activity of a STAT polypeptide, comprising administering to a subject with said disorder an agent that modulates the expression and/or activity of a polypeptide comprising an amino acid sequence set forth in any one of SEQ ID NOs.: 2, 5, 7, 9, 11, 13, 15, and 17, such that the disorder is treated or prevented.

27. A method of modulating protein folding, protein transport and/or protein secretion by a cell comprising contacting the cell with an agent that modulates the expression and/or activity of a polypeptide comprising an amino acid sequence set forth in any one of SEQ ID NOs.: 2, 5, 7, 9, 11, 13, 15, and 17, such that protein folding, protein transport and/or protein secretion is modulated

28. A method of modulating protein degradation by a cell comprising contacting the cell with an agent that modulates the expression and/or activity of a polypeptide comprising an amino acid sequence set forth in any one of SEQ ID NOs.: 2, 5, 7, 9, 11, 13, 15, and 17, such that protein degradation is modulated


SEQUENCE LISTING (37 sequences)

SEQ ID NO: 1 - DNA, 1506 bp, Mus musculus
  modified_base (1211): a, c, g, t, unknown or other
cttctccctttgctttggggtgtactgtggaagtgggccccacctcccagtcttctcttcctcaggcatggcgttgactgtggatgtggcaggaccagcaccttggggcttccgaattagcgggggcagagatttccacacacccatcattgtgaccaaggtcacagagcggggcaaggctgaagcagctgatctccggcctggcgacatcattgtggccatcaatggacagagtgcagagaacatgctacacgcggaggcccaaagcaagatccgacagagcgcctcacccctaagactgcagctggaccggtcccaaacagcctctcctgggcagaccaatggggagggctccttggaagtgctggcaaccagattccagggctccctgaggacacaccgtgacagccagtcttcccagaggtctgcctgcttcagcccagtctctctcagccccaggccttgcagccccttctccaccccaccccctaccagcccagttgccctttctaaagaggatatgattggctgtagtttccagagtctgacacactctccaggccttgctgctgctcaccacttgacctaccctggccaccccaccagccaacaggccggccacagcagcccaagcgactccgcagtgagggtgctgctccattccccaggacggccctccagccctaggttcagtttggatctggaggaagactcagaggtgttcaagatgctgcaggagaaccgccagggacgggccgccccaaggcagtccagctcttttcgactcttacaggaagccttggaggctgaggagagaggtggcacacctgcctttgtgcccagctcgctgagctcccaggcttccttgcccacctccagggccttggccactccacccaagctccacacctgtaagaaatgcagcgtcaacatctcgaaccaggcggtccgcatccaggaggggaggtaccgacaccctggctgctacacttgcgcagactgtgggctgaacctgaagatgcgcggccacttctgggtgggcaatgagttgtactgcgagaagcatgcccgccagcgctactctatgcctggaactctcaactctcgagcctgagcctcaaggtgctcggcctgtctgcactctcagactctgcagacatgattatactgagagcaagcagggaaggggtgatagcaggtgatagatgatnttacatgaactaaggttggggagtcccctttgtccttgctgggtgaggccaagggttgggactaatgtcaggttgctagtgctaaggacagttccactctctctggccttcctcctgcaggccaggttctgtattacggtctacagtggctgccatgtttgacacgaaagcgtatggggttgggcatggatagaagcatctagaagggaatggtgggcctgaggtaaatgatattcatggtgtgaagtttctaacatatgaactctatatacacgtggataaaattaaaaaaaaa

SEQ ID NO: 2 - protein, 348 aa, Mus musculus
MALTVDVAGPAPWGFRISGGRDFHTPIIVTKVTERGKAEAADLRPGDIIVAINGQSAENMLHAEAQSKIRQSASPLRLQLDRSQTASPGQTNGEGSLEVLATRFQGSLRTHRDSQSSQRSACFSPVSLSPRPCSPFSTPPPTSPVALSKEDMIGCSFQSLTHSPGLAAAHHLTYPGHPTSQQAGHSSPSDSAVRVLLHSPGRPSSPRFSLDLEEDSEVFKMLQENRQGRAAPRQSSSFRLLQEALEAEERGGTPAFVPSSLSSQASLPTSRALATPPKLHTCKKCSVNISNQAVRIQEGRYRHPGCYTCADCGLNLKMRGHFWVGNELYCEKHARQRYSMPGTLNSRA

SEQ ID NO: 3 - protein, 63 aa, Artificial Sequence
  Description of Artificial Sequence Synthetic LIM domain consensus sequence
CXXCXXXXXXXXXXXXXXXXXXXXXXXHXXCXXCXXCXXXXXXXXXXXXXXXXXXXXXCXXXX

SEQ ID NO: 4 - DNA, 1562 bp, Rattus norvegicus
cagacggctgcggtggaacggcggtggctcctcgtccttactccctctgttcttccctccctctttccctttcctggcgggcctggccgcccagcccaacaggcaagcggcagccaggcatggcgttgactgtgaatgtggtgggaccagcaccttggggcttccgcattagcggaggaagggatttccatacacccatcatcgtgacgaaggtcacagagaggggcaaggcggaggcagctgatctccggcctggcgacatcattgtggccatcaatggagagagtgccgagagcatgctacatgcggaggcccaaagcaagatccgacagagtgcctcacccctaagactgcagctggaccggtcccaaactgcctctcctgggcagatcaatggggagggctccttggacatgctggcaactagattccagggctccctgaggacacaccataacagccagtcttcccagagatctgcctgcttcagcccagcctctctcagtcccaggccagacagccctttctccactccaccccctaccagcccaattgccctttctggagagaatgtgattggctgtagtttccagagtctgacacactctccaggtcttgctgctactcaccacttgacctaccctggccagcccaccagccaacaggccggccacagcagcccaagcgactccacagtgagggtgctgcttcattccccaggacgaccctccagccccaggttaagcagtttggacctggaggaggactcagaggtgttcaagatgctgcaggagaaccgccagggacgggctgccccaaggcagtccagctcttttcggctcttacaggaagccttggaggctgaggaaagaggcggcacacctgcctttgtgcccagctcgctgagtcccaaggcttccttgcccacctccagggccttggccacgccacccaaactccacacctgtgagaagtgcagcgtcaacatctcgaaccaggcagtccgcatccaggaggggaggtaccgacaccctggctgctacacctgtgcagactgcgggctgaacctgaagatgcggggtcacttctgggtgggaaacgagttgtactgtgagaagcacgcccgccagcgctactcaatgcctggaactctcagctctcaagcctgagccaaaaggtgtctgcactctcagactctgcagacatgaccatactgagcaagcagggaaggggtgataatagcagttgatagaactaaggctgggagtcccctttgtccttgctgggtgaggccaagggttgggactagtggcagattgctagtgctgagaaaattccactctctctggcctttctcctgcaggccaggttctatactacagtctgaagtggccgccatatttgacaagtttgcatatagggttgggcacaggtagaagtatctagaagggaaaggtgggcctgaggttaatatattcatggtatgaagtttctaacatatgaactatatatacatgtggctaaaattaaaaaaaaaaaaaaaaaaaaaaaaaaaaaaaaaa

SEQ ID NO: 5 - protein, 349 aa, Rattus norvegicus
MALTVNVVGPAPWGFRISGGRDFHTPIIVTKVTERGKAEAADLRPGDIIVAINGESAESMLHAEAQSKIRQSASPLRLQLDRSQTASPGQINGEGSLDMLATRFQGSLRTHHNSQSSQRSACFSPASLSPRPDSPFSTPPPTSPIALSGENVIGCSFQSLTHSPGLAATHHLTYPGQPTSQQAGHSSPSDSTVRVLLHSPGRPSSPRLSSLDLEEDSEVFKMLQENRQGRAAPRQSSSFRLLQEALEAEERGGTPAFVPSSLSPKASLPTSRALATPPKLHTCEKCSVNISNQAVRIQEGRYRHPGCYTCADCGLNLKMRGHFWVGNELYCEKHARQRYSMPGTLSSQA

SEQ ID NO: 6 - DNA, 1562 bp, Rattus norvegicus; identical to SEQ ID NO: 4

SEQ ID NO: 7 - protein, 349 aa, Rattus norvegicus; identical to SEQ ID NO: 5

SEQ ID NO: 8 - DNA, 1857 bp, Homo sapiens
agagtcgggtagacggcagcgggagcggtggcgtctccccgccttccctccctcccgggcctgggcgcccagccggacaggtgagcggcagccaggtgagcgcgcccacctgcgcctctccgcgcggcccgccctccccggcgccgggctcctctccgcgcccctgtcggcgcggaaccctggcctcgtccgcggcccagctccctggagcctcgcatcagcgggggcgcccccgcgagctgcgctctccccggccggagcgctcctcctccagcccccagcccgcagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggacaaaataaaaaaaaaaaaa

SEQ ID NO: 9 - protein, 352 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPPKLHTCEKCSTSIANQAVRIQEGRYRHPGCYTCADCGLNLKMRGHFWVGDELYCEKHARQRYSAPATLSSRA

SEQ ID NO: 10 - DNA, 1772 bp, Homo sapiens
gccctccccggcgccgggctcctctccgcgcccctgtcggcgcggaaccctggcctcgtccgcggcccagctccctggagcctcgcgtcagcgggggcgcccccgcgagctgcgctctccccggccggagcgctcctcctccagcccccagcccgcagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggacaaaaaaaaaaaaaaaaaaaaaaaaaaaaaaaaaaaaaaaaaaaaaaaaaaaaaaaaaaaaaaa

SEQ ID NO: 11 - protein, 352 aa, Homo sapiens; identical to SEQ ID NO: 9

SEQ ID NO: 12 - DNA, 1869 bp, Homo sapiens
agagtcgggtagacggcagcgggagcggtggcgtctccccgccttccctccctcccgggcctgggcgcccagccggacaggggagcggcagccaggtgagcgcgcccacctgcgcctctccgcgcggcccgccctccccggcgccgggctcctctccgcgcccctgtcggcgcggaaccctggcctcgtccgcggcccagctccctggagcctcgcatcagcgggggcgcccccgcgagctgcgctctccccggccggagcgctcctcctccagcccccagcccgcagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcggccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgccagggtctaagtagggtcgaacacagaagtgggaaggagaggggtgggccaggggctaatggtgtcactgtgtaaagtttttgacatactagctctataaatatatgaatatggacaaaaaaaaaaaaaaaaaaaaaaaaaaaaaa

SEQ ID NO: 13 - protein, 352 aa, Homo sapiens; identical to SEQ ID NO: 9

SEQ ID NO: 14 - DNA, 1550 bp, Mus musculus
gagcaactgaagaggcaggaggcagctgcgtgggctctcaactccccagggcaagagttgccaggcatggcgttgactgtggatgtggcaggaccagcaccttggggcttccgaattagcgggggcagagatttccacacacccatcattgtgaccaaggtcacagagcggggcaaggctgaagcagctgatctccggcctggcgacatcattgtggccatcaatggacagagtgcagagaacatgctacacgcggaggcccaaagcaagatccgacagagcgcctcacccctaagactgcagctggaccggtcccaaacagcctctcctgggcagaccaatggggagggctccttggaagtgctggcaaccagattccagggctccctgaggacacaccgtgacagccagtcttcccagaggtctgcctgcttcagcccagtctctctcagccccaggccttgcagccccttctccaccccaccccctaccagcccagttgccctttctaaagaggatatgattggctgtagtttccagagtctgacacactctccaggccttgctgctgctcaccacttgacctaccctggccaccccaccagccaacaggccggccacagcagcccaagcgactccgcagtgagggtgctgctccattccccaggacggccctccagccctaggttcagcagtttggatctggaggaagactcagaggtgttcaagatgctgcaggagaaccgccagggacgggccgccccaaggcagtccagctcttttcgactcttacaggaagccttggaggctgaggagagaggtggcacacctgcctttgtgcccagctcgctgagctcccaggcttccttgcccacctccagggccttggccactccacccaagctccacacctgtgagaaatgcagcgtcaacatctcgaaccaggcggtccgcatccaggaggggaggtaccgacaccctggctgctacacttgcgcagactgtgggctgaacctgaagatgcgcggccacttctgggtgggcaatgagttgtactgcgagaagcatgcccgccagcgctactctatgcctggaactctcaactctcgagcctgagcctcaaggtgctcggcctgtctgcactctcagactctgcagacatgattatactgagagcaagcagggaaggggtgatagcaggtgatagatgatcttacatgaactaaggttggggagtcccctttgtccttgctgggtgaggccaagggttgggactaatgtcaggttgctagtgctaaggacagttccactctctctggccttcctcctgcaggccaggttctgtattacggtctacagtggctgccatgtttgacacgaaagcgtatggggttgggcatggatagaagcatctagaagggaatggtgggcctgaggtaaatgatattcatggtgtgaagtttctaacatatgaactctatatacacgtggataaaattaagtagtgtattttcaaaaaaaaaaaaaaaaaaaaaaaaaaaaaaaaaaaa

SEQ ID NO: 15 - protein, 349 aa, Mus musculus
MALTVDVAGPAPWGFRISGGRDFHTPIIVTKVTERGKAEAADLRPGDIIVAINGQSAENMLHAEAQSKIRQSASPLRLQLDRSQTASPGQTNGEGSLEVLATRFQGSLRTHRDSQSSQRSACFSPVSLSPRPCSPFSTPPPTSPVALSKEDMIGCSFQSLTHSPGLAAAHHLTYPGHPTSQQAGHSSPSDSAVRVLLHSPGRPSSPRFSSLDLEEDSEVFKMLQENRQGRAAPRQSSSFRLLQEALEAEERGGTPAFVPSSLSSQASLPTSRALATPPKLHTCEKCSVNISNQAVRIQEGRYRHPGCYTCADCGLNLKMRGHFWVGNELYCEKHARQRYSMPGTLNSRA

SEQ ID NO: 16 - DNA, 1550 bp, Mus musculus; identical to SEQ ID NO: 14

SEQ ID NO: 17 - protein, 349 aa, Mus musculus; identical to SEQ ID NO: 15

SEQ ID NO: 18 - protein, 52 aa, Mus musculus
CKKCSVNISNQAVRIQEGRYRHPGCYTCADCGLNLKMRGHFWVGNELYCEKH

SEQ ID NO: 19 - protein, 52 aa, Rattus norvegicus
CEKCSVNISNQAVRIQEGRYRHPGCYTCADCGLNLKMRGHFWVGNELYCEKH

SEQ ID NO: 20 - protein, 52 aa, Homo sapiens; identical to SEQ ID NO: 19

SEQ ID NO: 21 - protein, 77 aa, Mus musculus
TVDVAGPAPWGFRISGGRDFHTPIIVTKVTERGKAEAADLRPGDIIVAINGQSAENMLHAEAQSKIRQSASPLRLQL

SEQ ID NO: 22 - protein, 77 aa, Rattus norvegicus
TVNVVGPAPWGFRISGGRDFHTPIIVTKVTERGKAEAADLRPGDIIVAINGESAESMLHAEAQSKIRQSASPLRLQL

SEQ ID NO: 23 - protein, 77 aa, Homo sapiens
TVDVAGPAPWGFRITGGRDFHTPIMVTKVAERGKAKDADLRPGDIIVAINGESAEGMLHAEAQSKIRQSPSPLRLQL

SEQ ID NO: 24 - protein, 77 aa, Artificial Sequence
  Description of Artificial Sequence Synthetic PDZ domain consensus sequence
TVXVAGPAPWGFRIXGGRDFHTPIXVTKVXERGKAXXADLRPGDIIVAINGXSAEXMLHAEAQSKIRQSXSPLRLQL

SEQ ID NO: 25 - DNA, 21 bp, Artificial Sequence
  Description of Combined DNA/RNA Molecule Synthetic oligonucleotide
ggucacagagcggggcaagtt

SEQ ID NO: 26 - DNA, 21 bp, Artificial Sequence
  Description of Combined DNA/RNA Molecule Synthetic oligonucleotide
cuugccccgcucugugacctt

SEQ ID NO: 27 - DNA, 21 bp, Artificial Sequence
  Description of Combined DNA/RNA Molecule Synthetic oligonucleotide
gaugcgcggccacuucuggtt

SEQ ID NO: 28 - DNA, 21 bp, Artificial Sequence
  Description of Combined DNA/RNA Molecule Synthetic oligonucleotide
ccagaaguggccgcgcauctt

SEQ ID NO: 29 - DNA, 21 bp, Artificial Sequence
  Description of Combined DNA/RNA Molecule Synthetic oligonucleotide
uguggugggaccagcaccutt

SEQ ID NO: 30 - DNA, 21 bp, Artificial Sequence
  Description of Combined DNA/RNA Molecule Synthetic oligonucleotide
aggugcuggucccaccacatt

SEQ ID NO: 31 - DNA, 21 bp, Artificial Sequence
  Description of Combined DNA/RNA Molecule Synthetic oligonucleotide
ccugaagaugcggggucactt

SEQ ID NO: 32 - DNA, 21 bp, Artificial Sequence
  Description of Combined DNA/RNA Molecule Synthetic oligonucleotide
gugaccccgcaucuucaggtt

SEQ ID NO: 33 - DNA, 21 bp, Artificial Sequence
  Description of Combined DNA/RNA Molecule Synthetic oligonucleotide
gguggccgagcggggcaaatt

SEQ ID NO: 34 - DNA, 21 bp, Artificial Sequence
  Description of Combined DNA/RNA Molecule Synthetic oligonucleotide
uuugccccgcucggccacctt

SEQ ID NO: 35 - DNA, 21 bp, Artificial Sequence
  Description of Combined DNA/RNA Molecule Synthetic oligonucleotide
gcaugcccgccagcgcuactt

SEQ ID NO: 36 - DNA, 21 bp, Artificial Sequence
  Description of Combined DNA/RNA Molecule Synthetic oligonucleotide
guagcgcuggcgggcaugctt

SEQ ID NO: 37 - DNA, 20 bp, Artificial Sequence
  Description of Artificial Sequence Synthetic oligonucleotide
gccgtatttcggggaaatca